SEQUENCE LISTING (134 sequences)

SEQ ID NO: 1 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 2 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVLVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 3 - DNA, 31 bp, Homo sapiens
gcaacctgtctcacgccctctggctgttgcc

SEQ ID NO: 4 - DNA, 22 bp, Homo sapiens
agttagttctaaggcaaacctt

SEQ ID NO: 5 - DNA, 33 bp, Homo sapiens
ggccgaattcgcaacctgtctcacgccctctgg

SEQ ID NO: 6 - DNA, 36 bp, Homo sapiens
ggccgaattcggacagttcaaggtttgccttagaac

SEQ ID NO: 7 - protein, 490 aa, Gallus gallus
MHNLSAQPWQAKMANLTYDNVTLSNRSEVAIQPPTNYKTVELVFIATVTGSLSLVTVVGNILVMLSIKVNRQLQTVNNYFLFSLACADLIIGVFSMNLYTVYIIKGYWPLGAVVCDLWLALDYVVSNASVMNLLIISFDRYFCVTKPLTYPARRTTKMAGLMIAAAWILSFILWAPAILFWQFIVGKRTVHERECYIQFLSNPAVTFGTAIAAFYLPVVIMTVLYIHISLASRSRVRRHKPESRKERKGKSLSFFKAPPVKQNNNNSPKRAVEVKEEVRNGKVDDQPSAQTEATGQQEEKETSNESSTVSMTQTTKDKPTTEILPAGQGQSPAHPRVNPTSKWSKIKIVTKQTGTESVTAIEIVPAKAGASDHNSLSNSRPANVARKFASIARSQVRKKRQMAAREKKVTRTIFAILLAFILTWTPYNVMVLINTFCETCVPETVWSIGYWLCYVNSTINPACYALCNATFKKTFKHLLMCQYRNIGTAR

SEQ ID NO: 8 - protein, 488 aa, Caenorhabditis elegans
MCFAEKGEGAGEDVDHHSLFCPKKLVGNLKGFIRNQYHQHETIQILKGSALFLLVLWTIFANSLVFIVLYKNPRLQTVPNLLVGNLAFSDLALGLIVLPLSSVYAIAGEWVFPDALCEVFVSADILCSTASIWNLSIVGLDRYWAITSPVAYMSKRNKRTAGIMILSVWISSALISLAPLLGWKQTAQTPNLIYEKNNTVRQCTFLDLPSYTVYSATGSFFIPTLLMFFVYFKIYQAFAKHRARQIYRQKVIRKHIESTILHEISHVLPTSDEFAKEEEEEEDSESSGQVENGLGNGNDAIIEEDECEDEDSDEKRDDHTSMTTVTATVTGPTEAPYMKREAKISKSVPIEKESAIQKREAKPMRSVMAISYEKVKRHKNRKERIYRKSLQRKPKAISAAKERRGVKVLGIILGCFTVCWAPFFTMYVLVQFCKDCSPNAHIEMFITWLGYSNSAMNPIIYTVFNRDYQIALKRLFTSEKKPSSTSRV

SEQ ID NO: 9 - protein, 423 aa, Homo sapiens
MDLRATSSNDSNATSGYSDTAAVDWDEGENATGSGSLPDPELSYQIITSLFLGALILCSIFGNSCVVAAIALERSLQNVANYLIGSLAVTDLMVSVLVLPMAALYQVLNKWTLGQDICDLFIALDVLCCTSSILHLCAIALDRYWAITDPIDYVNKRTPRRAAVLISVTWLIGFSISIPPMLGWRSAEDRANPDACIISQDPGYTIYSTFGAFYIPLILMLVLYGRIFKAARFRIRKTVKKTEKAKASDMCLTLSPAVFHKRANGDAVSAEWKRGYKFKPSSPCANGAVRHGEEMESLEIIEVNSNSKTHLPLPNTPQSSSHENINEKTTGTRRKIALARERKTVKTLGIIMGTFIFCWLPFFIVALVLPFCAENCYMPEWLGAVINWLGYSNSLLNPIIYAYFNKDFQSAFKKILRCKFHRH

SEQ ID NO: 10 - protein, 421 aa, Mus musculus
MDMFSLGQGNNTTTSLEPFGTGGNDTGLSNVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRAPEDRSNPNECTISKDHGYTIYSTFGAFYIPLLLMLVLYGRIFRAARFRIRKTVKKVEKKGAGTSFGTSSAPPPKKSLNGQPGSGDCRRSAENRAVGTPCANGAVRQGEDDATLEVIEVHRVGNSKGDLPLPSESGATSYVPACLERKNERTAEAKRKMALARERKTVKTLGIIMGTFILCWLPFFIVALVLPFCESSCHMPELLGAIINWLGYSNSLLNPVIYAYFNKDFQNAFKKIIKCKFCR

SEQ ID NO: 11 - protein, 423 aa, Fugu rubripes; identical to SEQ ID NO: 9

SEQ ID NO: 12 - protein, 509 aa, Lymnaea stagnalis
MANFTFGDLALDVARMGGLASTPSGLRSTGLTTPGLSPTGLVTSDFNDSYGLTGQFINGSHSSRSRDNASANDTSATNMTDDRYWSLTVYSHEHLVLTSVILGLFVLCCIIGNCFVIAAVMLERSLHNVANYLILSLAVADLMVAVLVMPLSVVSEISKVWFLHSEVCDMWISVDVLCCTASILHLVAIAMDRYWAVTSIDYIRRRSARRILLMIMVVWIVALFISIPPLFGWRDPNNDPDKTGTCIISQDKGYTIFSTVGAFYLPMLVMMIIYIRIWLVARSRIRKDKFQMTKARLKTEETTLVASPKTEYSVVSDCNGCNSPDSTTEKKKRRAPFKSYGCSPRPERKKNRAKKLPENANGVNSNSSSSERLKQIQIETAEAFANGCAEEASIAMLERQCNNGKKISSNDTPYSRTREKLELKRERKAARTLAIITGAFLICWLPFFIIALIGPFVDPEGIPPFARSFVLWLGYFNSLLNPIIYTIFSPEFRSAFQKILFGKYRRGHR

SEQ ID NO: 13 - protein, 572 aa, Homo sapiens
MTFRDLLSVSFEGPRPDSSAGGSSAGGGGGSAGGAAPSEGPAVGGVPGGAGGGGGVVGAGSGEDNRSSAGEPGSAGAGGDVNGTAAVGGLVVSAQGVGVGVFLAAFILMAVAGNLLVILSVACNRHLQTVTNYFIVNLAVADLLLSATVLPFSATMEVLGFWAFGRAFCDVWAAVDVLCCTASILSLCTISVDRYVGVRHSLKYPAIMTERKAAAILALLWVVALVVSVGPLLGWKEPVPPDERFCGITEEAGYAVFSSVCSFYLPMAVIVVMYCRVYVVARSTTRSLEAGVKRERGKASEVVLRIHCRGAATGADGAHGMRSAKGHTFRSSLSVRLLKFSREKKAAKTLAIVVGVFVLCWFPFFFVLPLGSLFPQLKPSEGVFKVIFWLGYFNSCVNPLIYPCSSREFKRAFLRLLRCQCRRRRRRRPLWRVYGHHWRASTSGLRQDCAPSSGDAPPGAPLALTALPDPDPEPPGTPEMQAPVASRRKPPSAFREWRLLGPFRRPTTQLRAKVSSLSHKIRAGGAQRAEAACAQRSEVEAVSLGVPHEVAEGATCQAYELADYSNLRETDI

SEQ ID NO: 14 - protein, 562 aa, Mus musculus
MTFRDILSVTFEGPRASSSTGGSGAGGGAGTVGPEGPAVGGVPGATGGSAVVGTGSGEDNQSSTAEAGAAASGEVNGSAAVGGLVVSAQGVGVGVFLAAFILTAVAGNLLVILSVACNRHLQTVTNYFIVNLAVADLLLSAAVLPFSATMEVLGFWPFGRTFCDVWAAVDVLCCTASILSLCTISVDRYVGVRHSLKYPAIMTERKAAAILALLWAVALVVSVGPLLGWKEPVPPDERFCGITEEVGYAIFSSVCSFYLPMAVIVVMYCRVYVVARSTTRSLEAGIKREPGKASEVVLRIHCRGAATSAKGNPGTQSSKGHTLRSSLSVRLLKFSREKKAAKTLAIVVGVFVLCWFPFFFVLPLGSLFPQLKPSEGVFKVIFWLGYFNSCVNPLIYPCSSREFKRAFLRLLRCQCRRRRRRLWPSLRPPLASLDRRPALRLCPQPAHRTPRGSPSPHCTPRPGLRRHAGGAGFGLRPSKASLRLREWRLLGPLQRPTTQLRAKVSSLSHKFRSGGARRAETACALRSEVEAVSLNVPQDGAEAVICQAYEPGDLSNLRETDI

SEQ ID NO: 15 - protein, 499 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLRRKQSSKHALGYTLHPPSQAVEGQHKDMVRIPVGSRETFYRISKTDGVCEWKFFSSMPRGSARITVSKDQSSCTTARTKSRSVTRLECSGMILAHCNLRLPGSRDSPASASQAAGTTGDVPPGRRHQAQLIFVFLVETGFHHVGQDDLDLLTS

SEQ ID NO: 16 - protein, 429 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLRRKQSSKHALGYTLHPPSQAVEGQHKDMVRIPVGSRETFYRISKTDGVCEWKFFSSMPRGSARITVSKDQSSCTTARGHTPMT

SEQ ID NO: 17 - protein, 455 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARRGMDCRYFTKNCREHIKHVNFMMPPWRKGLEC

SEQ ID NO: 18 - protein, 466 aa, Rattus norvegicustus norvegicus
MVLLSENASEGSNCTHPPAPVNISKAILLGVILGGLIIFGVLGNILVILSVACHRHLHSVTHYYIVNLAVADLLLTSTVLPFSAIFEILGYWAFGRVFCNIWAAVDVLCCTASIMGLCIISIDRYIGVSYPLRYPTIVTQRRGVRALLCVWVLSLVISIGPLFGWRQPAPEDETICQINEEPGYVLFSALGSFYVPLAIILVMYCRVYVVAKRESRGLKSGLKTDKSDSEQVTLRIHRKNVPAEGGGVSSAKNKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLRRRQSSKHALGYTLHPPSQALEGQHRDMVRIPVGSGETFYKISKTDGVCEWKFFSSMPQGSARITVPKDQSACTTARVRSKSFLQVCCCVGSSAPRPEENHQVPTIKIHTISLGENGEEV

SEQ ID NO: 19 - protein, 466 aa, Mus musculus
MVLLSENASEGSNCTHPPAQVNISKAILLGVILGGLIIFGVLGNILVILSVACHRHLHSVTHYYIVNLAVADLLLTSTVLPFSAIFEILGYWAFGRVFCNIWAAVDVLCCTASIMGLCIISIDRYIGVSYPLRYPTIVTQRRGVRALLCVWALSLVISIGPLFGWRQQAPEDETICQINEEPGYVLFSALGSFYVPLTIILVMYCRVYVVAKRESRGLKSGLKTDKSDSEQVTLRIHRKNVPAEGSGVSSAKNKTHFSVRLLKFSREKKAAKTLGIVVGCFVLCWLPFFLVMPIGSFFPNFKPPETVFKIVFWLGYLNSCINPIIYPCSSQEFKKAFQNVLRIQCLRRRQSSKHALGYTLHPPSQAVEEQHRGMVRIPVGSGETFYKISKTDGVCEWKFFSSMPQGSARITMPKDQSACTTARVRSKSFLQVCCCVGSSTPRPEENHQVPTIKIHTISLGENGEEV

SEQ ID NO: 20 - protein, 466 aa, Bos taurus
MVFLSGNASDSSNCTHPPPPVNISKAILLGVILGGLILFGVLGNILVILSVACHRHLHSVTHYYIVNLAVADLLLTSTVLPFSAIFEILGYWAFGRVFCNVWAAVDVLCCTASIMGLCIISIDRYIGVSYPLRYPTIVTQKRGLMALLCVWALSLVISIGPLFGWRQPAPEDETICQINEEPGYVLFSALGSFYVPLTIILVMYCRVYVVAKRESRGLKSGLKTDKSDSEQVTLRIHRKNAQVGGSGVTSAKNKTHFSVRLLKFSREKKAAKTLGIVVGCFVLCWLPFFLVMPIGSFFPDFRPSETVFKIAFWLGYLNSCINPIIYPCSSQEFKKAFQNVLRIQCLRRKQSSKHTLGYTLHAPSHVLEGQHKDLVRIPVGSAETFYKISKTDGVCEWKIFSSLPRGSARMAVARDPSACTTARVRSKSFLQVCCCLGPSTPSHGENHQIPTIKIHTISLSENGEEV

SEQ ID NO: 21 - protein, 295 aa, Canis familiaris
MVFLSGNASDSSNCTHPPAPVNISKAILLGVILGGLIIFGVLGNILVILSVACHRHLHSVTHYYIVNLAVADLLLTSTVLPFSAIFEILGYWAFGRVFCNIWAAVDVLCCTASIMGLCIISIDRYIGVSYPLRYPTIVTQKRGLMALLCVWALSLVISIGPLFGWRQPAPEDETICQITEEPGYVLFSALGSFYVPLTIILVMYCRVYVVAKRESRGLKSGLKTDKSDSEQVTLRIHRKNAPVGGTGVSSAKNKTHFSVRLLKFSREKKAAKTLGIVVGCFVLCWLPFFLVMPIG

SEQ ID NO: 22 - protein, 466 aa, Oryctolagus cuniculus
MVFLSGNASDSSNCTHPPAPVNISKAILLGVILGGLILFGVLGNILVILSVACHRHLHSVTHYYIVNLAVADLLLTSTVLPFSAIFEILGYWAFGRVFCNIWAAVDVLCCTASIISLCVISIDRYIGVSYPLRYPTIVTQRRGLRALLCVWAFSLVISVGPLFGWRQPAPDDETICQINEEPGYVLFSALGSFYVPLTIILAMYCRVYVVAKRESRGLKSGLKTDKSDSEQVTLRIHRKNAPAGGSGVASAKNKTHFSVRLLKFSREKKAAKTLGIVVGCFVLCWLPFFLVMPIGSFFPDFKPPETVFKIVFWLGYLNSCINPIIYPCSSQEFKKAFQNVLKIQCLRRKQSSKHALGYTLHAPSQALEGQHKDMVRIPVGSGETFYKISKTDGVCEWKFFSSMPRGSARITVPKDQSACTTARVRSKSFLQVCCCVGPSTPNPGENHQVPTIKIHTISLSENGEEV

SEQ ID NO: 23 - protein, 466 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLCRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEV

SEQ ID NO: 24 - protein, 470 aa, Oryzias latipes
MTPSSVTLNCSNCSHVLAPELNTVKAVVLGMVLGIFILFGVIGNILVILSVVCHRHLQTVTYYFIVNLAVADLLLSSTVLPFSAIFEILDRWVFGRVFCNIWAAVDVLCCTASIMSLCVISVDRYIGVSYPLRYPAIMTKRRALLAVMLLWVLSVIISIGPLFGWKEPAPEDETVCKITEEPGYAIFSAVGSFYLPLAIILAMYCRVYVVAQKESRGLKEGQKIEKSDSEQVILRMHRGNTTVSEDEALRSRTHFALRLLKFSREKKAAKTLGIVVGCFVLCWLPFFLVLPIGSIFPAYRPSDTVFKITFWLGYFNSCINPIIYLCSNQEFKKAFQSLLGVHCLRMTPRAHHHHLSVGQSQTQGHSLTISLDSKGAPCRLSPSSSVALSRTPSSRDSREWRVFSGGPINSGPGPTEAGRAKVAKLCNKSLHRTCCCILRARTPTQDPAPLGDLPTIKIHQLSLSEKGESV

SEQ ID NO: 25 - protein, 391 aa, Branchiostoma lanceolatum
MSANTTVSPTETTANLTANSTEASVGSCFAPNPYSAGVQAVLGLITVILILLTVIGNVLVILAVTCHRKMRTVTNFFIVSLACADLSVGITVLPFAATNDILGYWPFGGYCDVWVSFDVLNSTASILNLVVIAFDRFLAITAPFTYHTRMTERTAGILIATVWGISLVVSFLPIQAGWYRDNQSEEALAIYSDPCLCIFTASTAYTIVSSLISFYIPLLIMLVFYGIIFKAARDQARKINALEGRLEQENNRGKKISLAKEKKAAKTLGIIMGVFILCWLPFFVVNIVNPFCDRCVQPAVFIALTWLGWINSCFNPIIYAFNKEFRKVFVKMICCHKCRGVTVGPNHADLNYDPVAMRLKKRGENANGTVNGDANGKANGNIEAGEGTSSS

SEQ ID NO: 26 - protein, 36 aa, Homo sapiens
MTSTCTNSTRESNSSHTCMPLSKMPISLAHGIIRST

SEQ ID NO: 27 - protein, 13 aa, Homo sapiens
QRKPQLLQVTNRF

SEQ ID NO: 28 - protein, 5 aa, Homo sapiens
WPLNS

SEQ ID NO: 29 - protein, 20 aa, Homo sapiens
DRYLSIIHPLSYPSKMTQRR

SEQ ID NO: 30 - protein, 23 aa, Homo sapiens
GQAAFDERNALCSMIWGASPSYT

SEQ ID NO: 31 - protein, 182 aa, Homo sapiens
CAARRQHALLYNVKRHSLEVRVKDCVENEDEEGAEKKEEFQDESEFRRQHEGEVKAKEGRMEAKDGSLKAKEGSTGTSESSVEAGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAK

SEQ ID NO: 32 - protein, 15 aa, Homo sapiens
AVLAVWVDVETQVPQ

SEQ ID NO: 33 - protein, 55 aa, Homo sapiens
YGYMHKTIKKEIQDMLKKFFCKEKPPKEDSHPDLPGTEGGTEGKIVPSYDSATFP

SEQ ID NO: 34 - DNA, 20 bp, Homo sapiens
gcagagcactcctccactct

SEQ ID NO: 35 - DNA, 20 bp, Homo sapiens
agcaggcaatcatgacaatc

SEQ ID NO: 36 - DNA, 20 bp, Homo sapiens
gttagcctcacccacctgtt

SEQ ID NO: 37 - DNA, 20 bp, Homo sapiens
cacaatccaggtgccataga

SEQ ID NO: 38 - DNA, 42 bp, Homo sapiens
gtccccaagcttgcaccatgacgtccacctgcaccaacagca

SEQ ID NO: 39 - DNA, 62 bp, Homo sapiens
cgggatcctacttgtcgtcgtcgtccttgtagtccataggaaaagtagcagaatcgtaggaa

SEQ ID NO: 40 - protein, 407 aa, Homo sapiens
MSLNSSLSCRKELSNLTEEEGGEGGVIITQFIAIIVITIFVCLGNLVIVVTLYKKSYLLTLSNKFVFSLTLSNFLLSVLVLPFVVTSSIRREWIFGVVWCNFSALLYLLISSASMLTLGVIAIDRYYAVLYPMVYPMKITGNRAVMALVYIWLHSLIGCLPPLFGWSSVEFDEFKWMCVAAWHREPGYTAFWQIWCALFPFLVMLVCYGFIFRVARVKARKVHCGTVVIVEEDAQRTGRKNSSTSTSSSGSRRNAFQGVVYSANQCKALITILVVLGAFMVTWGPYMVVIASEALWGKSSVSPSLETWATWLSFASAVCHPLIYGLWNKTVRKELLGMCFGDRYYREPFVQRQRTSRLFSISNRITDLGLSPHLTALMAGGQPLGHSSSTGDTGFSCSQDSGNLRAL

SEQ ID NO: 41 - protein, 448 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAKKVIFIIIFSYVLSLGPYCFLAVEDSHPDLPGTEGGTEGKIVPSYDSATFP

SEQ ID NO: 42 - protein, 448 aa, Homo sapiens; identical to SEQ ID NO: 41

SEQ ID NO: 43 - protein, 448 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVLVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDGMEFGEDDINFSEDDVEAVNIPESLPPSRRNSNSNPPLPRCYQCKAAKVIFIIIFSYVLSLGPYCFLAVEDSHPDLPGTEGGTEGKIVPSYDSATFP

SEQ ID NO: 44 - DNA, 1659 bp, Homo sapiens
gcctgcaacctgtcycacgccctctggctgttgccatgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatggcatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttgaagttagttctaaggcaaaccttgaaaatcagtccttcagccacagctatttagagctttaaaactaccaggttcaatcactggttatgctttctgtg

SEQ ID NO: 45 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 46 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctaagaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 47 - DNA, 1580 bp, Homo sapiens
gcaacctgtctcacgccctctggctgttgccatgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttgaagttagttctaaggcaaacctt

SEQ ID NO: 48 - DNA, 1527 bp, Homo sapiens
  misc_feature (370)..(370): n is a, c, g, or t
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcntggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgncatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctnnnaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 49 - protein, 508 aa, Homo sapiens
  misc_feature (124)..(124): Xaa can be any naturally occurring amino acid
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVXVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDXMEFGEDDINFSEDDVEAVNIPESLPPSRRNSNSNPPLPRCYQCKAXKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 50 - DNA, 21 bp, Homo sapiens
caccattgtcttggtgtcagt

SEQ ID NO: 51 - DNA, 21 bp, Homo sapiens
caccattgtcgtggtgtcagt

SEQ ID NO: 52 - DNA, 21 bp, Homo sapiens
ggtgaagatgacatggagttt

SEQ ID NO: 53 - DNA, 21 bp, Homo sapiens
ggtgaagatggcatggagttt

SEQ ID NO: 54 - DNA, 21 bp, Homo sapiens
gtgcaaagctgctaaagtgat

SEQ ID NO: 55 - DNA, 21 bp, Homo sapiens
gtgcaaagctactaaagtgat

SEQ ID NO: 56 - DNA, 21 bp, Homo sapiens
tgcaaagctgctaaagtgatc

SEQ ID NO: 57 - DNA, 21 bp, Homo sapiens
tgcaaagctgataaagtgatc

SEQ ID NO: 58 - DNA, 21 bp, Homo sapiens
gcaaagctgctaaagtgatct

SEQ ID NO: 59 - DNA, 21 bp, Homo sapiens
gcaaagctgcgaaagtgatct

SEQ ID NO: 60 - DNA, 17 bp, Homo sapiens
agccgagccacatcgct

SEQ ID NO: 61 - DNA, 19 bp, Homo sapiens
gtgaccaggcgcccaatac

SEQ ID NO: 62 - DNA, 28 bp, Homo sapiens
caaatccgttgactccgaccttcacctt

SEQ ID NO: 63 - DNA, 99 bp, Artificial Sequence
  Synthesized Oligonucleotide.
cgaagcgtaagggcccagccggccnnbnnbnnbnnbnnbnnbnnbnnbnnbnnbnnbnnbnnbnnbnnbnnbnnbnnbnnbnnbccgggtccgggcggc

SEQ ID NO: 64 - DNA, 95 bp, Artificial Sequence
  Synthesized Oligonucleotide.
aaaaggaaaaaagcggccgcvnnvnnvnnvnnvnnvnnvnnvnnvnnvnnvnnvnnvnnvnnvnnvnnvnnvnnvnnvnngccgcccggacccgg

SEQ ID NO: 65 - protein, 5 aa, Homo sapiens
PGPGG

SEQ ID NO: 66 - protein, 15 aa, Homo sapiens
GDFWYEACESSCAFW

SEQ ID NO: 67 - protein, 15 aa, Homo sapiens
LEWGSDVFYDVYDCC

SEQ ID NO: 68 - protein, 15 aa, Homo sapiens
CLRSGTGCAFQLYRF

SEQ ID NO: 69 - protein, 15 aa, Homo sapiens
NNFPCLRSGRNCDAG

SEQ ID NO: 70 - protein, 15 aa, Homo sapiens
RIVPNGYFNVHGRSL

SEQ ID NO: 71 - protein, 14 aa, Homo sapiens
RIDSCAKYFLRSCD

SEQ ID NO: 72 - DNA, 39 bp, Homo sapiens
gcagcagcggccgcaccgtgctggttatcttcctcgccg

SEQ ID NO: 73 - DNA, 35 bp, Homo sapiens
gcagcagtcgacaggaaaagtagcagaatcgtagg

SEQ ID NO: 74 - DNA, 38 bp, Homo sapiens
gcagcagcggccgcatgacgtccacctgcaccaacagc

SEQ ID NO: 75 - DNA, 37 bp, Homo sapiens
gcagcagtcgacatagacataggggtggatgcagcac

SEQ ID NO: 76 - protein, 13 aa, Homo sapiens
STCTNSTRESNSS

SEQ ID NO: 77 - protein, 13 aa, Homo sapiens
QLLQVTNRFIFNL

SEQ ID NO: 78 - protein, 13 aa, Homo sapiens
YPSKMTQRRGYLL

SEQ ID NO: 79 - protein, 13 aa, Homo sapiens
EAKDGSLKAKEGS

SEQ ID NO: 80 - protein, 13 aa, Homo sapiens
EGKEGSTKVEENS

SEQ ID NO: 81 - protein, 13 aa, Homo sapiens
KVEENSMKADKGR

SEQ ID NO: 82 - protein, 13 aa, Homo sapiens
ESLPPSRRNSNSN

SEQ ID NO: 83 - protein, 13 aa, Homo sapiens
GYMHKTIKKEIQD

SEQ ID NO: 84 - protein, 14 aa, Homo sapiens
STCTNSTRESNSSH

SEQ ID NO: 85 - protein, 14 aa, Homo sapiens
TGTSESSVEARGSE

SEQ ID NO: 86 - protein, 14 aa, Homo sapiens
GKEGSTKVEENSMK

SEQ ID NO: 87 - protein, 14 aa, Homo sapiens
DDINFSEDDVEAVN

SEQ ID NO: 88 - protein, 14 aa, Homo sapiens
PPKEDSHPDLPGTE

SEQ ID NO: 89 - protein, 14 aa, Homo sapiens
LLYNVKRHSLEVRV

SEQ ID NO: 90 - protein, 14 aa, Homo sapiens
SLPPSRRNSNSNPP

SEQ ID NO: 91 - protein, 14 aa, Homo sapiens
TSTCTNSTRESNSS

SEQ ID NO: 92 - protein, 14 aa, Homo sapiens
STRESNSSHTCMPL

SEQ ID NO: 93 - protein, 14 aa, Homo sapiens
GEDDINFSEDDVEA

SEQ ID NO: 94 - protein, 16 aa, Homo sapiens
ISLAHGIIRSTVLVIF

SEQ ID NO: 95 - protein, 16 aa, Homo sapiens
CSMIWGASPSYTILSV

SEQ ID NO: 96 - protein, 16 aa, Homo sapiens
MEAKDGSLKAKEGSTG

SEQ ID NO: 97 - protein, 16 aa, Homo sapiens
LKAKEGSTGTSESSVE

SEQ ID NO: 98 - protein, 16 aa, Homo sapiens
KEGSTGTSESSVEARG

SEQ ID NO: 99 - protein, 16 aa, Homo sapiens
TVASDGSMEGKEGSTK

SEQ ID NO: 100 - protein, 16 aa, Homo sapiens
HPDLPGTEGGTEGKIV

SEQ ID NO: 101 - protein, 16 aa, Homo sapiens
LPGTEGGTEGKIVPSY

SEQ ID NO: 102 - protein, 21 aa, Homo sapiens
SVVSFIVIPLIVMIACYSVVF

SEQ ID NO: 103 - protein, 372 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKSRNDSKSSYQFEDAIGACVIIL

SEQ ID NO: 104 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 105 - DNA, 18 bp, Homo sapiens
ttcctcgccgcctctttc

SEQ ID NO: 106 - DNA, 15 bp, Homo sapiens
cggcttgcgctgcaa

SEQ ID NO: 107 - DNA, 25 bp, Homo sapiens
actagcgccagcactatgttgccga

SEQ ID NO: 108 - DNA, 24 bp, Homo sapiens
cacagccatcaacttcttcctcta

SEQ ID NO: 109 - DNA, 15 bp, Homo sapiens
ggcggctgccatggt

SEQ ID NO: 110 - DNA, 23 bp, Homo sapiens
tgcttcatcagcaagcggttccg

SEQ ID NO: 111 - DNA, 23 bp, Homo sapiens
aagtggatgtcaactacgcgttt

SEQ ID NO: 112 - DNA, 17 bp, Homo sapiens
cgccaaagtgggccttt

SEQ ID NO: 113 - DNA, 22 bp, Homo sapiens
ccatgcaacagacctgctgccg

SEQ ID NO: 114 - protein, 18 aa, Artificial Sequence
  Synthesized Oligonucleotide.
MHKTIKKEIQDMLKKFFC

SEQ ID NO: 115 - DNA, 25 bp, Artificial Sequence
  Synthesized Oligonucleotide.
gugagacagguugcaggcucagugc

SEQ ID NO: 116 - DNA, 25 bp, Artificial Sequence
  Synthesized Oligonucleotide.
aaccagcacgguugagcggaugaug

SEQ ID NO: 117 - DNA, 25 bp, Artificial Sequence
  Synthesized Oligonucleotide.
gauccacugacaccacgacaauggu

SEQ ID NO: 118 - DNA, 25 bp, Artificial Sequence
  Synthesized Oligonucleotide.
ugaaggacaccacgcugagaauagu

SEQ ID NO: 119 - DNA, 25 bp, Artificial Sequence
  Synthesized Oligonucleotide.
ggaguagcaggcaaucaugacaauc

SEQ ID NO: 120 - DNA, 25 bp, Artificial Sequence
  Synthesized Oligonucleotide.
gctgactcggacgttggacagagtg

SEQ ID NO: 121 - DNA, 25 bp, Artificial Sequence
  Synthesized Oligonucleotide.
gtgtaagggcagttggcacgaccaa

SEQ ID NO: 122 - DNA, 25 bp, Artificial Sequence
  Synthesized Oligonucleotide.
ttggaacagcaccacagtcacctag

SEQ ID NO: 123 - DNA, 25 bp, Artificial Sequence
  Synthesized Oligonucleotide.
tgataagagtcgcaccacaggaagt

SEQ ID NO: 124 - DNA, 25 bp, Artificial Sequence
  Synthesized Oligonucleotide.
ctaacagtactaacggacgatgagg

SEQ ID NO: 125 - DNA, 21 bp, Homo sapiens
ctggagactctcagggtcgaa

SEQ ID NO: 126 - DNA, 17 bp, Homo sapiens
gcgcttccaggactgca

SEQ ID NO: 127 - DNA, 27 bp, Homo sapiens
acagatttctaccactccaaacgccgg

SEQ ID NO: 128 - DNA, 16 bp, Homo sapiens
cccggtggaccacgaa

SEQ ID NO: 129 - DNA, 19 bp, Homo sapiens
ggctcgcctcttccatgtc

SEQ ID NO: 130 - DNA, 25 bp, Homo sapiens
aacccgggacttggagaagcactgc

SEQ ID NO: 131 - DNA, 24 bp, Homo sapiens
gaggatgaggagagctatgacaca

SEQ ID NO: 132 - DNA, 22 bp, Homo sapiens
ccctttgcactcataacgtcag

SEQ ID NO: 133 - DNA, 29 bp, Homo sapiens
aaacacacagtcatcatagggcagctcgt

SEQ ID NO: 134 - DNA, 1071 bp, dna;Homo sapiens
atggctacaacagtccctgatggttgccgcaatggcctgaaatccaagtactacagactttgtgataaggctgaagcttggggcatcgtcctagaaacggtggccacagccggggttgtgacctcggtggccttcatgctcactctcccgatcctcgtctgcaaggtgcaggactccaacaggcgaaaaatgctgcctactcagtttctcttcctcctgggtgtgttgggcatctttggcctcaccttcgccttcatcatcggactggacgggagcacagggcccacacgcttcttcctctttgggatcctcttttccatctgcttctcctgcctgctggctcatgctgtcagtctgaccaagctcgtccgggggaggaagcccctttccctgttggtgattctgggtctggccgtgggcttcagcctagtccaggatgttatcgctattgaatatattgtcctgaccatgaataggaccaacgtcaatgtcttttctgagctttccgctcctcgtcgcaatgaagactttgtcctcctgctcacctacgtcctcttcttgatggcgctgaccttcctcatgtcctccttcaccttctgtggttccttcacgggctggaagagacatggggcccacatctacctcacgatgctcctctccattgccatctgggtggcctggatcaccctgctcatgcttcctgactttgaccgcaggtgggatgacaccatcctcagctccgccttggctgccaatggctgggtgttcctgttggcttatgttagtcccgagttttggctgctcacaaagcaacgaaaccccatggattatcctgttgaggatgctttctgtaaacctcaactcgtgaagaagagctatggtgtggagaacagagcctactctcaagaggaaatcactcaaggttttgaagagacaggggacacgctctatgccccctattccacacattttcagctgcagaaccagcctccccaaaaggaattctccatcccacgggcccacgcttggccgagcccttacaaagactatgaagtaaagaaagagggcagc

(19) United States
(12) Patent Application Publication
(10) Pub. No.: US 2004/0214317 A1
(43) Pub. Date: Oct. 28, 2004

(54) Novel human G-protein coupled receptor, HGPRBMY8, expressed highly in brain

(76) Inventors: Peter Battaglino, Prospect, CT (US); John N. Feder, Belle Mead, NJ (US); Gabriel Mintier, Hightstown, NJ (US); Thomas C. Nelson, Lawrenceville, NJ (US); Chandra S. Ramanathan, Wallingford, CT (US); Ryan Westphal, Cheshire, CT (US); Angela M. Cacace, Durham, CT (US); Lauren E. Barber, Higganum, CT (US); Donald R. Hawken, Trenton, NJ (US); Michael G. Kornacker, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/712,615

(22) Filed: Nov. 13, 2003

Related U.S. Application Data
(63) Continuation-in-part of application No. 09/992,238, filed on Nov. 14, 2001, now abandoned.
(60) Provisional application No. 60/248,285, filed on Nov. 14, 2000.
(60) Provisional application No. 60/268,581, filed on Feb. 14, 2001.
(60) Provisional application No. 60/308,285, filed on Jul. 27, 2001.
(60) Provisional application No. 60/317,166, filed on Sep. 4, 2001.

(51) Int. Cl. C12N 15/00

(52) U.S. Cl. 435/320.1

(57) ABSTRACT

The present invention describes a newly discovered human G-protein coupled receptor and its encoding polynucleotide. Also described are expression vectors, host cells, agonists, antagonists, antisense molecules, and antibodies associated with the polynucleotide and/or polypeptide of the present invention. In addition, methods for treating, diagnosing, preventing, and screening for disorders associated with aberrant cell growth, neurological conditions, and diseases or disorders related to the brain are illustrated.

This application is a continuation-in-part application of non-provisional application U.S. Ser. No. 09/992,238, filed Nov. 14, 2001, which claims benefit to provisional application U.S. Serial No. 60/248,285, filed Nov. 14, 2000; to provisional application U.S. Serial No. 60/268,581, filed Feb. 14, 2001; to provisional application U.S. Serial No. 60/308,285, filed Jul. 27, 2001; and to provisional application U.S. Serial No. 60/317,166, filed Sep. 4, 2001.

### FIELD OF THE INVENTION

The present invention relates to the fields of pharmacogenomics, diagnostics and patient therapy. More specifically, the present invention relates to methods of diagnosing and/or treating diseases involving the Human G-Protein Coupled Receptor, HGPRBMY8.

### BACKGROUND OF THE INVENTION

It is well established that many medically significant biological processes are mediated by proteins participating in signal transduction pathways that involve G-proteins and/or second messengers, e.g., cAMP (Lefkowitz, Nature, 351:353-354 (1991)). Herein these proteins are referred to as proteins participating in pathways with G-proteins or PPG proteins. Some examples of these proteins include the GPC receptors, such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding is activation of an enzyme, adenylate cyclase, inside the cell. Enzyme activation by hormones is dependent on the presence of the nucleotide GTP, and GTP also influences hormone binding. A G-protein connects the hormone receptors to adenylate cyclase. G-protein was shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane a-helices connected by extracellular or cytoplasmic loops. G-protein coupled receptors include a wide range of biologically active receptors, such as hormone, viral, growth factor and neuroreceptors.

G-protein coupled receptors have been characterized as including these seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxyl terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise a hydrophilic socket formed by several G-protein coupled receptors transmembrane domains, which socket is surrounded by hydrophobic residues of the G-protein coupled receptors. The hydrophilic side of each G-protein coupled receptor transmembrane helix is postulated to face inward and form the polar ligand-binding site. TM3 has been implicated in several G-protein coupled receptors as having a ligand-binding site, such as including the TM3 aspartate residue. Additionally, TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 10:317-331(1989)). Different G-protein β-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

G-protein coupled receptors (GPCRs) are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)). The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

Characterization of the HGPRBMY8 polypeptide of the present invention led to the determination that it is involved in the modulation of the p21 G1/S-phase cell cycle check point protein, either directly or indirectly; is involved in the modulation of the p27 G1/S-phase cell cycle check point protein, either directly or indirectly; and is involved in the NFkB pathway through modulation of the IkB protein, either directly or indirectly.

Critical transitions through the cell cycle are highly regulated by distinct protein kinase complexes, each composed of a cyclin regulatory and a cyclin-dependent kinase (cdk) catalytic subunit (for review see Draetta, Curr. Opin. Cell Biol. 6, 842-846 (1994)). These proteins regulate the cell's progression through the stages of the cell cycle and are, in turn, regulated by numerous proteins, including p53, p21, p16, and cdc25. Downstream targets of cyclin-cdk complexes include pRb and E2F. The cell cycle often is dysregulated in neoplasia due to alterations either in oncogenes that indirectly affect the cell cycle, or in tumor suppressor genes or oncogenes that directly impact cell cycle regulation, such as pRb, p53, p16, cyclin D1, or mdm-2 (for review see Schafer, Vet Pathol 1998 35, 461-478 (1998)).

P21, also known as CDNK1A (cyclin-dependent kinase inhibitor 1A), or CIP1 inhibits mainly the activity of cyclin CDK2 or CDK4 complexes. Therefore, p21 primarily blocks cell cycle progression at the G1 stage of the cell cycle. The expression of p21 is tightly controlled by the tumor suppressor protein p53, through which this protein mediates the cell cycle G1 phase arrest in response to a variety of stress stimuli. In addition, p21 protein interacts with the DNA polymerase accessory factor PCNA (proliferating cell nuclear antigen), and plays a regulatory role in S phase DNA replication and DNA damage repair.

After DNA damage, many cells appear to enter a sustained arrest in the G2 phase of the cell cycle. Bunz et al. (Science 282, 1497-1501 (1998)) demonstrated that this arrest could be sustained only when p53 was present in the cell and capable of transcriptionally activating the cyclin-dependent kinase inhibitor p21. After disruption of either the p53 or the p21 gene, gamma-radiated cells progressed into mitosis and exhibited a G2 DNA content only because of a failure of cytokinesis. Thus, p53 and p21 appear to be essential for maintaining the G2 cell cycle checkpoint in human cells.

Due to the connection between the transcriptional activity of p53 and p21 RNA expression, the readout of p21 RNA can be used to determine the effect of drugs or other insults (radiation, antisense for a specific gene, dominant negative expression) on a given cell system which contains wild type p53. Specifically, if a gene is removed using antisense products and this has an effect on the p53 activity, p21 will be upregulated and can serve therefore as an indirect marker for an influence on the cell cycle regulatory pathways and induction of cell cycle arrest.

In addition to cancer regulation of cell cycle activity has a role in numerous other systems. For example, hematopoietic stem cells are relative quiescent, while after receiving the required stimulus they undergo dramatic proliferation and inexorably move toward terminal differentiation. This is partly regulated by the presence of p21. Using p21 knockout mice Cheng et al. (Science 287, 1804-1808 (2000)) demonstrated its critical biologic importance in protecting the stem cell compartment. In the absence of p21, hematopoietic stem cell proliferation and absolute number were increased under normal homeostatic conditions. Exposing the animals to cell cycle-specific myelotoxic injury resulted in premature death due to hematopoietic cell depletion. Further, self-renewal of primitive cells was impaired in serially transplanted bone marrow from p21−/− mice, leading to hematopoietic failure. Therefore it was concluded that p21 is the molecular switch governing the entry of stem cells into the cell cycle, and in its absence, increased cell cycling leads to stem cell exhaustion. Under conditions of stress, restricted cell cycling is crucial to prevent premature stem cell depletion and hematopoietic death. Therefore, genes involved in the downregulation of p21 expression could have a stimulatory effect and therefore be useful for the exploration of stem cell technologies.

P27, also known as CDNK1B (cyclin-dependent kinase inhibitor 1B) or KIP1, shares a limited similarity with the CDK inhibitor CDKN1A/p21. The encoded protein binds to and prevents the activation of cyclinE-CDK2 or cyclinD-CDK4 complexes. Therefore it mainly blocks the cell cycle progression at the G1- and S-phases (for review see Desdouets and Brechot, Pathol Biol (Paris) 48, 203-210 (2000)).

Reduction in levels of p27 and increased expression of cyclin E also occur and carry a poor prognostic significance in many common forms of cancer. The inhibition of protein activities leading to an upregulation of p27 might therefore be a possibility to decrease the progression of cancer and increase patient survival rates (for review see Sgambato, et al., J Cell Physiol. 183, 18-27 (2000)).

Recently, Medema et al. (Nature 40, 782-787 (2000)) demonstrated that p27 is a major transcriptional target of forkhead transcription factors FKHRL1, AFX, or FKHR. Overexpression of these proteins causes growth suppression in a variety of cell lines, including a Ras-transformed cell line and a cell line lacking the tumor suppressor PTEN integrating signals from PI3K/PKB signaling and RAS/RAL signaling to regulate transcription of p27(KIP1). Expression of AFX blocked cell cycle progression at phase G1, independent of functional retinoblastoma protein but dependent on the cell cycle inhibitor p27(KIP1). This is further supported by the fact that AFX activity inhibits p27−/− knockout mouse cells significantly less than their p27+/+ counterparts.

The connection between the PTEN pathway and the activation of p27 via forkhead-like transcription factors implies that genes whose inhibition leads to p27 upregulation might be involved in this pathway. Therefore the identification of genes whose knockout leads to an upregulation of p27 might be useful drug targets, as inhibition of such genes should result in the upregulation of p27 and therefore be useful for the treatment and/or amelioration of cancer and increase a cancer patients prolonged outlook and survival.

The fate of a cell in multicellular organisms often requires choosing between life and death. This process of cell suicide, known as programmed cell death or apoptosis, occurs during a number of events in an organisms life cycle, such as for example, in development of an embryo, during the course of an immunological response, or in the demise of cancerous cells after drug treatment, among others. The final outcome of cell survival versus apoptosis is dependent on the balance of two counteracting events, the onset and speed of caspase cascade activation (essentially a protease chain reaction), and the delivery of antiapoptotic factors which block the caspase activity (Aggarwal B. B. Biochem. Pharmacol. 60, 1033-1039, (2000); Thornberry, N. A. and Lazebnik, Y. Science 281, 1312-1316, (1998)).

The production of antiapoptotic proteins is controlled by the transcriptional factor complex NF-kB. For example, exposure of cells to the protein tumor necrosis factor (TNF) can signal both cell death and survival, an event playing a major role in the regulation of immunological and inflammatory responses (Ghosh, S., May, M. J., Kopp, E. B. Annu. Rev. Immunol. 16, 225-260, (1998); Silverman, N. and Maniatis, T., Genes & Dev. 15, 2321-2342, (2001); Baud, V. and Karin, M., Trends Cell Biol. 11, 372-377, (2001)). The anti-apoptotic activity of NF-kB is also crucial to oncogenesis and to chemo- and radio-resistance in cancer (Baldwin, A. S., J. Clin. Inves. 107, 241-246, (2001)).

Nuclear Factor-kB (NF-kB), is composed of dimeric complexes of p50 (NF-kB1) or p52 (NF-kB2) usually associated with members of the Rel family (p65, c-Rel, Rel B) which have potent transactivation domains. Different combinations of NF-kB/Rel proteins bind distinct kB sites to regulate the transcription of different genes. Early work involving NF-kB suggested its expression was limited to specific cell types, particularly in stimulating the transcription of genes encoding kappa immunoglobulins in B lymphocytes. However, it has been discovered that NF-kB is, in fact, present and inducible in many, if not all, cell types and that it acts as an intracellular messenger capable of playing a broad role in gene regulation as a mediator of inducible signal transduction. Specifically, it has been demonstrated that NF-kB plays a central role in regulation of intercellular signals in many cell types. For example, NF-kB has been shown to positively regulate the human beta-interferon (beta-IFN) gene in many, if not all, cell types. Moreover, NF-kB has also been shown to serve the important function of acting as an intracellular transducer of external influences.

The transcription factor NF-kB is sequestered in an inactive form in the cytoplasm as a complex with its inhibitor, IkB, the most prominent member of this class being IkBa. A number of factors are known to serve the role of stimulators of NF-kB activity, such as, for example, TNF. After TNF exposure, the inhibitor is phosphorylated and proteolytically removed, releasing NF-kB into the nucleus and allowing its transcriptional activity. Numerous genes are upregulated by this transcription factor, among them IkBa. The newly synthezised IkBa protein inhibits NF-kB, effectively shutting down further transcriptional activation of its downstream effectors. However, as mentioned above, the IkBa protein may only inhibit NF-kB in the absence of IkBa stimuli, such as TNF stimulation, for example. Other agents that are known to stimulate NF-kB release, and thus NF-kB activity, are bacterial lipopolysaccharide, extracellular polypeptides, chemical agents, such as phorbol esters, which stimulate intracellular phosphokinases, inflammatory cytokines, IL-1, oxidative and fluid mechanical stresses, and Ionizing Radiation (Basu, S., Rosenzweig, K, R., Youmell, M., Price, B, D, Biochem, Biophys, Res, Commun., 247(1):79-83, (1998)). Therefore, as a general rule, the stronger the insulting stimulus, the stronger the resulting NF-kB activation, and the higher the level of IkBa transcription. As a consequence, measuring the level of IkBa RNA can be used as a marker for antiapoptotic events, and indirectly, for the onset and strength of pro- apoptotic events.

The upregulation of IkBa due to the downregulation of HGPRBMY8 places this GPCR protein into a signalling pathway potentially involved in apoptotic events. This gives the opportunity to regulate downstream events via the activity of the protein HGPRBMY8 with antisense polynucleotides, polypeptides or low molecular chemicals with the potential of achieving a therapeutic effect in cancer, autoimmune diseases. In addition to cancer and immunological disorders, NF-kB has significant roles in other diseases (Baldwin, A. S., J. Clin Invest. 107, :3-6 (2001)). NF-kB is a key factor in the pathophysiology of ischemia-reperfusion injury and heart failure (Valen, G., Yan. Z Q, Hansson, G K, J. Am. Coll. Cardiol. 38, 307-14 (2001)). Furthermore, NF-kB has been found to be activated in experimental renal disease (Guijarro C, Egido J., Kidney Int. 59, 415-425 (2001)).

The present invention provides a newly discovered G-protein coupled receptor protein, which may be involved in cellular growth properties in brain-related tissues based on its abundance found in the brain for this receptor. The present invention also relates to newly identified polynucleotides, polypeptides encoded by such polynucleotides, the use of such polynucleotides and polypeptides, as well as the production of such polynucleotides and polypeptides. More particularly, the polypeptides of the present invention are human 7-transmembrane receptors. The invention also relates to inhibiting the action of such polypeptides.

### SUMMARY OF THE INVENTION

The present invention provides a novel human member of the G-protein coupled receptor (GPCR) family (HGPRBMY8). Based on sequence homology, the protein HGPRBMY8 is a candidate GPCR. Based on its protein sequence information, the HGPRBMY8 contains seven transmembrane domains, which is a characteristic structural feature of GPCRs. The GPCR of this invention is closely related to the somatostatin and GPR24 receptor families based on sequence similarity using the BLAST program. This orphan GPCR is expressed highly in brain.

It is an object of the present invention to provide an isolated HGPRBMY8 polynucleotide as depicted in SEQ ID NO:1.

It is also an object of the present invention to provide the HGPRBMY8 polypeptide, encoded by the polynucleotide of SEQ ID NO:1 (CDS=1 to 1524) and having the amino acid sequence of SEQ ID NO:2, or a functional or biologically active portion thereof.

It is a further object of the present invention to provide compositions comprising the HGPRBMY8 polynucleotide sequence, or a fragment thereof, or the encoded HGPRBMY8 polypeptide (MW=56.7 Kd), or a fragment or portion thereof. Also provided by the present invention are pharmaceutical compositions comprising at least one HGPRBMY8 polypeptide, or a functional portion thereof, wherein the compositions further comprise a pharmaceutically acceptable carrier, excipient, or diluent.

It is an object of the present invention to provide a novel, isolated, and substantially purified polynucleotide that encodes the HGPRBMY8 GPCR homologue, or fragment thereof. In a particular aspect, the polynucleotide comprises the nucleotide sequence of SEQ ID NO:1. The present invention also provides a polynucleotide sequence comprising the complement of SEQ ID NO:1, or variants thereof. In addition, the present invention features polynucleotide sequences, which hybridize under conditions of moderate stringency or high stringency to the polynucleotide sequence of SEQ ID NO:1.

It is an object of the present invention to further provide a nucleic acid sequence encoding the HGPRBMY8 polypeptide and an antisense of the nucleic acid sequence, as well as oligonucleotides, fragments, or portions of the nucleic acid molecule or antisense molecule. Also provided are expression vectors and host cells comprising polynucleotides that encode the HGPRBMY8 polypeptide.

It is an object of the invention to provide methods for producing a polypeptide comprising the amino acid sequence depicted in SEQ ID NO:2, or a fragment thereof, comprising the steps of a) cultivating a host cell containing an expression vector containing at least a functional fragment of the polynucleotide sequence encoding the HGPRBMY8 protein according to this invention under conditions suitable for the expression of the encoded polypeptide; and b) recovering the polypeptide from the host cell.

It is also an object of the invention to provide antibodies, and binding fragments thereof, which bind specifically to the HGPRBMY8 polypeptide, or an epitope thereof, for use as therapeutic and diagnostic agents.

It is a further object of the invention to provide methods for screening for agents which bind to, or modulate HGPRBMY8 polypeptide, e.g., agonists and antagonists, as well as the binding molecules and/ or modulators, e.g., agonists and antagonists, particularly those that are obtained from the screening methods described.

It is an object of the present invention to also provide a substantially purified antagonist or inhibitor of the polypeptide of SEQ ID NO:2. In this regard, and by way of example, a purified antibody that binds to a polypeptide comprising the amino acid sequence of SEQ ID NO:2 is provided.

It is an object of the invention to further provide substantially purified agonists or activators of the polypeptide of SEQ ID NO:2 are further provided.

It is another object of the present invention to provide HGPRBMY8 nucleic acid sequences, polypeptide, peptides and antibodies for use in the diagnosis and/or screening of disorders or diseases associated with expression of the polynucleotide and its encoded polypeptide as described herein.

It is a also an object of the present invention to provide kits for screening and diagnosis of disorders associated with aberrant or uncontrolled cellular development and with the expression of the polynucleotide and its encoded polypeptide as described herein.

It is an object of the present invention to further provide methods for the treatment or prevention of cancers, immune disorders, or neurological disorders involving administering to an individual in need of treatment or prevention an effective amount of a purified antagonist of the HGPRBMY8 polypeptide. Due to its elevated expression in brain, the novel GPCR protein of the present invention is particularly useful in treating or preventing neurological disorders, conditions, or diseases.

It is an object of the present invention to also provide a method for detecting a polynucleotide that encodes a G-protein coupled receptor, preferably the HGPRBMY8 polypeptide, or homologue, or fragment thereof, in a biological sample comprising the steps of: a) hybridizing the polynucleotide, or complement of the polynucleotide sequence encoding SEQ ID NO:2 to a nucleic acid material of a biological sample, thereby forming a hybridization complex; and b) detecting the hybridization complex, wherein the presence of the complex correlates with the presence of a polynucleotide encoding the HGPRBMY8 polypeptide, or fragment thereof, in the biological sample. The nucleic acid material may be further amplified by the polymerase chain reaction prior to hybridization.

It is an object of the instant invention to provide methods and compositions to detect and diagnose alterations in the HGPRBMY8 sequence in tissues and cells as they relate to ligand response.

It is an object of the present invention to further provide compositions for diagnosing brain-related disorders and for diagnosing or monitoring response to HGPRBMY8 therapy in humans. In accordance with the invention, the compositions detect an alteration of the normal or wild type HGPRBMY8 sequence or its expression product in a patient sample of cells or tissue.

It is an object of the present invention to provide diagnostic probes for diseases and a patient's response to therapy. The probe sequence comprises the HGPRBMY8 locus polymorphism. The probes can be constructed of nucleic acids or amino acids.

It is an object of the present invention to further provide antibodies, and immunoreactive portions thereof, that recognize and bind to the HGPRBMY8 protein. Such antibodies can be either polyclonal or monoclonal. Antibodies that bind to the HGPRBMY8 protein can be utilized in a variety of diagnostic and prognostic formats and therapeutic methods.

It is also an object of the present invention to provide diagnostic kits for the determination of the nucleotide sequence of human HGPRBMY8 alleles. The kits are based on amplification-based assays, nucleic acid probe assays, protein nucleic acid probe assays, antibody assays or any combination thereof.

It is an object of the instant invention to further provide methods for detecting genetic predisposition, susceptibility and response to therapy related to the brain. In accordance with the invention, the method comprises isolating a human sample, for example, blood or tissue from adults, children, embryos or fetuses, and detecting at least one alteration in the wild type HGPRBMY8 sequence, or its expression product, from the sample, wherein the alterations are indicative of genetic predisposition, susceptibility or altered response to therapy related to the brain.

The invention further relates to a method for preventing, treating, or ameliorating a medical condition with the polypeptide provided as SEQ ID NO:2, in addition to, its encoding nucleic acid, or a modulator thereof, wherein the medical condition is a neurological disorder; nucleus accumbens disorders, caudate nucleus disorders, neurotransmitter disorders, neurotransmitter expression disorders, neurotransmitter release disorders, disorders associated with aberrant dopamine synthesis, disorders associated with aberrant dopamine release, disorders associated with aberrant dopamine function, disorders associated with aberrant opioid peptide synthesis, disorders associated with aberrant opioid peptide release, disorders associated with aberrant opioid peptide function, disorders associated with aberrant serotonin synthesis, disorders associated with aberrant serotonin release, disorders associated with aberrant serotonin function, disorders associated with aberrant GABA synthesis, disorders associated with aberrant GABA release, disorders associated with aberrant GABA function, disorders associated with the release of GABA from L-glutamic acid, schizophrenia, Parkinson's disease, progressive supranuclear palsy, Alzheimer's, affective disorders, depression, aggressive behavioral disorders, addictive disorders, sleep disorders, eating disorders neuropathic pain; psychotic disorders; fear, stress disorders, severe mental retardation; dyskinesias, Huntington's disease; Gilles dela Tourett's syndrome; Sydenham chorea; major depressive disorder; obsessive-compulsive disorder; movement type disorders; anxiety; schizophrenia; manic depression; delirium; dementia; brain cancer, proliferative disorder of the brain, and neoplastic diseases of the brain.

The invention further relates to a method of diagnosing a pathological condition or a susceptibility to a pathological condition in a subject comprising the steps of (a) determining the presence or amount of expression of the polypeptide of of SEQ ID NO:2 in a biological sample; (b) and diagnosing a pathological condition or a susceptibility to a pathological condition based on the presence or amount of expression of the polypeptide relative to a control, wherein said condition is a member of the group consisting of a neurological disease state, including a neurological disorder; nucleus accumbens disorders, caudate nucleus disorders, neurotransmitter disorders, neurotransmitter expression disorders, neurotransmitter release disorders, disorders associated with aberrant dopamine synthesis, disorders associated with aberrant dopamine release, disorders associated with aberrant dopamine function, disorders associated with aberrant opioid peptide synthesis, disorders associated with aberrant opioid peptide release, disorders associated with aberrant opioid peptide function, disorders associated with aberrant serotonin synthesis, disorders associated with aberrant serotonin release, disorders associated with aberrant serotonin function, disorders associated with aberrant GABA synthesis, disorders associated with aberrant GABA release, disorders associated with aberrant GABA function, disorders associated with the release of GABA from L-glutamic acid, schizophrenia, Parkinson's disease, progressive supranuclear palsy, Alzheimer's, affective disorders, depression, aggressive behavioral disorders, addictive disorders, sleep disorders, eating disorders neuropathic pain; psychotic disorders; fear, stress disorders, severe mental retardation; dyskinesias, Huntington's disease; Gilles dela Tourett's syndrome; Sydenham chorea; major depressive disorder; obsessive-compulsive disorder; movement type disorders; anxiety; schizophrenia; manic depression; delirium; dementia; brain cancer, proliferative disorder of the brain, and neoplastic diseases of the brain.

The invention further relates to a method for preventing, treating, or ameliorating a medical condition with the polypeptide provided as SEQ ID NO:2, in addition to, its encoding nucleic acid, or a modulator thereof, wherein the medical condition is an anxiolytic disorder.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant p21 expression or activity.

The invention further relates to a method for preventing, treating, or ameliorating a medical condition with the polypeptide provided as SEQ ID NO:2, in addition to, its encoding nucleic acid, or a modulator thereof, wherein the medical condition is a cell cycle defect, disorders related to aberrant phosphorylation, disorders related to aberrant signal transduction, proliferating disorders, and/or cancers.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant cell cycle regulation.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant cell cycle regulation.

The invention further relates to a method of increasing, or alternatively decreasing, the number of cells in the G1 phase of the cell cycle comprising the step of administering an antagonist, or alternatively an agonist, of HGPRBMY8.

The invention further relates to a method of increasing, or alternatively decreasing, the number of cells in the G2 phase of the cell cycle comprising the step of administering an antagonist, or alternatively an agonist, of HGPRBMY8.

The invention further relates to a method of decreasing, or alternatively increasing, the number of cells that progress to the S phase of the cell cycle comprising the step of administering an antagonist, or alternatively an agonist, of HGPRBMY8.

The invention further relates to a method of increasing, or alternatively decreasing, the number of cells that progress to the M phase of the cell cycle comprising the step of administering an antagonist, or alternatively an agonist, of HGPRBMY8.

The invention further relates to a method of inducing, or alternatively inhibiting, cells into G1 and/or G2 phase arrest comprising the step of administering an antagonist, or alternatively an agonist, of HGPRBMY8.

The invention also relates to an antisense compound 8 to 30 nucleotides in length that specifically hybridizes to a nucleic acid molecule encoding the human HGPRBMY8 polypeptide of the present invention, wherein wherein said antisense compound inhibits the expression of the human HGPRBMY8 polypeptide.

The invention further relates to a method of inhibiting the expression of the human HGPRBMY8 polypeptide of the present invention in human cells or tissues comprising contacting said cells or tissues in vitro, or in vivo, with an antisense compound of the present invention so that expression of the HGPRBMY8 polypeptide is inhibited.

The invention further relates to a method of increasing, or alternatively decreasing, the expression of p21 in human cells or tissues comprising contacting said cells or tissues in vitro, or in vivo, with an antisense compound that specifically hybridizes to a nucleic acid molecule encoding the human HGPRBMY8 polypeptide of the present invention so that expression of the HGPRBMY8 polypeptide is inhibited.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant p27 expression or activity.

The invention further relates to a method of increasing, or alternatively decreasing, the expression of p27 in human cells or tissues comprising contacting said cells or tissues in vitro, or in vivo, with an antisense compound that specifically hybridizes to a nucleic acid molecule encoding the human HGPRBMY8 polypeptide of the present invention so that expression of the HGPRBMY8 polypeptide is inhibited.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant IkB expression or activity, or a disorder associated with aberrant NFkB expression or activity.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with aberrant apoptosis.

The invention further relates to a method for preventing, treating, or ameliorating a medical condition with the polypeptide provided as SEQ ID NO:2, in addition to, its encoding nucleic acid, or a modulator thereof, wherein the medical condition is a proliferative disorder, a cancer, ischemia-reperfusion injury, heart failure, immuno compromised conditions, renal diseases, ameliorating autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, HIV, and HIV propagation in cells infected with other viruses.

The present invention is also directed to a method of identifying a compound that modulates the biological activity of HGPRBMY8, comprising the steps of, (a) combining a candidate modulator compound with HGPRBMY8 in the presence of an antisense molecule that antagonizes the activity of the HGPRBMY8 polypeptide selected from the group consisting of SEQ ID NO:115 to 124, and (b) identifying candidate compounds that reverse the antagonizing effect of the peptide.

The present invention is also directed to a method of identifying a compound that modulates the biological activity of HGPRBMY8, comprising the steps of, (a) combining a candidate modulator compound with HGPRBMY8 in the presence of a small molecule that antagonizes the activity of the HGPRBMY8 polypeptide selected from the group consisting of SEQ ID NO:115 to 124, and (b) identifying candidate compounds that reverse the antagonizing effect of the peptide.

The present invention is also directed to a method of identifying a compound that modulates the biological activity of HGPRBMY8, comprising the steps of, (a) combining a candidate modulator compound with HGPRBMY8 in the presence of a small molecule that agonizes the activity of the HGPRBMY8 polypeptide selected from the group consisting of SEQ ID NO:115 to 124, and (b) identifying candidate compounds that reverse the agonizing effect of the peptide.

It is an additional object of the present invention to provide methods for making determinations as to which drug to administer, dosages, duration of treatment and the like.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are HEK cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, and futher wherein said cells express the polypeptide at either low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said cells express beta lactamase at low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit HGPRBMY8, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said cells express beta lactamase at low, moderate, or high levels.

The statement, “wherein said cells express beta lactamase at low, moderate, or high levels” is a reference to cells that either express beta lactamase at low, moderate, or high levels relative to the expression levels of a reference mRNA, gene, or protein; or a reference to the actual percentage of cells that express beta lactamase. In the latter example, high levels of expression would be achieved if the majority of cells were expressing beta lactamase, while low levels of expression would be achieved if only a subset of cells were expressing beta lactamase. Such cells may also express other proteins, such as the proteins of the present invention at low, moderate, or high levels as well.

Further objects, features, and advantages of the present invention will be better understood upon a reading of the detailed description of the invention when considered in connection with the accompanying figures/drawings.

### BRIEF DESCRIPTION OF THE FIGURES

FIG. 1 shows the full-length nucleotide sequence of cDNA clone HGPRBMY8, a human G-protein coupled receptor (SEQ ID NO:1).

FIG. 2 shows the amino acid sequence (SEQ ID NO:2) from the translation of the full-length HGPRBMY8 cDNA sequence.

FIG. 3 shows the 5′ untranslated sequence of the orphan HGPRBMY8 (SEQ ID NO:3).

FIG. 4 shows the 3′ untranslated sequence of the orphan HGPRBMY8 (SEQ ID NO:4).

FIG. 5 shows the predicted transmembrane region of the HGPRBMY8 protein where the predicted transmembrane regions, represented by bold-faced and underlined type, correspond to the peaks with scores above 1500.

FIGS. 6A-6J show the multiple sequence alignment of the translated sequence of the orphan G-protein coupled receptor, HGPRBMY8, where the GCG (Genetics Computer Group) pileup program was used to generate the alignment with several known adrenergic and serotonin receptor sequences. The blackened areas represent identical amino acids in more than half of the listed sequences and the grey highlighted areas represent similar amino acids. As shown in FIGS. 6A-6J, the sequences are aligned according to their amino acids, where: HGPRBMY8 (SEQ ID NO:2) is encoded by full length HGPRBMY8 cDNA; ACM4_CHICK (SEQ ID NO:7) represents the Gallus gallus (chicken)form of muscarinic acetylcholine receptor M4; YDBM_CAEEL (SEQ ID NO:8) is the Caenorhabditis elegans form of an orphan GPCR; 5H1A_HUMAN (SEQ ID NO:9) is the human form of the 5HT-1A receptor; 5H1A_MOUSE (SEQ ID NO:10) is the Mus musculus (house mouse) form of the 5HT-1A receptor; 5H1A_FUGRU (SEQ ID NO:11) represents the Fugu rubripes form of the 5HT-1A receptor; 5HT_LYMST (SEQ ID NO:12) is the Lymnaea stagnalis (great pond snail) form of the 5HT-1A receptor; A1AD_HUMAN (SEQ ID NO:13) is the human form of the alpha-1D adrenergic receptor; A1AD_MOUSE (SEQ ID NO:14) represents the mouse form of the alpha-1D adrenergic receptor (alpha 1D-adrenoceptor); Q13675 (SEQ ID NO:15) is the human form of the alpha 1C adrenergic receptor isoform 2; Q13729 (SEQ ID NO:16) represents the human form of the alpha 1C adrenergic receptor isoform 3; 060451 is the human form of the alpha 1A adrenergic receptor isoform 4 (SEQ ID NO:17); A1AA_RAT (SEQ ID NO:18) is the Rattus norvegicus (Norway rat) form of the alpha-1A adrenergic receptor; 054913 (SEQ ID NO:19) is the Mus musculus (house mouse) form of the alpha 1A-adrenergic receptor; A1AA_BOVIN (SEQ ID NO:20) represents the Bos taurus (bovine) form of the alpha-1A adrenergic receptor; A1AA_CANFA (SEQ ID NO:21) is the Canis familiaris (dog) form of the alpha-1A adrenergic receptor; A1AA_RABIT (SEQ ID NO:22) represents the Oryctolagus cuniculus (rabbit) form of the alpha-1A adrenergic receptor; A1AA_HUMAN (SEQ ID NO:23) is the human form of the alpha-1A adrenergic receptor; A1AA_ORYLA (SEQ ID NO:24) is the Oryzias latipes japanese medaka) form of the alpha-1A adrenergic receptor (MAR1); and 096716 (SEQ ID NO:25) represents the Branchiostoma lanceolatum (amphioxus) form of the dopamine D1/beta receptor; and 075963 (SEQ ID NO:40) is the human form of the G-protein coupled receptor RE2.

FIG. 7 shows the expression profiling of the novel human orphan GPCR, HGPRBMY8, as described in Example 3.

FIG. 8 shows the brain-specific expression profiling of the novel human orphan GPCR, HGPRBMY8, as described in Example 4.

FIG. 9 shows the multiple sequence alignment of HGPRBMY8 and other potential SNP variants (amino acid alignment). The blackened areas represent identical amino acids and the grey highlighted areas represent similar amino acids. As shown in FIG. 9, the sequences are aligned according to their amino acids, where: AL390879 (SEQ ID NO:41), AX148250 (SEQ ID NO:42), and AX080495 (SEQ ID NO:43) are compared to HGPRBMY8 (SEQ ID NO:2).

FIGS. 10A-D shows the multiple sequence alignment of HGPRBMY8 and other potential SNP variants (nucleic acid alignment). The blackened areas represent identical amino acids and the grey highlighted areas represent similar amino acids. As shown in FIG. 10, the sequences are aligned according to their nucleic acids, where: AX080495 (SEQ ID NO:44); AL390879 (SEQ ID NO:45), AX148250 (SEQ ID NO:46), and are compared to HGPRBMY8 (SEQ ID NO:47).

FIG. 11 shows the FACS profile of an untransfected CHO-NFAT/CRE cell line.

FIG. 12 shows that overexpression of HGPRBMY8 constitutively couples through the NFAT/CRE Response Element.

FIG. 13 shows the FACS profile for the untransfected cAMP Response Element.

FIG. 14 shows the overexpression of HGPRBMY8 results in coupling through the cAMP Response Element.

FIGS. 15A-B shows the localization of expressed HGPRBMY8 to the cell surface.

FIGS. 16A-D shows representative transfected CHO-NFAT/CRE cell lines with intermediate and high beta lactamase expression levels useful in screens to identify HGPRBMY8 agonists and/or antagonists.

FIG. 17 shows the expression profile of the novel human orphan GPCR, HGPRBMY8, as described in Example 8 and Table 1.

FIGS. 18A-B show the polynucleotide sequence (SEQ ID NO:48) and deduced amino acid sequence (SEQ ID NO:49) of the human G-protein coupled receptor, HGPRBMY8, comprising, or alternatively consisting of, one or more of the predicted polynucleotide polymorphic loci, in addition to, the encoded polypeptide polymorphic loci of the present invention for this particular protein.

FIG. 19 shows an expanded expression profile of the novel human G-protein coupled receptor, HGPRBMY8. The figure illustrates the relative expression level of HGPRBMY8 amongst various mRNA tissue sources. As shown, the HGPRBMY8 polypeptide was expressed predominately in the nervous system, with predominate expression in the nucleus accumbens. Expression of HGPRBMY8 was also significantly expressed in other brain sub-regions, including the hypothalamus, amygdala, and to a lesser extent in the hippocampus, caudate, cerebellum, raphe nucleus, the locus coeruleus, and the medulla oblongata. Expression data was obtained by measuring the steady state HGPRBMY8 mRNA levels by quantitative PCR using the PCR primer pair provided as SEQ ID NO:105 and 106, and Taqman probe (SEQ ID NO:107) as described in Example 15 herein.

FIG. 20 shows a comparison of the expression profile of the novel human G-protein coupled receptor, HGPRBMY8, with another G-protein coupled receptor, HGPRBMY34 (SEQ ID NO:103; Co-pending U.S. Ser. No. 10/314,076, filed Dec. 6, 2002; U.S. Publication No. US20030152977A1, published August 14^{th}, 2003; which is hereby incorporated by reference herein in its entirety), in addition to glutamate decarboxylase (SEQ ID NO:104; Genbank Accession No. NM_{—}000818). As shown, the expression profile of the HGPRBMY8 polypeptide is identical to the expression profile of HGPRBMY34, suggesting the expression of HGPRBMY8 and HGPRBMY34 are co-regulated, that this co-expression in physiologically relevant, and that both GPCRs may form heterodimers. Additionally, the expression pattern of HGPRBMY8 shares significant similarity to glutamate decarboxylase. Expression data was obtained by measuring the steady state HGPRBMY8, HGPRBMY34, and glutamate decarboxylase mRNA levels by quantitative PCR using the PCR primer pair provided as SEQ ID NO:105 and 106, and Taqman probe (SEQ ID NO:107) for HGPRBMY8, the PCR primer pair provided as SEQ ID NO:108 and 109, and Taqman probe (SEQ ID NO:110) for HGPRBMY34, and the PCR primer pair provided as SEQ ID NO:111 and 112, and Taqman probe (SEQ ID NO:113) for glutamate decarboxylase, as described in Example 15 herein.

FIGS. 21 and 22 show a comparison of the ATP responses of HGPRBMY8- and Control cDNA-Transfected Cells observed in the screening method described in Example 18. Both FIGS. 21 and 22 show the results of an overlay of the calcium imaging data obtained from representative screening plates (baseline corrected) for HEK293 cells transiently transfected with either HGPRBMY8 (FIG. 21) or a control oGPCR cDNA (FIG. 22) after stimulation with 100 μM ATP.

### DETAILED DESCRIPTION OF THE PRESENT INVENTION

The present invention provides a novel isolated polynucleotide and encoded polypeptide, the expression of which is high in brain. This novel polypeptide is termed herein HGPRBMY8, an acronym for “Human G-Protein coupled Receptor HGPRBMY8”. HGPRBMY8 is also referred to as GPCR58 and GPCR84.

### Definitions

The HGPRBMY8 polypeptide (or protein) refers to the amino acid sequence of substantially purified HGPRBMY8, which may be obtained from any species, preferably mammalian, and more preferably, human, and from a variety of sources, including natural, synthetic, semi-synthetic, or recombinant. Functional fragments of the HGPRBMY8 polypeptide are also embraced by the present invention.

An “agonist” refers to a molecule which, when bound to the HGPRBMY8 polypeptide, or a functional fragment thereof, increases or prolongs the duration of the effect of the HGPRBMY8 polypeptide. Agonists may include proteins, nucleic acids, carbohydrates, or any other molecules that bind to and modulate the effect of HGPRBMY8 polypeptide. An antagonist refers to a molecule which, when bound to the HGPRBMY8 polypeptide, or a functional fragment thereof, decreases the amount or duration of the biological or immunological activity of HGPRBMY8 polypeptide. “Antagonists” may include proteins, nucleic acids, carbohydrates, antibodies, or any other molecules that decrease or reduce the effect of HGPRBMY8 polypeptide.

“Nucleic acid sequence”, as used herein, refers to an oligonucleotide, nucleotide, or polynucleotide, and fragments or portions thereof, and to DNA or RNA of genomic or synthetic origin which may be single- or double-stranded, and represent the sense or anti-sense strand. By way of non-limiting example, fragments include nucleic acid sequences that are greater than 20-60 nucleotides in length, and preferably include fragments that are at least 70-100 nucleotides, or which are at least 1000 nucleotides or greater in length.

Similarly, “amino acid sequence” as used herein refers to an oligopeptide, peptide, polypeptide, or protein sequence, and fragments or portions thereof, and to naturally occurring or synthetic molecules. Amino acid sequence fragments are typically from about 5 to about 30, preferably from about 5 to about 15 amino acids in length and retain the biological activity or function of the HGPRBMY8 polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. In addition, the terms HGPRBMY8 polypeptide and HGPRBMY8 protein are used interchangeably herein to refer to the encoded product of the HGPRBMY8 nucleic acid sequence of the present invention.

A “variant” of the HGPRBMY8 polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “non-conservative” changes, e.g., replacement of a glycine with a tryptophan. Minor variations may also include amino acid deletions or insertions, or both. Guidance in determining which amino acid residues may be substituted, inserted, or deleted without abolishing functional biological or immunological activity may be found using computer programs well known in the art, for example, DNASTAR software.

An “allele” or “allelic sequence” is an alternative form of the HGPRBMY8 nucleic acid sequence. Alleles may result from at least one mutation in the nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Altered” nucleic acid sequences encoding HGPRBMY8 polypeptide include nucleic acid sequences containing deletions, insertions and/or substitutions of different nucleotides resulting in a polynucleotide that encodes the same or a functionally equivalent HGPRBMY8 polypeptide. Altered nucleic acid sequences may further include polymorphisms of the polynucleotide encoding the HGPRBMY8 polypeptide; such polymorphisms may or may not be readily detectable using a particular oligonucleotide probe. The encoded protein may also contain deletions, insertions, or substitutions of amino acid residues, which produce a silent change and result in a functionally equivalent HGPRBMY8 protein. Deliberate amino acid substitutions may be made on the basis of similarity in polarity, charge, solubility, hydrophobicity, hydrophilicity, and/or the amphipathic nature of the residues, as long as the biological activity of HGPRBMY8 protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers” refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

“Amplification” refers to the production of additional copies of a nucleic acid sequence and is generally carried out using polymerase chain reaction (PCR) technologies, which are well known and practiced in the art (see, D.W. Dieffenbach and G. S. Dveksler, 1995, PCR Primer, a Laboratory Manual, Cold Spring Harbor Press, Plainview, N.Y.).

“Microarray” is an array of distinct polynucleotides or oligonucleotides synthesized on a substrate, such as paper, nylon, or other type of membrane; filter; chip; glass slide; or any other type of suitable solid support.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNA and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

The term “consensus” refers to the sequence that reflects the most common choice of base or amino acid at each position among a series of related DNA, RNA or protein sequences. Areas of particularly good agreement often represent conserved functional domains.

A “deletion” refers to a change in either nucleotide or amino acid sequence and results in the absence of one or more nucleotides or amino acid residues. By contrast, an insertion (also termed “addition”) refers to a change in a nucleotide or amino acid sequence that results in the addition of one or more nucleotides or amino acid residues, as compared with the naturally occurring molecule. A substitution refers to the replacement of one or more nucleotides or amino acids by different nucleotides or amino acids.

A “derivative” nucleic acid molecule refers to the chemical modification of a nucleic acid encoding, or complementary to, the encoded HGPRBMY8 polypeptide. Such modifications include, for example, replacement of hydrogen by an alkyl, acyl, or amino group. A nucleic acid derivative encodes a polypeptide, which retains the essential biological and/or functional characteristics of the natural molecule. A derivative polypeptide is one, which is modified by glycosylation, pegylation, or any similar process that retains the biological and/or functional or immunological activity of the polypeptide from which it is derived.

The term “biologically active”, i.e., functional, refers to a protein or polypeptide or fragment thereof having structural, regulatory, or biochemical functions of a naturally occurring molecule. Likewise, “immunologically active” refers to the capability of the natural, recombinant, or synthetic HGPRBMY8, or any oligopeptide thereof, to induce a specific immune response in appropriate animals or cells, for example, to generate antibodies, and to bind with specific antibodies.

The term “hybridization” refers to any process by which a strand of nucleic acid binds with a complementary strand through base pairing.

The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (e.g., Cₒt or Rₒt analysis), or between one nucleic acid sequence present in solution and another nucleic acid sequence immobilized on a solid support (e.g., membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (e.g., formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency that is different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511). As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, e.g., high, moderate, or low stringency, typically relates to such washing conditions.

Thus, by way of non-limiting example, “high stringency” refers to conditions that permit hybridization of those nucleic acid sequences that form stable hybrids in 0.018M NaCl at about 65° C. (i.e., if a hybrid is not stable in 0.018M NaCl at about 65° C., it will not be stable under high stringency conditions). High stringency conditions can be provided, for instance, by hybridization in 50% formamide, 5× Denhardt's solution, 5×SSPE (saline sodium phosphate EDTA) (1×SSPE buffer comprises 0.15 M NaCl, 10 mM Na₂HPO₄, 1 mM EDTA), (or 1×SSC buffer containing 150 mM NaCl, 15 mM Na₃ citrate·2H₂O, pH 7.0), 0.2% SDS at about 42° C., followed by washing in 1×SSPE (or saline sodium citrate, SSC) and 0.1% SDS at a temperature of at least about 42° C., preferably about 55° C., more preferably about 65° C.

“Moderate stringency” refers, by non-limiting example, to conditions that permit hybridization in 50% formamide, 5× Denhardt's solution, 5×SSPE (or SSC), 0.2% SDS at 42° C. (to about 50° C.), followed by washing in 0.2×SSPE (or SSC) and 0.2% SDS at a temperature of at least about 42° C., preferably about 55° C., more preferably about 65° C.

“Low stringency” refers, by non-limiting example, to conditions that permit hybridization in 10% formamide, 5× Denhardt's solution, 6×SSPE (or SSC), 0.2% SDS at 42° C., followed by washing in 1×SSPE (or SSC) and 0.2% SDS at a temperature of about 45° C., preferably about 50° C.

For additional stringency conditions, see T. Maniatis et al., Molecular Cloning: A Laboratory Manual, Cold Spring Harbor Laboratory, Cold Spring Harbor, N.Y. (1982). It is to be understood that the low, moderate and high stringency hybridization/washing conditions may be varied using a variety of ingredients, buffers and temperatures well known to and practiced by the skilled artisan.

The terms “complementary” or “complementarity” refer to the natural binding of polynucleotides under permissive salt and temperature conditions by base pairing. For example, the sequence “A-G-T” binds to the complementary sequence “T-C-A”. Complementarity between two single-stranded molecules may be “partial”, in which only some of the nucleic acids bind, or it may be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to using the functional term “substantially homologous”. The inhibition of hybridization of the completely complementary sequence to the target sequence may be examined using a hybridization assay (e.g., Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

Those having skill in the art will know how to determine percent identity between or among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

A “composition” comprising a given polynucleotide sequence refers broadly to any composition containing the given polynucleotide sequence. The composition may comprise a dry formulation or an aqueous solution. Compositions comprising polynucleotide sequence (SEQ ID NO:1) encoding HGPRBMY8 polypeptide (SEQ ID NO:2), or fragments thereof, may be employed as hybridization probes. The probes may be stored in freeze-dried form and may be in association with a stabilizing agent such as a carbohydrate. In hybridizations, the probe may be employed in an aqueous solution containing salts (e.g., NaCl), detergents or surfactants (e.g., SDS) and other components (e.g., Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% or greater free from other components with which they are naturally associated.

The term “sample”, or “biological sample”, is meant to be interpreted in its broadest sense. A biological sample suspected of containing nucleic acid encoding HGPRBMY8 protein, or fragments thereof, or HGPRBMY8 protein itself, may comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “mimetic” refers to a molecule, the structure of which is developed from knowledge of the structure of HGPRBMY8 protein, or portions thereof, and as such, is able to effect some or all of the actions of HGPRBMY8 protein.

The term “portion” with regard to a protein (as in “a portion of a given protein”) refers to fragments or segments of that protein. The fragments may range in size from four or five amino acid residues to the entire amino acid sequence minus one amino acid. Thus, a protein “comprising at least a portion of the amino acid sequence of SEQ ID NO: 2” encompasses the full-length human HGPRBMY8 polypeptide, and fragments thereof.

The term “antibody” refers to intact molecules as well as fragments thereof, such as Fab, F(ab′)₂, Fv, or Fc, which are capable of binding an epitopic or antigenic determinant. Antibodies that bind to HGPRBMY8 polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g, a mouse, a rat, or a rabbit).

The term “humanized” antibody refers to antibody molecules in which amino acids have been replaced in the non-antigen binding regions in order to more closely resemble a human antibody, while still retaining the original binding capability, e.g., as described in U.S. Pat. No. 5,585,089 to C. L. Queen et al.

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The term “correlates with expression of a polynucleotide” indicates that the detection of the presence of ribonucleic acid that is similar to SEQ ID NO:1 by Northern analysis is indicative of the presence of mRNA encoding HGPRBMY8 polypeptide (SEQ ID NO:2) in a sample and thereby correlates with expression of the transcript from the polynucleotide encoding the protein.

An alteration in the polynucleotide of SEQ ID NO:1 comprises any alteration in the sequence of the polynucleotides encoding HGPRBMY8 polypeptide, including deletions, insertions, and point mutations that may be detected using hybridization assays. Included within this definition is the detection of alterations to the genomic DNA sequence which encodes HGPRBMY8 polypeptide (e.g., by alterations in the pattern of restriction fragment length polymorphisms capable of hybridizing to SEQ ID NO:1), the inability of a selected fragment of SEQ ID NO:1 to hybridize to a sample of genomic DNA (e.g., using allele-specific oligonucleotide probes), and improper or unexpected hybridization, such as hybridization to a locus other than the normal chromosomal locus for the polynucleotide sequence encoding HGPRBMY8 polypeptide (e.g., using fluorescent in situ hybridization (FISH) to metaphase chromosome spreads).

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

### DESCRIPTION OF THE PRESENT INVENTION

The present invention provides a novel human member of the G-protein coupled receptor (GPCR) family (HGPRBMY8). Based on sequence homology, the protein HGPRBMY8 is a novel human GPCR. This protein sequence has been predicted to contain seven transmembrane domains which is a characteristic structural feature of GPCRs. HGPRBMY8 belongs to the “class A” of GPCR superfamily and is closely related to adrenergic and serotonin receptors based on sequence similarity. Class A is the largest sub-family of the GPCR superfamily. This particular orphan GPCR is expressed highly in brain.

HGPRBMY8 polypeptides and polynucleotides are useful for diagnosing diseases related to over- or under- expression of HGPRBMY8 proteins by identifying mutations in the HGPRBMY8 gene using HGPRBMY8 probes, or by determining HGPRBMY8 protein or mRNA expression levels. HGPRBMY8 polypeptides are also useful for screening compounds, which affect activity or function of the protein. The invention encompasses the polynucleotide encoding the HGPRBMY8 polypeptide and the use of the HGPRBMY8 polynucleotide or polypeptide, or composition thereof, in the screening, diagnosis, treatment, or prevention of disorders associated with aberrant or uncontrolled cellular growth and/or function, such as neoplastic diseases (e.g., cancers and tumors), with particular regard to diseases or disorders related to the brain, e.g. neurological disorders.

Nucleic acids encoding human HGPRBMY8 according to the present invention were first identified from the human genomic data available from GenBank (Accession No: AC016468).

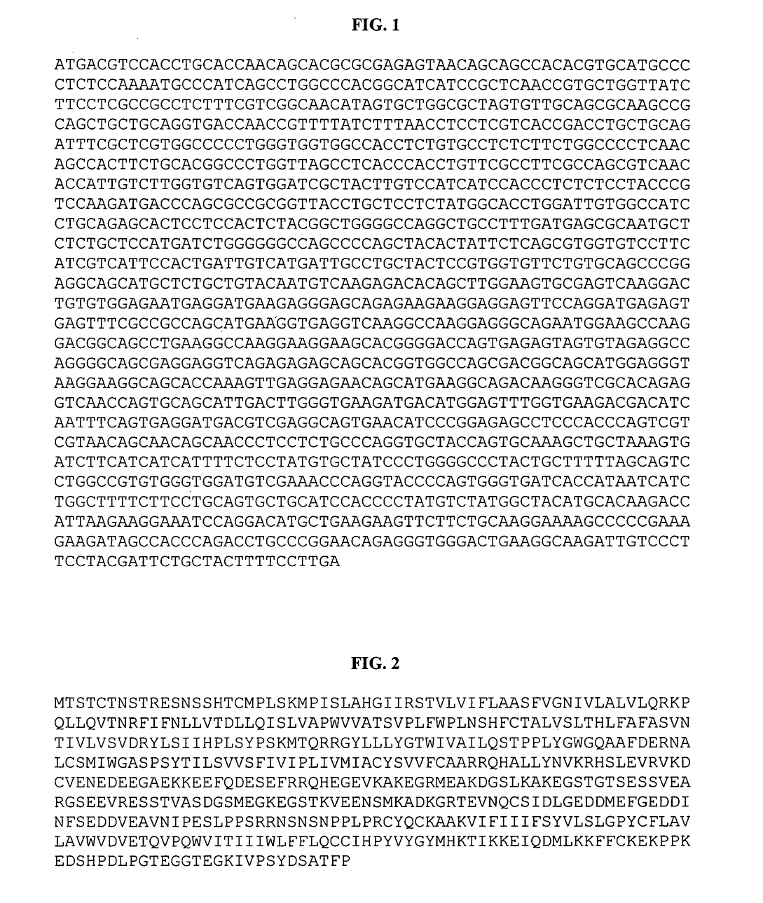

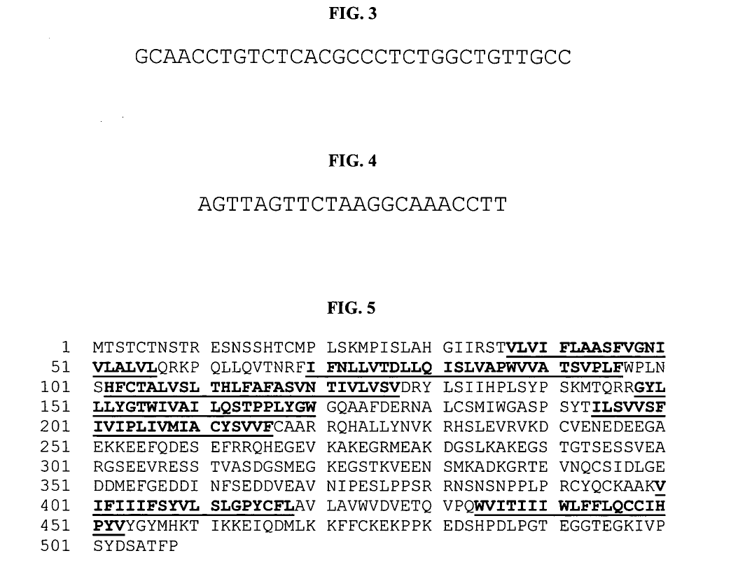

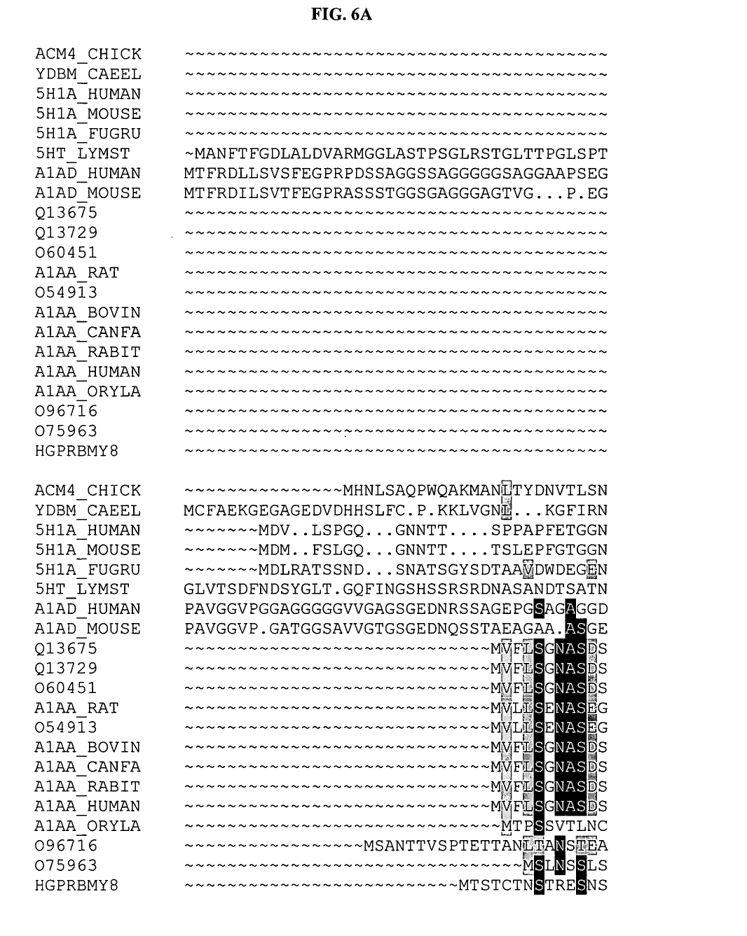

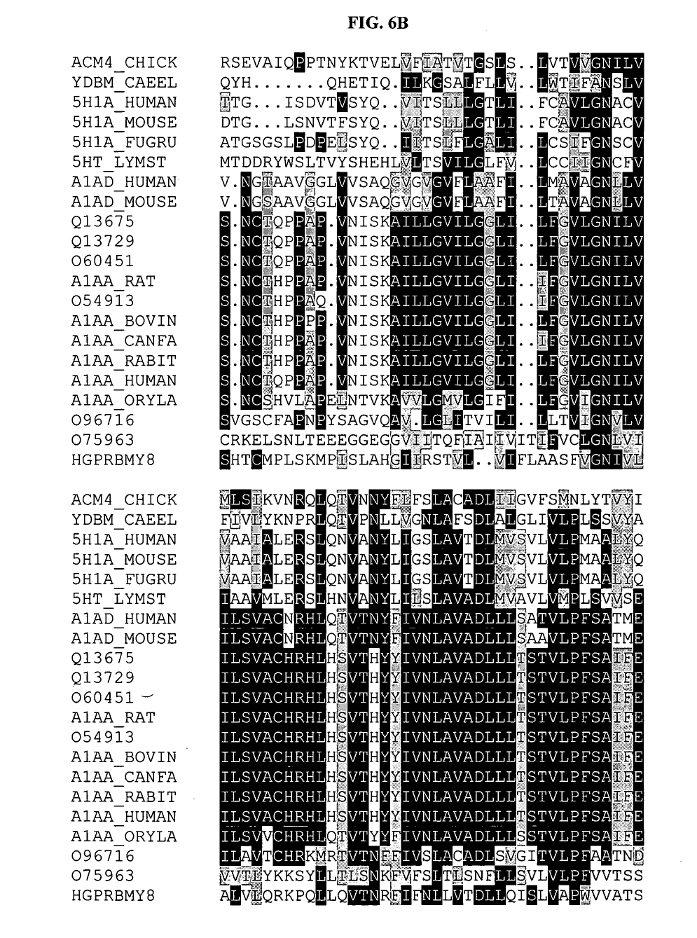

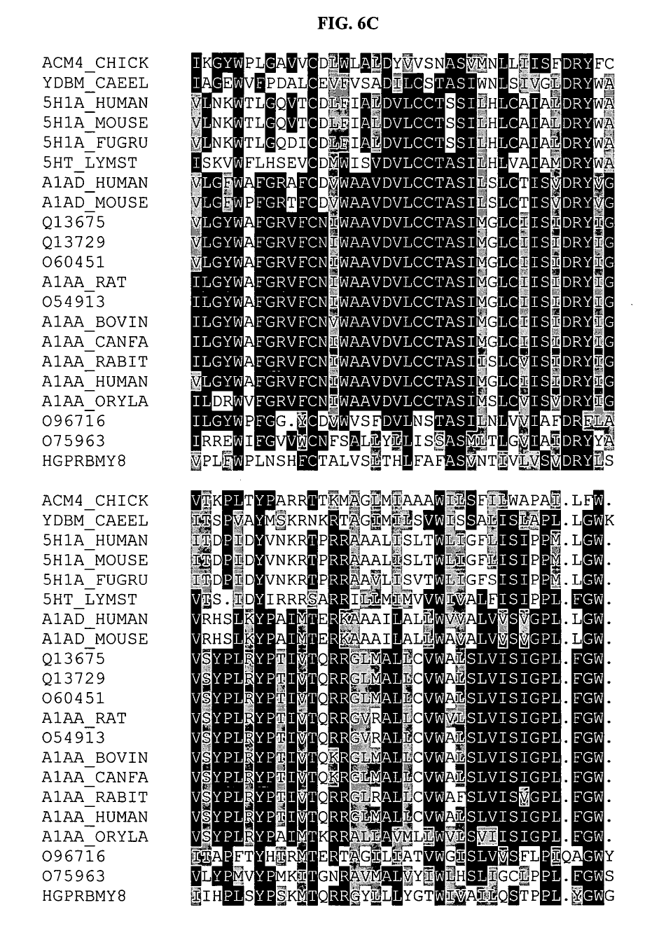

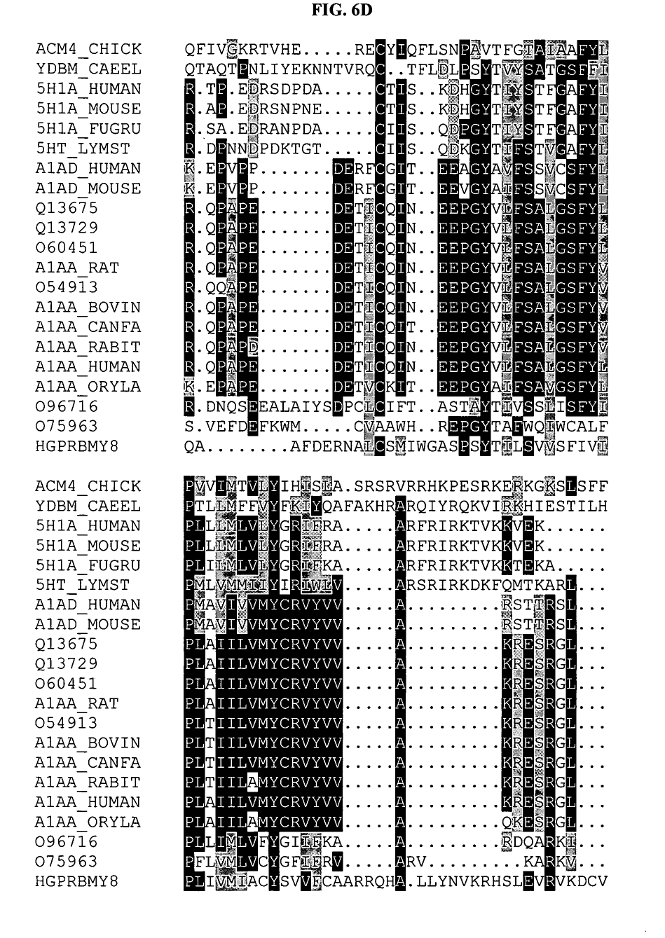

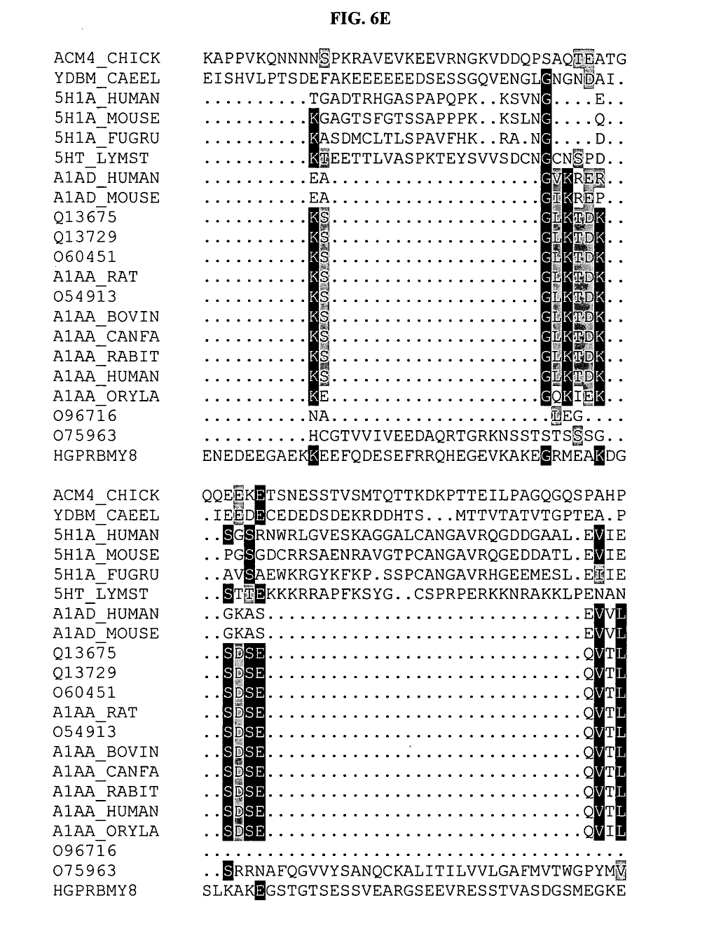

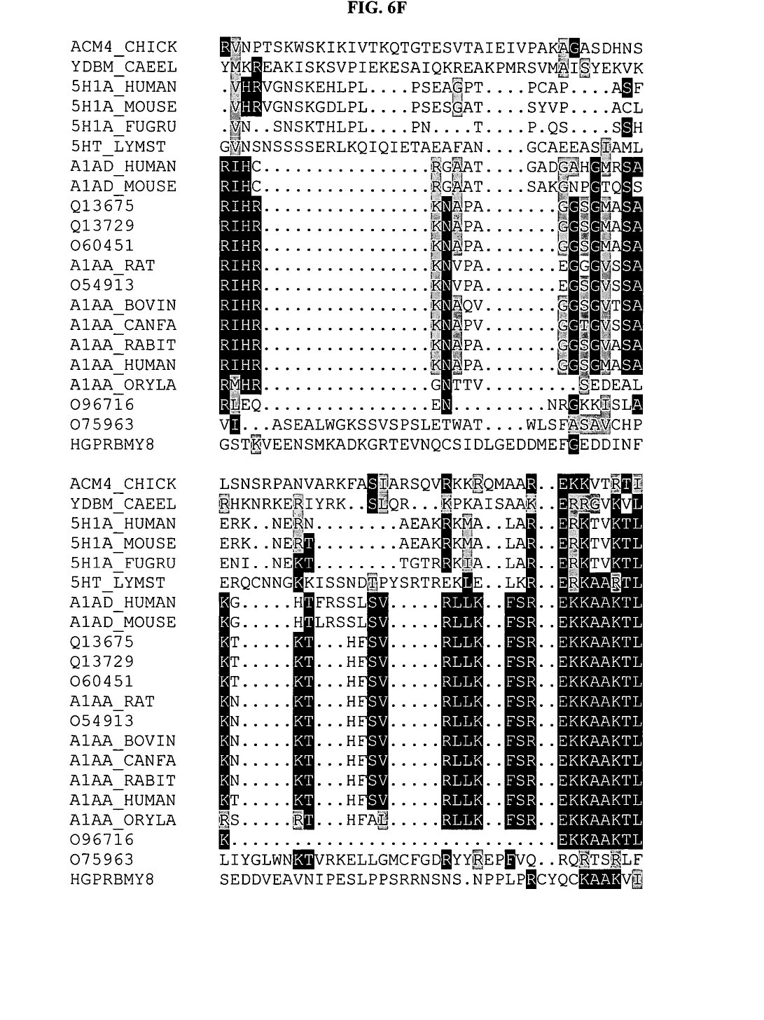

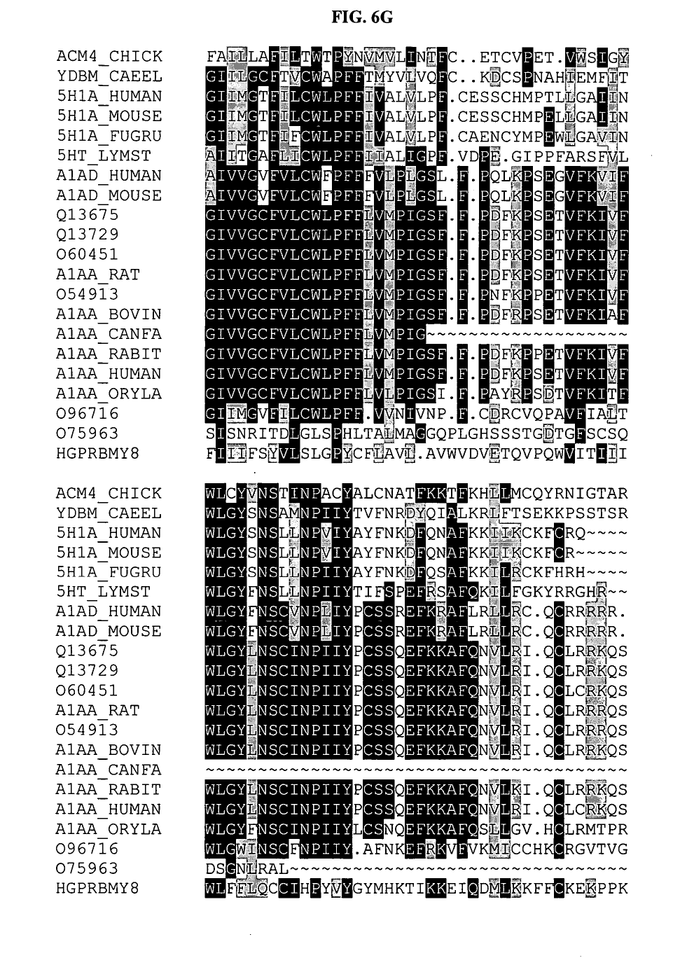

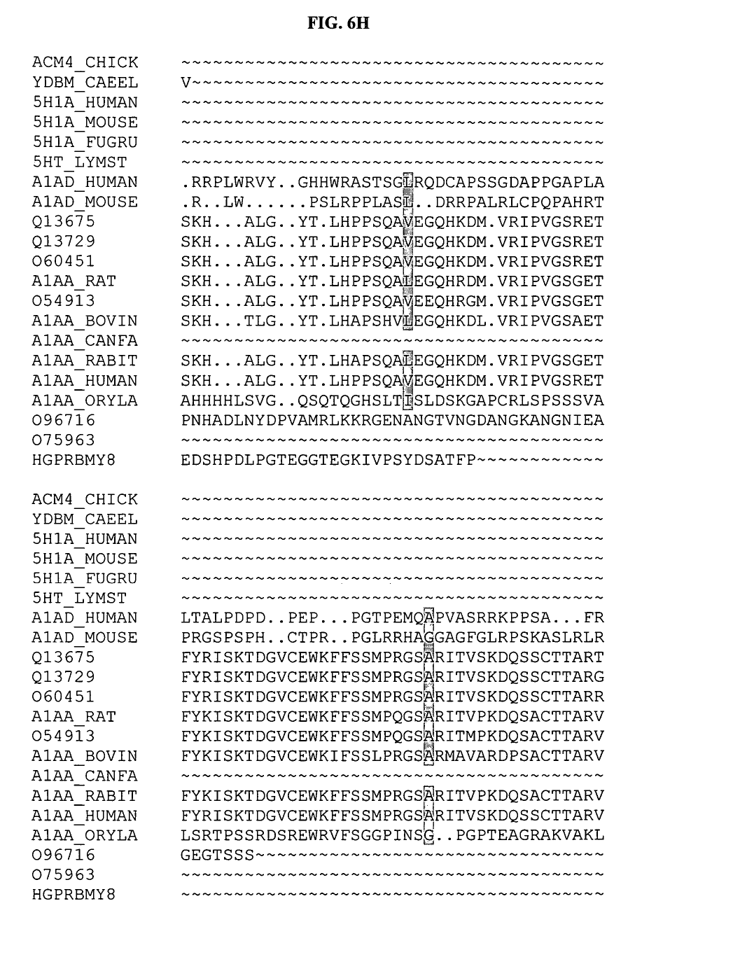

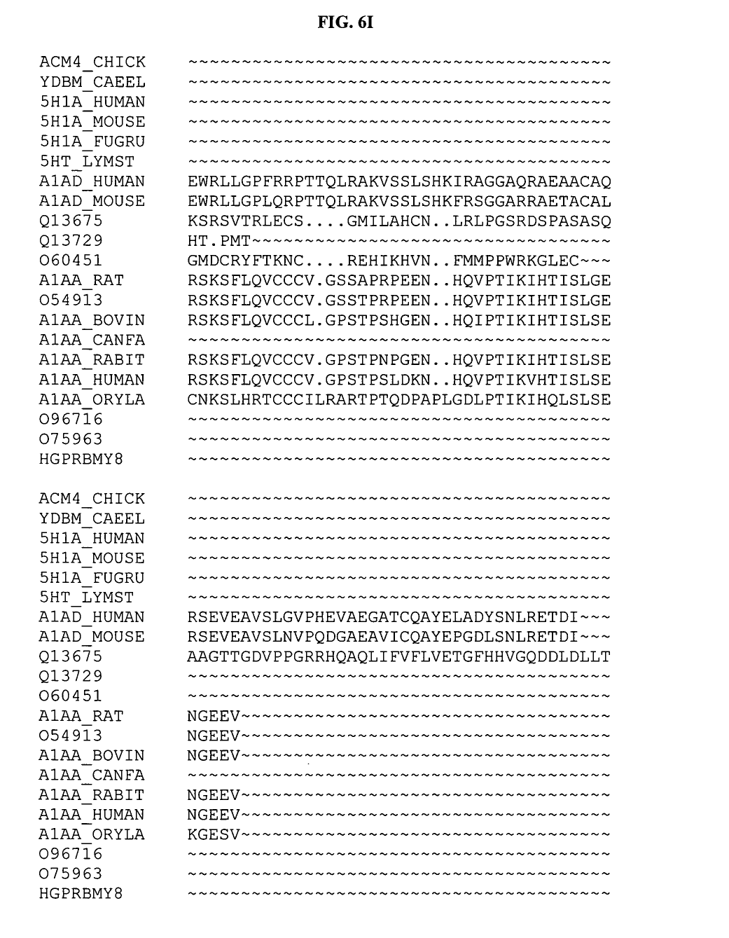

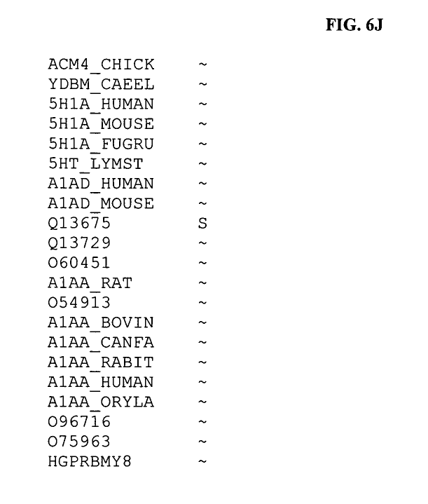

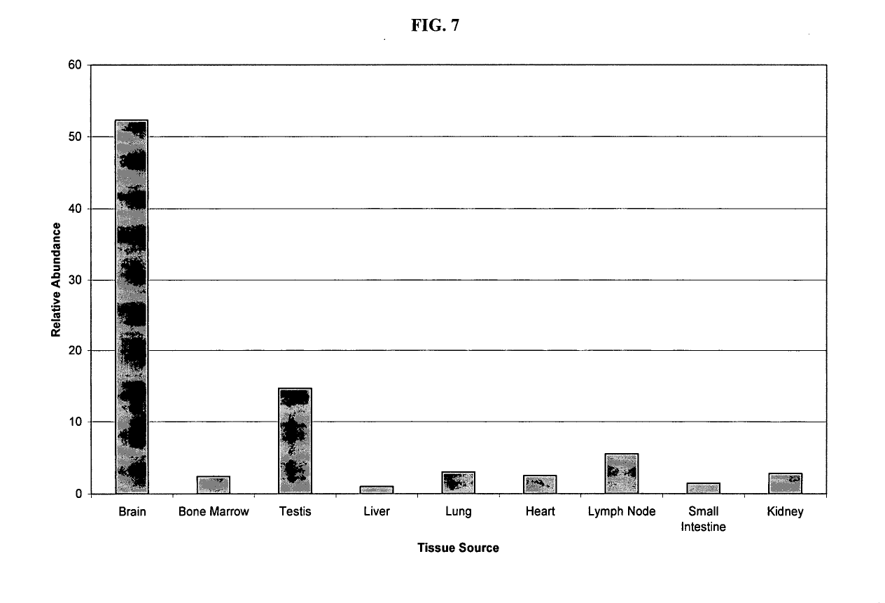

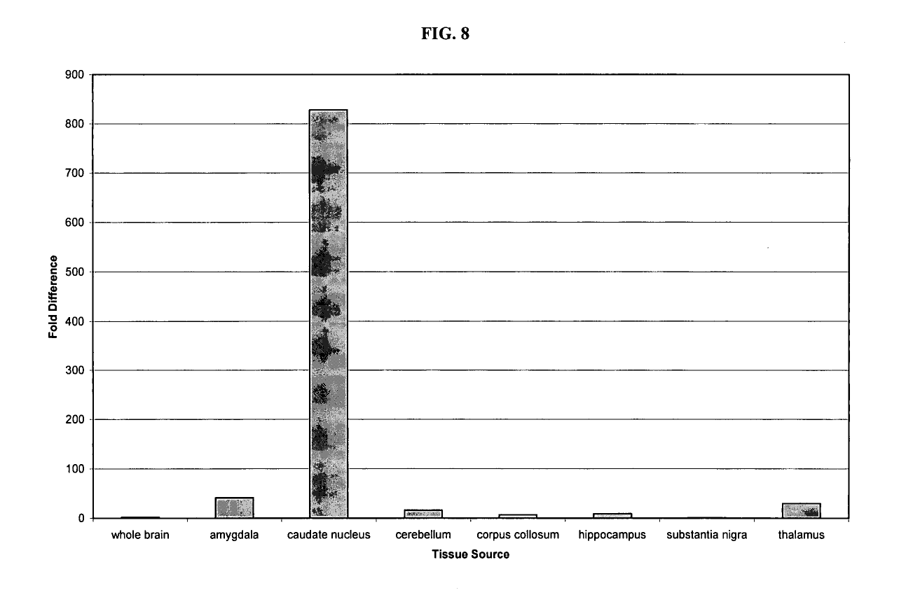

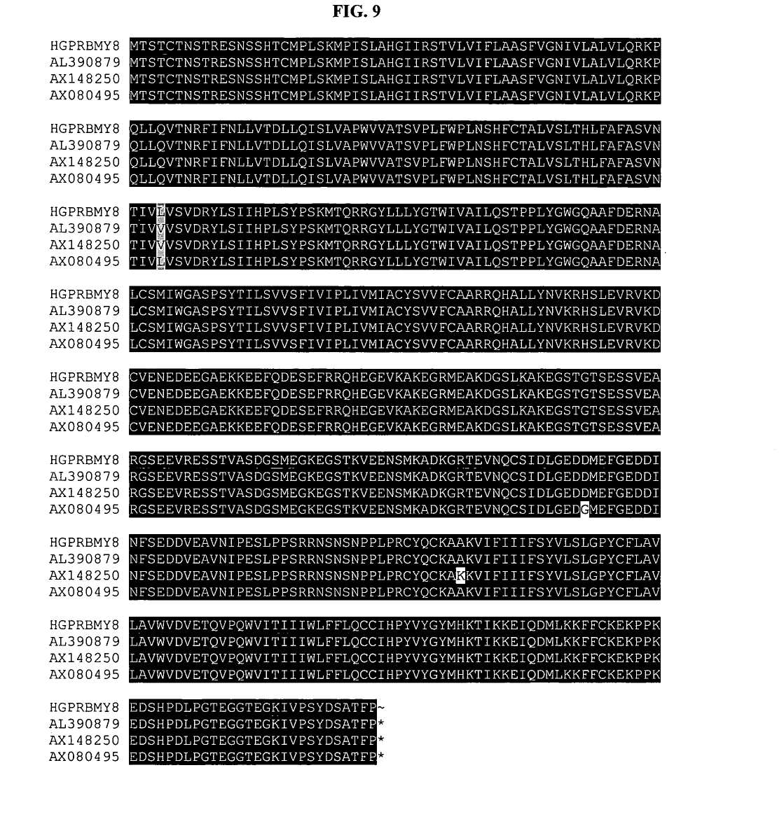

In one of its embodiments, the present invention encompasses a polypeptide comprising the amino acid sequence of SEQ ID NO:2 as shown in FIG. 1. The HGPRBMY8 polypeptide is 508 amino acids in length and shares amino acid sequence homology with the GPCR RE2. The HGPRBMY8 polypeptide (SEQ ID NO:2) shares 24.3% identity and 33.6% similarity with over 400 amino acids of the GPCR RE2 sequence, wherein “similar” amino acids are those which have the same/ similar physical properties and in many cases, the function is conserved with similar residues. For example, amino acids Lysine and Arginine are similar; while residues such as Proline and Cysteine, which do not share any physical properties, are considered dissimilar. The HGPRBMY8 polypeptide shares 28.01% identity and 38.33% similarity with the Fugu rubripes 5-Hydroxytryptamine 1a-Alpha Receptor (SHIA_FUGRU; SWISS-PROT Acc. No.:042385); 25.3% identity and 37.23% similarity with the human 5-Hydroxytryptamine 1a-Alpha Receptor (5HlA_HUMAN; SWISS-PROT Acc. No.:P08908); 27.56% idenity and 37.56% similarity with the Mus musculus 5-Hydroxytryptamine 1a-Alpha Receptor (SHIA_MOUSE; SWISS-PROT Acc. No.:Q64264, Q60956); 25.46% identity and 37.05% similarity with the Lymnaea stagnalis 5-hydroxytryptamine receptor (5HT_LYMST; SWISS-PROT Acc. No.:Q25414); 23.67% identity and 33.19% similarity with the Bos taurus Alpha-1A adrenergic receptor (A1AA_BOVIN; SWISS-PROT Acc. No.: P18130); 26.21% identity and 36.9% similarity with the Canis familiaris Alpha-1A adrenergic receptor (A1AA_CANFA; SWISS-PROT Acc. No.: 077621); 29.47% identity and 41.05% similarity with the human Alpha-lA adrenergic receptor (A1AA_HUMAN; SWISS-PROT Acc. No.: P35348); 31.65% identity and 42.29% similarity with the Oryzias latipes Alpha-1A adrenergic receptor (A1AA_ORYLA; SWISS-PROT Acc. No.:Q91175); 30% identity and 41.32% similarity with the Oryctolagus cuniculus Alpha-1A adrenergic receptor (A1AA_RABIT; SWISS-PROT Acc. No.: 002824); 24.82% identity and 34.43% similarity with the Rattus norvegicus Alpha-1A adrenergic receptor (A1AA_RAT; SWISS-PROT Acc. No.:P43140); 29.79% identity and 41.19% similarity with the human Alpha-1D adrenergic receptor (A1AD_HUMAN; SWISS-PROT Acc. No.: P25100); 29.2% identity and 40.57% similarity with the Mus musculus Alpha-1D adrenergic receptor (A1AD_MOUSE; SWISS-PROT Acc. No.:P97714, Q61619); 23.33% identity and 31.97% similarity with the Gallus gallus muscarinic acetylcholine receptor M4 (ACM4_CHICK; SWISS-PROT Acc. No.:P17200); 30.53% identity and 41.58% similarity with the Mus musculus Alpha-1A adrenergic receptor (054913; SWISS-PROT Acc. No.:054913); 29.47% identity and 41.05% similarity with the human Alpha-1A adrenergic receptor isoform 4 (060451; SWISS—PROT Acc. No.:060451); 23.59% identity and 32.82% similarity with the human G-protein coupled receptor RE2 (075963; SWISS-PROT Acc. No.:075963); 23.99% identity and 31.81% similarity with the Branchiostoma lanceolatum dopamine D1/Beta receptor (096716; SWISS-PROT Acc. No.:096716); 29.21% identity and 40.79% similarity with the human Alpha 1C adrenergic receptor isoform 2 (Q13675; SWISS-PROT Acc. No.:Q13675); 24.87% identity and 34.52% similarity with the human Alpha 1C adrenergic receptor isoform 3 (Q13729; SWISS-PROT Acc. No.:Q13729); and 21.49% identity and 32.023% similarity with the Caenorhabditis elegans probable G protein coupled receptor F01E11.5 (YDBM_CAEEL; SWISS—PROT Acc. No.:Q19084).

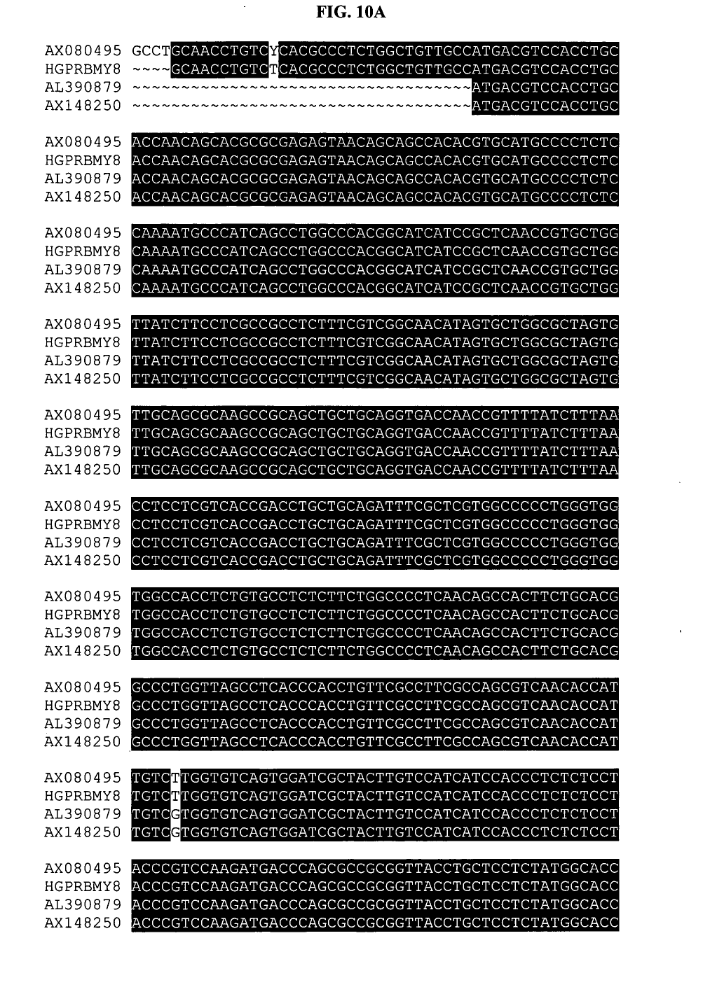

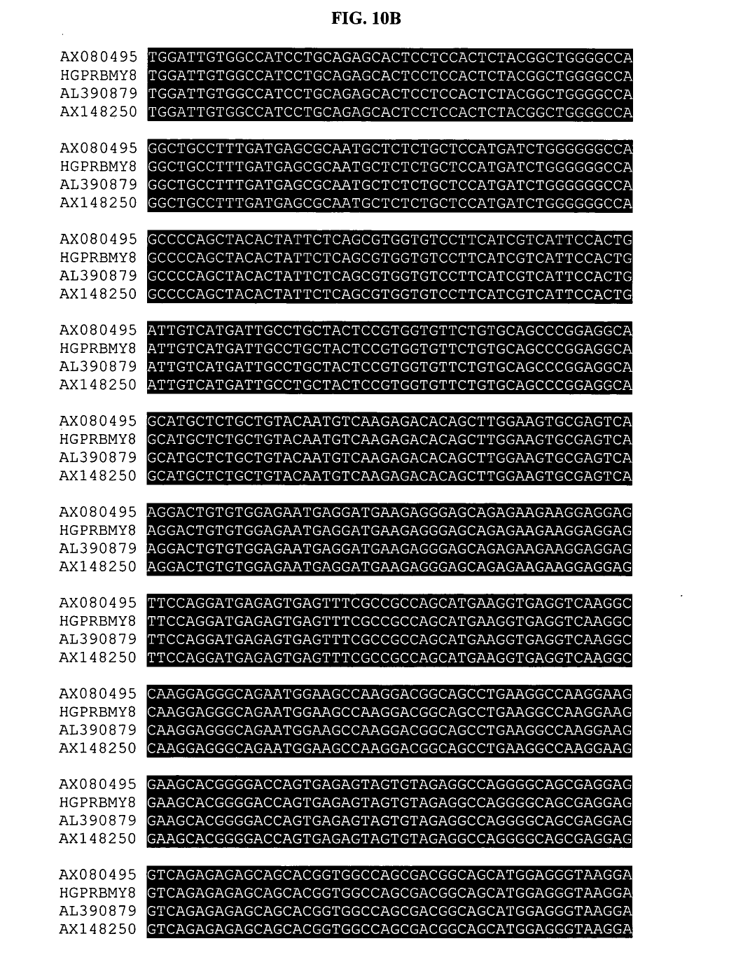

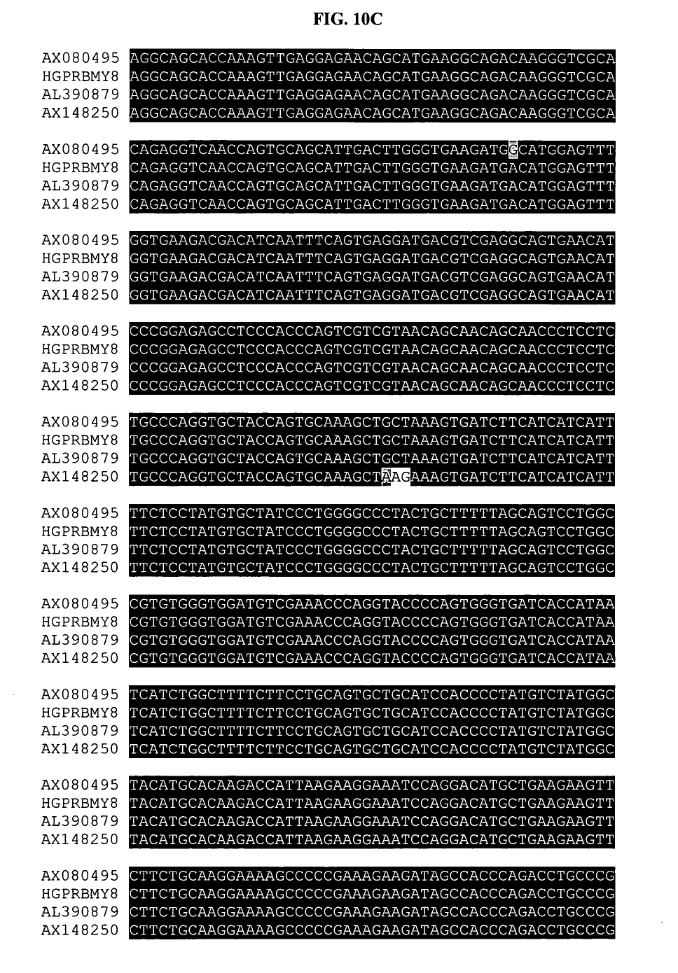

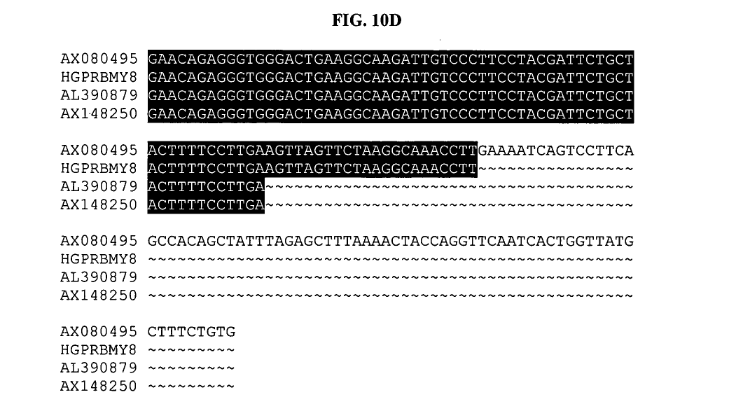

Variants of the HGPRBMY8 polypeptide are also encompassed by the present invention. A preferred HGPRBMY8 variant has at least 75 to 80%, more preferably at least 85 to 90%, and even more preferably at least 90% amino acid sequence identity to the amino acid sequence claimed herein, and which retains at least one biological, immunological, or other functional characteristic or activity of the HGPRBMY8 polypeptide. Most preferred is a variant having at least 95% amino acid sequence identity to that of SEQ ID NO:2. For example, FIGS. 9 and 10 show multiple sequence alignments of HGPRBMY8 and single nucleotide polymorphism (SNP) variants. Highlighted are the differences in sequence.

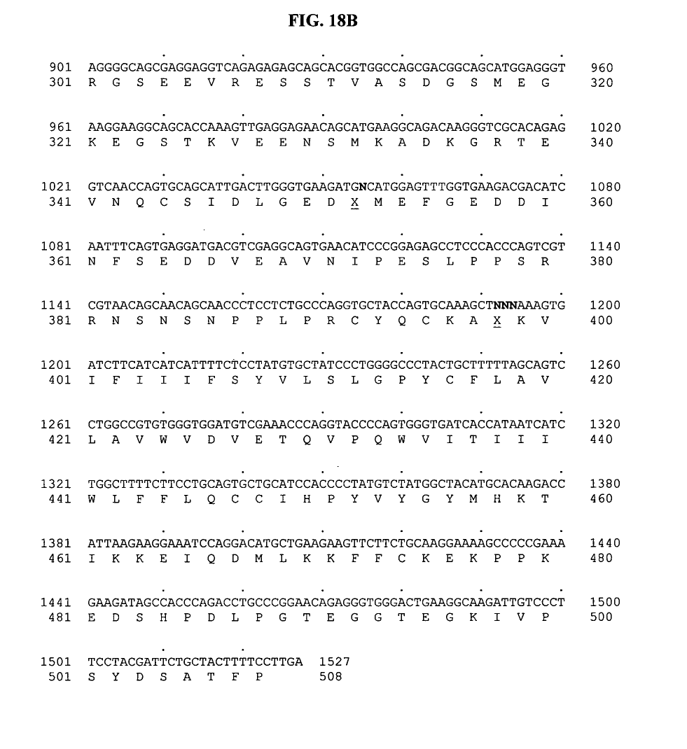

In a preferred embodiment, polynucleotide and polypeptide polymorphisms are shown in FIGS. 18A-B. The standard one-letter abbreviation for amino acids is used to illustrate the deduced amino acid sequence. The polynucleotide sequence contains a sequence of 1527 nucleotides (SEQ ID NO:48), encoding a polypeptide of 508 amino acids (SEQ ID NO:49). The polynucleotide polymorphic sites are represented by an “N”, in bold. The polypeptide polymorphic sites are represented by an “X”, and underlined. The present invention encompasses the polynucleotide at nucleotide position 370 as being either a “T” or a “G”, the polynucleotide at nucleotide position 1055 as being either a “A” or a “G”, the polynucleotide at nucleotide position 1192 as being either a “G” or a “A”, the polynucleotide at nucleotide position 1193 as being either a “C” or a “A”, and the polynucleotide at nucleotide position 1194 as being either a “T” or a “G” of FIGS. 18A-B (SEQ ID NO:48), in addition to any combination thereof. The present invention also encompasses the polypeptide at amino acid position 124 as being either a “Leu” or a “Val”, the polypeptide at amino acid position 352 as being either a “Asp” or a “Gly”, and the polypeptide at amino acid position 398 as being either a “Ala” or an “Lys” of FIGS. 18A-B (SEQ ID NO:49).

These polymorphisms are useful as genetic markers for any study that attempts to look for linkage between HGPRBMY8 and a disease or disease state related to this polypeptide.

In preferred embodiments, the following single nucleotide polymorphism polynucleotides are encompassed by the present invention:

| | |
|---|---|
| CACCATTGTCTTGGTGTCAGT, | (SEQ ID NO:50) |
| CACCATTGTCGTGGTGTCAGT, | (SEQ ID NO:51) |
| GGTGAAGATGACATGGAGTTT, | (SEQ ID NO:52) |
| GGTGAAGATGGCATGGAGTTT, | (SEQ ID NO:53) |
| GTGCAAAGCTGCTAAAGTGAT, | (SEQ ID NO:54) |
| GTGCAAAGCTACTAAAGTGAT, | (SEQ ID NO:55) |
| TGCAAAGCTGCTAAAGTGATC, | (SEQ ID NO:56) |
| TGCAAAGCTGATAAAGTGATC | (SEQ ID NO:57) |
| GCAAAGCTGCTAAAGTGATCT, | (SEQ ID NO:58) |
| and/or | |
| GCAAAGCTGCGAAAGTGATCT. | (SEQ ID NO:59) |

Polypeptides encoded by these polynucleotides are also provided.

The predicted ‘T’ to ‘G’ polynucleotide polymorphism located at nucleic acid 370 of SEQ ID NO:1 is a missense mutation resulting in a change in an encoding amino acid from ‘L’ to ‘V’ at amino acid position 124 of SEQ ID NO:2.

The predicted ‘A’ to ‘G’ polynucleotide polymorphism located at nucleic acid 1055 of SEQ ID NO:1 is a missense mutation resulting in a change in an encoding amino acid from ‘D’ to ‘G’ at amino acid position 352 of SEQ ID NO:2.

The predicted ‘G’ to ‘A’ polynucleotide polymorphism located at nucleic acid 1192 of SEQ ID NO:1 is a missense mutation resulting in a change in an encoding amino acid from ‘A’ to ‘T’ at amino acid position 398 of SEQ ID NO:2.

The predicted ‘C’ to ‘A’ polynucleotide polymorphism located at nucleic acid 1193 of SEQ ID NO:1 is a missense mutation resulting in a change in an encoding amino acid from ‘A’ to ‘D’ at amino acid position 398 of SEQ ID NO:2.

The predicted ‘T’ to ‘G’ polynucleotide polymorphism located at nucleic acid 1194 of SEQ ID NO:1 is a silent mutation and does not result in a change in amino acid.

However, taken together the predicted ‘G’ to ‘A’ polynucleotide polymorphism located at nucleic acid 1192, the predicted ‘C’ to ‘A’ polynucleotide polymorphism located at nucleic acid 1193, and the predicted ‘T’ to ‘G’ polynucleotide polymorphism located at nucleic acid 1194 of SEQ ID NO:1 represent a missense mutations resulting in a change in an encoding amino acid from ‘A’ to ‘K’ at amino acid position 398 of SEQ ID NO:2.

The present invention relates to isolated nucleic acid molecules comprising, or alternatively, consisting of all or a portion of the variant allele of the human HGPRBMY8 G-protein coupled receptor gene (e.g., wherein reference or wildtype human HGPRBMY8 G-protein coupled receptor gene is exemplified by SEQ ID NO:1). Preferred portions are at least 10, preferably at least 20, preferably at least 40, preferably at least 100, contiguous polynucleotides comprising anyone of the human HGPRBMY8 G-protein coupled receptor gene alleles described herein and exemplified in FIGS. 10A-D.

In one embodiment, the invention relates to a method for predicting the likelihood that an individual will have a disorder associated with the reference allele at nucleotide position 370, 1055, 1192, 1193, and/or 1194 of SEQ ID NO:1 (or diagnosing or aiding in the diagnosis of such a disorder) comprising the steps of obtaining a DNA sample from an individual to be assessed and determining the nucleotide present at position 370, 1055, 1192, 1193, and/or 1194 of SEQ ID NO:1. The presence of the variant allele at this position indicates that the individual has a greater likelihood of having a disorder associated therewith than an individual having the reference allele at that position, or a greater likelihood of having more severe symptoms.

Conversely, the invention relates to a method for predicting the likelihood that an individual will have a disorder associated with the variant allele at nucleotide position 370, 1055, 1192, 1193, and/or 1194 of SEQ ID NO:1 (or diagnosing or aiding in the diagnosis of such a disorder) comprising the steps of obtaining a DNA sample from an individual to be assessed and determining the nucleotide present at position 370, 1055, 1192, 1193, and/or 1194 of SEQ ID NO:1. The presence of the variant allele at this position indicates that the individual has a greater likelihood of having a disorder associated therewith than an individual having the reference allele at that position, or a greater likelihood of having more severe symptoms.

The present invention further relates to isolated proteins or polypeptides comprising, or alternatively, consisting of all or a portion of the encoded variant amino acid sequence of the human HGPRBMY8 G-protein coupled receptor polypeptide (e.g., wherein reference or wildtype human HGPRBMY8 G-protein coupled receptor polypeptide is exemplified by SEQ ID NO:2). Preferred portions are at least 10, preferably at least 20, preferably at least 40, preferably at least 100, contiguous polypeptides and comprises any one of the amino acid variant alleles of the human HGPRBMY8 G-protein coupled receptor polypeptide exemplified in FIGS. 18A-B, or a portion of SEQ ID NO:49. Alternatively, preferred portions are at least 10, preferably at least 20, preferably at least 40, preferably at least 100, contiguous polypeptides and comprises any one of the amino acid reference alleles of the human HGPRBMY8 G-protein coupled receptor protein exemplified in FIGS. 18A-B, or a portion of SEQ ID NO:49. The invention further relates to isolated nucleic acid molecules encoding such polypeptides or proteins, as well as to antibodies that bind to such proteins or polypeptides.

In another embodiment, the present invention encompasses polynucleotides, which encode the HGPRBMY8 polypeptide. Accordingly, any nucleic acid sequence, which encodes the amino acid sequence of HGPRBMY8 polypeptide, can be used to produce recombinant molecules that express HGPRBMY8 protein. In a particular embodiment, the present invention encompasses the HGPRBMY8 polynucleotide comprising the nucleic acid sequence of SEQ ID NO:1 as shown in FIG. 1. More particularly, the present invention provides the HGPRBMY8 clone. More particularly, the present invention provides the HGPRBMY8 clone, deposited at the American Type Culture Collection (ATCC), 10801 University Boulevard, Manassas, Va. 20110-2209 on Jan. 24, 2001 and under ATCC Accession No. PTA-2966 according to the terms of the Budapest Treaty.

As will be appreciated by the skilled practitioner in the art, the degeneracy of the genetic code results in the production of a number of nucleotide sequences encoding HGPRBMY8 polypeptide. Some of the sequences bear minimal homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring HGPRBMY8, and all such variations are to be considered as being specifically disclosed.

Although nucleotide sequences which encode HGPRBMY8 polypeptide and its variants are preferably capable of hybridizing to the nucleotide sequence of the naturally occurring HGPRBMY8 polypeptide under appropriately selected conditions of stringency, it may be advantageous to produce nucleotide sequences encoding HGPRBMY8 polypeptide, or its derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Other reasons for substantially altering the nucleotide sequence encoding HGPRBMY8 polypeptide, and its derivatives, without altering the encoded amino acid sequences include the production of RNA transcripts having more desirable properties, such as a greater half-life, than transcripts produced from the naturally occurring sequence.

The present invention also encompasses production of DNA sequences, or portions thereof, which encode the HGPRBMY8 polypeptide, and its derivatives, entirely by synthetic chemistry. After production, the synthetic sequence may be inserted into any of the many available expression vectors and cell systems using reagents that are well known and practiced by those in the art. Moreover, synthetic chemistry and other known techniques may be used to introduce mutations into a sequence encoding HGPRBMY8 polypeptide, or any fragment thereof.

In preferred embodiments, the present invention encompasses a polynucleotide lacking the initiating start codon, in addition to, the resulting encoded polypeptide of HGPRBMY8. Specifically, the present invention encompasses the polynucleotide corresponding to nucleotides 4 thru 1524 of SEQ ID NO:1, and the polypeptide corresponding to amino acids 2 thru 508 of SEQ ID NO:2. Also encompassed are recombinant vectors comprising said encoding sequence, and host cells comprising said vector.

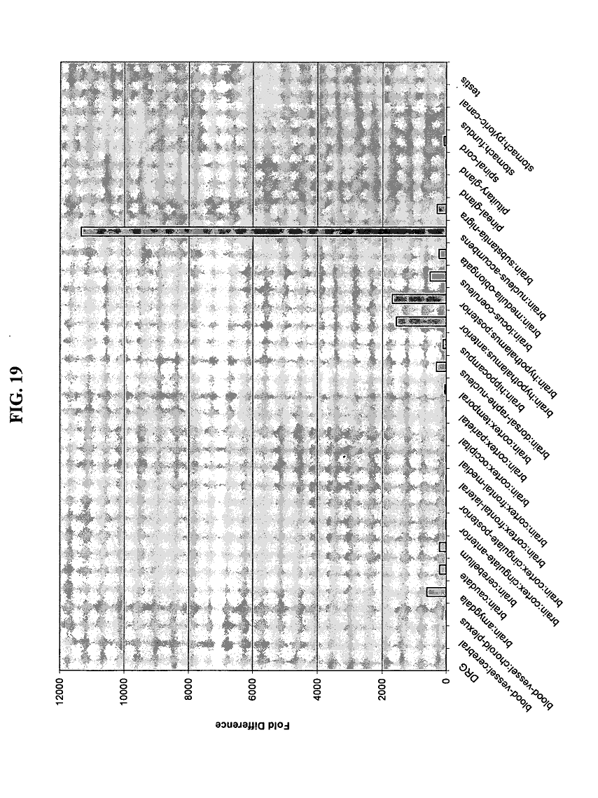

Expanded analysis of HGPRBMY8 expression levels by TaqMan™ quantitative PCR (see FIG. 19) confirmed that the HGPRBMY8 polypeptide is expressed in the brain (FIG. 7), and in particular confirmed the expression in several brain-subregions (FIG. 8). HGPRBMY8 was expressed predominately in the nervous system, with predominate expression in the nucleus accumbens. Expression of HGPRBMY8 was also significantly expressed in other brain sub-regions, including the hypothalamus, amygdala, and to a lesser extent in the hippocampus, caudate, cerebellum, raphe nucleus, the locus coeruleus, and the medulla oblongata.

Collectively the expression data suggests HGRPBMY8 may play a role in neural processes that connect the nucleus accumbens to its ‘reward center’ functions which include the release of neurotransmitters such as dopamine, opioid peptides, serotonin, and GABA.

The HGPRBMY8 polynucleotides and polypeptides, including fragments, variants, and modulators thereof, may be useful in treating, diagnosing, prognosing, and/or preventing neurological disease states, including nucleus accumbens disorders, caudate nucleus disorders, neurotransmitter disorders, neurotransmitter expression disorders, neurotransmitter release disorders, disorders associated with aberrant dopamine synthesis, disorders associated with aberrant dopamine release, disorders associated with aberrant dopamine function, disorders associated with aberrant opioid peptide synthesis, disorders associated with aberrant opioid peptide release, disorders associated with aberrant opioid peptide function, disorders associated with aberrant serotonin synthesis, disorders associated with aberrant serotonin release, disorders associated with aberrant serotonin function, disorders associated with aberrant GABA synthesis, disorders associated with aberrant GABA release, disorders associated with aberrant GABA function, affective disorders, depression, aggressive behavioral disorders, addictive disorders, etc.

The expression data also suggests that modulators of HGPRBMY8 function may have utility in treating, diagnosing, prognosing, and/or preventing neuro- pathologies, including responses to stress, and propensity to develop addictive behaviors, as well as a neuroendocrine abnormalities including anxiety, sleep and eating disorders.

Comparison of the expanded expression profile of HGPRBMY8 to the expression profile of another G-protein coupled receptor, HGPRBMY34, resulted in the appreciation that the expression profile of HGPRBMY8 is identical to the profile of HGPRBMY34 using a nearest neighbor analysis. The comparison of both expression profiles is statistically significant with a Z score of-6.9. This result strongly indicates that the expression of both HGPRBMY8 and HGPRBMY34 are co-regulated. Such co-regulation is likely physiologically relevant. The data also suggests that HGPRBMY8 and HGPRBMY34 may be capable of forming heterodimers with each other resulting in a unique complex that has physiological relevance. Such heterodimer formation may ultimately be involved in the formation and release of GABA from L-glutamic acid. Alterations in this signaling pathway might contribute substantially to the development of numerous pathophysiology conditions such as schizophrenia, Parkinson's disease, progressive supranuclear palsy, and Alzheimer's.

The formation of G-protein coupled receptor heterodimers is well known in the art, and is often inferred to occur with G-protein coupled receptors that have shared expression patterns. Such heterodimers are physiologically relevant and result in altered receptor function (Nature, 399(6737):697-700 (1999)).

For example, the first heterodimeric G-protein coupled receptor identified was the GABA(B) receptor (Bowery and Enna, J. Pharmacol Esxp Ther, 292:2-7, (2000)). The GABA(B) receptor is a heterodimer of both GABA(B1)R and GABA(B2)R receptors. According to Li et al (Mol Cells. 16(1):40-7 (2003)) the GABA(B1)R subunit is essential for ligand binding while the GABA(B2)R subunit is essential for cell surface localization. The expression pattern of both receptors is highly parallel. The activity of GABA(B) absolutely requires the formation of the GABA(B1)R and GABA(B2)R heterodimer as homodimers of either GPCR does not result in a functional GABA(B) receptor. See also Sands et al (J Pharmacol Exp Ther. 305(1):191-6 (2003)).

Another important group of G-protein coupled receptors localized in the brain that are known to form heterodimers are the Neurotensin receptor-1 and -3 receptors. Both receptors share the same expression profile and were demonstrated to form heterodimers by immunoprecipitation (see Martin et al Gastroenterology. 123(4):1135-43 (2002)).

The Oxytocin and Vasopressin V1a and V2 receptors are another group of G-protein coupled receptors that are known to form heterodimers based upon coimmunoprecipitation and bioluminescence resonance energy transfer experiments (see Terrillon et al Mol Endocrinol. 17(4):677-91 (2003)).

In fact, studies involving the heterodimerization of sst(2A) and sst(3) somatostatin receptors have demonstrated that such heterodimers result in inactivation of sst(3) receptor function (Pfeiffer, M., Koch, T., Schroder, H., Klutzny, M., Kirscht, S., Kreienkamp, H. J., Hollt, V., and Schulz, S. (2001) J. Biol. Chem. 276, 14027-14036)).

Similar studies investigating the affect of heterodimerization of sst(2A) and MOR1 in human embryonic kidney 293 cells determined that sst(2A)-MOR1 heterodimerization did not substantially alter the ligand binding or coupling properties of these receptors, however, exposure of the sst(2A)-MOR1 heterodimer to the sst(2A)-selective ligand L-779,976 induced phosphorylation, internalization, and desensitization of sst(2A) as well as MOR1. Similarly, exposure of the sst(2A)-MOR1 heterodimer to the mu-selective ligand [d-Ala(2),Me-Phe(4),Gly(5)-ol]enkephalin induced phosphorylation and desensitization of both MOR1 and sst(2A) but not internalization of sst(2A). Thus, it was concluded that heterodimerization may represent a novel regulatory mechanism that could either restrict or enhance phosphorylation and desensitization of G protein-coupled receptors (J Biol Chem., 277(22):19762-72 (2002)).

The HGPRBMY8 polynucleotides and polypeptides, including fragments, variants, and modulators thereof, may be useful in treating, diagnosing, prognosing, and/or preventing disorders associated with the release of GABA from L-glutamic acid, schizophrenia, Parkinson's disease, progressive supranuclear palsy, and Alzheimer's, affective disorders, depression, aggressive behavioral disorders, and addictive disorders.

The present invention encompasses host cells comprising one or more recombinant vectors that contain either the entire or partial coding region of HGPRBMY8 and HGPRBMY34. Such recombinant vectors may contain the coding region of either HGPRBMY8 or HGPRBMY34 individually (e.g., one coding region per vector) in which case co-expression of HGPRBMY8 and HGPRBMY34 would require co-transfection. Alternative, such recombinant vectors may contain both HGPRBMY8 and HGPRBMY34 coding regions on the same vector, in which case only single transfection would be required.

Cell lines co-expression both HGPRBMY8 and HGPRBMY34 would be useful in screens designed to identify modulators of either HGPRBMY8 or HGPRBMY34, individually, or modulators that are capable of interefering with the ability of HGPRBMY8 and HGPRBMY34 to form heterodimers. Such modulators may be small molecules, antibodies, antisense reagents, peptides, etc.

Additional comparison of the expanded expression profile of HGPRBMY8 to the expression profile of glutamate carboxylase (SEQ ID NO:104; Genbank Accession No. NM_{—}000818) using a nearest neighbor analysis, resulted in the appreciation that the expression profile of HGPRBMY8 is very similar to the profile of glutamate carboxylase. The comparison of both expression profiles is statistically significant with a Z score of −6.1. This result strongly indicates that the expression of both HGPRBMY8 and glutamate carboxylase are co-regulated. Such co-regulation is likely physiologically relevant.

The shared expression pattern of HGPRBMY8 to glutamate carboxylase is significant since glutamate carboxylase is a rate limiting enzyme involved in the synthesis of GABA. Based upon the its shared expression pattern, once skilled in the art would reasonable believe that HGPRBMY8 is involved in the regulation, either directly, or indirectly, of GABA synthesis.

In further confirmation of this expression pattern, antibodies specific to an HGPRBMY9 epitope (SEQ ID NO:114) were used to assess the expression pattern of HGPRBMY9 using IHC (see Example 16). The expression pattern using anti-HGPRBMY9 antibodies was essential the same as the expression data obtained by TaqMan analysis and SYBR green. Briefly, the IHC results showed that antibody specific to HGPRBMY9 selectively stained the neuropil of the amygdala, the amygdalotemporal cortex, the inferior-temporal cortex, the orbitofrontal cortex, the entorhinal cortex, the subiculum, areas CA1 through CA4, the hypothalamus, the substantia nigra, the hypoglossal, solitary, gracile, cuneate, lateral cuneate, trigeminal, arcuate, and olivary nuclei in the medulla, and the nucleus of Clarke in the spinal cord. Many neurons in the amygdala, the caudate, the putamen, the basal striatum, the claustrum, hippocampal areas CA1 through CA4, the cortex, the substantia innominata, the nucleus basalis of Meynert, the paraventricular, tuberal, posterior hypothalamic and posterior lateral hypothalamic nuclei, and the thalamus were stained strongly. Many neurons in the orbitofrontal cortex and the amygdalotemporal cortex showed faint to moderate staining. Stellate, basket, and Purkinje neurons in the cerebellum, the lateral geniculate body and nuclei in the medulla showed faint to moderate staining. Rarely, strong staining was found in protoplasmic astrocytes—a subset of astrocytes intimately associated with neurons—in the amygdala, hippocampus, cerebral cortex, and anterior nuclear group of the thalamus. A few myelinated nerve tracts or fibers showed faint staining in a sample of thoracic spinal cord. Oligodendrocytes and microglia were negative. The choroid plexus epithelium showed moderate to strong staining. Ependymal cells showed faint to moderate staining. In the pituitary, the pars intermedia and Herring bodies in the pars posterior stained strongly. Generally, vascular smooth muscle cells stained moderately. In conclusion, this antibody mainly stained neurons and neuropil in many brain samples.

In summary, the analysis of the mRNA and protein expression patterns for HGPRBMY8 demonstrated that this GPCR is exclusively expressed in the brain, suggesting this receptor plays a role in regulation of CNS processes. Furthermore, this GPCR exhibits an intriguing expression profile as it is expressed within several brain regions that are connected both neurochemically and functionally, and likely plays a role in processes underlying many aspects of behavior including the regulation of mood, reward, cognition, and appetite.

HGPRBMY8 expression in the nucleus accumbens (NAc) suggests this receptor is likely to play a role in the basic processes underlying reward and addictive behavior (Charney, D. S., Nestler, E. J., Bunney, B. S. Neurobiology of Mental Illness. Oxford University Press. Copyright 1999, New York, N.Y.; Barrot M, Olivier J D, Perrotti L I, DiLeone R J, Berton O, Eisch A J, Impey S, Storm D R, Neve R L, Yin J C, Zachariou V, Nestler E J, Proc Natl Acad Sci USA. 2002 Aug. 20;99(17):11435-40; and Phillips A G, Ahn S, Howland J G. Neurosci Biobehav Rev. 2003 October;27(6):543-54).

The nucleus accumbens is part of the striatum within the basal ganglia, and receives afferent connections from other brain regions (Nestler E J, Barrot M, DiLeone R J, Eisch A J, Gold S J, Monteggia L M. Neurobiology of depression. Neuron. 2002 Mar. 28;34(1):13-25; Charney, D. S., Nestler, E. J., Bunney, B. S. Neurobiology of Mental Illness. Oxford University Press. Copyright 1999, New York, N.Y.; and Martin, J. H. Neuroanatomy: Text and Atlas. Elsevier Science Publishing Co.). For example, the ventral tegmental area (VTA) sends dopaminergic projections to the NAc as part of the mesolimbic dopaminergic system. This system is important for the drug reinforcing properties of drugs of abuse, and may play a role in responses to natural reinforcers of behavior under normal conditions and compulsive responses under pathological conditions (Charney, D. S., Nestler, E. J., Bunney, B. S. Neurobiology of Mental Illness. Oxford University Press. Copyright 1999, New York, N.Y.; Barrot M, Olivier J D, Perrotti L I, DiLeone R J, Berton O, Eisch A J, Impey S, Storm D R, Neve R L, Yin J C, Zachariou V, Nestler E J, Proc Natl Acad Sci USA. 2002 Aug. 20;99(17):11435-40; and Phillips A G, Ahn S, Howland J G. Neurosci Biobehav Rev. 2003 October;27(6):543-54). Consistent with this role, disruption of G protein signaling within the nucleus accumbens attenuates the self-administration properties of cocaine and heroin, suggesting modulation of GPCR signaling within the nucleus accumbens may have utility in the treatment of addiction.

Specifically, expression of HGPRBMY8 in the amygdala may be important in processes involved in responses to conditioned fear and to rewarding stimuli. The role of the amygdala in these responses makes this brain region important in the processes underlying anxiety and depression (Nestler E J, Barrot M, DiLeone R J, Eisch A J, Gold S J, Monteggia L M. Neurobiology of depression. Neuron. 2002 Mar. 28;34(1):13-25; Charney, D. S., Nestler, E. J., Bunney, B. S. Neurobiology of Mental Illness. Oxford University Press. Copyright 1999, New York, N.Y.). The information processed by the amygdala is projected to several regions including the hypothalamus and monoaminergic nuclei, which also exhibit HGPRBMY8 expression.

The specific hypothalamic expression of HGPRBMY8 suggests this GPCR may play a role in the processes underlying regulation of the stress response as well as appetite. The hypothalamus is an important brain structure that mediates many neuroendocrine and neurovegetative functions and dysregulation of the hypothalamus and the hypothalamic-pituitary-adrenal (HPA) axis in depression is the most robust confirmed neurobiological finding among these patients (Nestler E J, Barrot M, DiLeone R J, Eisch A J, Gold S J, Monteggia L M. Neurobiology of depression. Neuron. 2002 Mar. 28;34(1):13-25; and Chamey, D. S., Nestler, E. J., Bunney, B. S. Neurobiology of Mental Illness. Oxford University Press. Copyright 1999, New York, N.Y.). It is estimated 30-40% of depressed patients exhibit dysregulation of the HPA axis. Therefore, expression of HGPRBMY8 in the hypothalamus suggests a potential role for the receptor in regulating the stress response and for the treatment of depression. In addition, many neuropeptides that modulate feeding behavior exert their effects through activity in the hypothalamus, suggesting HGPRBMY8 may either directly, or indirectly, be involved in regulating the signaling pathways involved in appetite and feeding behavior.

Collectively, HGPRBMY8 has a very intriguing expression profile, exhibiting expression in several brain regions that are known to be connected through shared function and through shared connections. For example, HGPRBMY8 exhibits its highest level of expression throughout a network of brain regions including the NAc, amygdala and hypothalamus, that are interconnected functionally to regulate processes involved in the regulation of mood, reward, and response to fearful and stressful conditions. Consistent with this proposed role, lower relative levels of HGPRBMY8 expression are detected in the raphe nucleus and the locus coeruleus, the major serotonergic and noradrenergic nuclei of the brain. These data suggest that modulators of HGPRBMY8 function may have utility in a variety of neuropathologies, including responses to stress and addictive behaviors, as well as neuroendocrine abnormalities including anxiety, depression and eating disorders.

Based upon the expression pattern of HGPRBMY8 within the brain, several neurochemical and behavioral studies are available to assess HGPRBMY8 activity in vivo. For example, neurochemical studies can be performed using microdialysis in the mesolimbic dopaminergic system to quantitate the effects of modulating HGPRBMY8 activity on dopamine release in the NAc (Robinson T. E. and Justice J. B. Jr, eds. (1991) Microdialysis in the Neurosciences, Vol. 7, Elsevier, Amsterdam; Microdialysis—theory and application. Benveniste H, Huttemeier PC. Prog Neurobiol. 1990;35(3):195-215; Parrot S, Bert L, Mouly-Badina L, Sauvinet V, Colussi-Mas J, Lambas-Senas L, Robert F, Bouilloux J P, Suaud-Chagny M F, Denoroy L, Renaud B. Cell Mol Neurobiol. 2003 October;23(4-5):793-804; which are all hereby incorporated by reference in their entirety herein). Related studies, based upon the expression of HGPRBMY8 in the raphe nucleus and locus coeruleus, could involve using microdialysis to assess the effects of modulating HGPRBMY8 activity on serotonin or norepinephrine levels (Robinson T. E. and Justice J. B. Jr, eds. (1991) Microdialysis in the Neurosciences, Vol. 7, Elsevier, Amsterdam; Microdialysis—theory and application. Benveniste H, Huttemeier P C. Prog Neurobiol. 1990;35(3):195-215; Parrot S, Bert L, Mouly-Badina L, Sauvinet V, Colussi-Mas J, Lambas-Senas L, Robert F, Bouilloux J P, Suaud-Chagny M F, Denoroy L, Renaud B. Cell Mol Neurobiol. 2003 October;23(4-5):793-804; which are all hereby incorporated by reference in their entirety herein). The role of HGPRBMY8 in the hypothalamus could also be addressed by determining the effects of HGPRBMY8 modulators on readouts of HPA axis activity, such as ACTH levels. In addition, many animal models of behavior are available for assessing the potential role of HGPRBMY8 in anxiety and depression. For example, in vivo models of anxiety and depression that exhibit predictive validity include the forced swim test, tail suspension test, elevated plus maze and situational anxiety (Cryan J F, Markou A, Lucki I.,Trends Pharmacol Sci. 2002 May;23(5):238-45; Dalvi A, Lucki I. Psychopharmacology (Berl). 1999 November;147(1):14-6; Lucki I., Behav Pharmacol. 1997 November;8(6-7):523-32; which are all hereby incorporated by reference in their entirety herein). Thus, based upon the expression pattern of HGPRBMY8 within the brain, there are several assays available to address the effects of modulating HGPRBMY8 activity in vivo on neurochemical markers as well as in behavioral outcomes.

Additionally, characterization of the HGPRBMY8 polypeptide of the present invention using antisense oligonucleotides led to the determination that HGPRBMY8 is involved in the negative modulation of the p21 G1/G2 cell cycle check point modulatory protein as described in Example 17 herein.

Antisense oligonucleotides directed against the HGPRBMY8 mRNA resulted in a marked increase in p21 expression and/or activity (see Example 17). The level of expression of p21 in response to treatment with antisense specific to HGPRBMY8 was decreased by about 43%. The results were replicated in three independent experiments and determined to be statistically significant.

These results suggest that induction of HGPRBMY8 activity or expression with a modulator would induce differentiation, and stop cellular proliferation, as p21 is a cell cycle inhibitor and is known to be associated with committment down a differentiation pathway. Numerous known drugs in clinical trials (such as, for example, cdk2 inhibitors, dna methyltransferase inhibitors) also induce p21, and have been shown to have activity in patients with cancer. Thus, p21 induction is a plausable marker of anticancer potential when a target is appropriately modulated.

In preferred embodiments, HGPRBMY8 polynucleotides and polypeptides, including fragments thereof, are useful for treating, diagnosing, and/or ameliorating cell cycle defects, disorders related to aberrant phosphorylation, disorders related to aberrant signal transduction, proliferating disorders, and/or cancers.

Moreover, HGPRBMY8 polynucleotides and polypeptides, including fragments thereof, are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

In preferred embodiments, agonists directed to HGPRBMY8 are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

HGPRBMY8 polynucleotides and polypeptides, including fragments and agonists thereof, are useful for treating, preventing, or ameliorating proliferative disorders in a patient in need of treatment, such as cancer patients, particularly patients that have proliferative immune disorders such as leukemia, lymphomas, multiple myeloma, etc.

Moreover, antagonists directed against HGPRBMY8 are useful for increasing cellular proliferation, increasing cellular proliferation in rapidly proliferating cells, decreasing the number of cells in the G1 phase of the cell cycle, decreasing the number of cells in the G2 phase of the cell cycle, increasing the number of cells that progress to the S phase of the cell cycle, increasing the number of cells that progress to the M phase of the cell cycle, and releasing cells from G1 and/or G2 phase arrest. Such antagonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc. In addition, such antagonists of HGPRBMY8 would also be useful for regenerating neural tissues (e.g., treatment of Parkinson's or Alzheimers patients with neural stem cells, or neural cells which have been activated by an HGPRBMY8 antagonist).

Characterization of the HGPRBMY8 polypeptide of the present invention using antisense oligonucleotides led to the determination that HGPRBMY8 is involved in the positive modulation of the p27 G1/G2 cell cycle check point modulatory protein as described in Example 17 herein.

Antisense oligonucleotides directed against the HGPRBMY8 mRNA resulted in a marked decrease in p27 expression and/or activity (see Example 17). The level of expression of p27 in response to treatment with antisense specific to HGPRBMY8 was decreased by about 43%. The results were replicated in three independent experiments and determined to be statistically significant.

In preferred embodiments, HGPRBMY8 polynucleotides and polypeptides, including modulators and fragments thereof, are useful for treating, diagnosing, and/or ameliorating cell cycle defects, disorders related to aberrant phosphorylation, disorders related to aberrant signal transduction, proliferating disorders, and/or cancers.

Moreover, HGPRBMY8 polynucleotides and polypeptides, including modulators and fragments thereof, are useful for decreasing cellular proliferation, decreasing cellular proliferation in rapidly proliferating cells, increasing the number of cells in the G1 phase of the cell cycle, and decreasing the number of cells that progress to the S phase of the cell cycle.

In preferred embodiments, agonists directed to HGPRBMY8 are useful for decreasing cellular proliferation, decreasing cellular proliferation in rapidly proliferating cells, increasing the number of cells in the G1 phase of the cell cycle, and decreasing the number of cells that progress to the S phase of the cell cycle.

Moreover, antagonists directed against HGPRBMY8 are useful for increasing cellular proliferation, increasing cellular proliferation in rapidly proliferating cells, decreasing the number of cells in the G1 phase of the cell cycle, and increasing the number of cells that progress to the S phase of the cell cycle. Such agonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc. In addition, such agonists of HGPRBMY8 would also be useful for increasing male fertility, correcting or ameliorating endocrine deficiencies or growth disorders, and in regenerating neural tissues (e.g., treatment of Parkinson's or Alzheimers patients with neural stem cells, or neural cells which have been activated by an HGPRBMY8 agonist).

Characterization of the HGPRBMY8 polypeptide of the present invention using antisense oligonucleotides led to the determination that HGPRBMY8 is involved in modulation of the NFKB pathway through the positive modulation of the IkB modulatory protein as described in Example 17 herein.

Antisense oligonucleotides directed against the HGPRBMY8 mRNA resulted in a marked decrease in IkB expression and/or activity (see Example 17). The level of expression of IkB in response to treatment with antisense specific to HGPRBMY8 was decreased by about 60%. The results were replicated in three independent experiments and determined to be statistically significant.

In preferred embodiments, HGPRBMY8 polynucleotides and polypeptides, including modulators and fragments thereof, are useful for treating, diagnosing, and/or ameliorating proliferative disorders, cancers, ischemia-reperfusion injury, heart failure, immuno compromised conditions, HIV infection, and renal diseases.

Moreover, HGPRBMY8 polynucleotides and polypeptides, including modulators and fragments thereof, are useful for increasing NF-kB activity, decreasing apoptotic events, and/or decreasing IκBα expression or activity levels.

In preferred embodiments, agonists directed against HGPRBMY8 are useful for treating, diagnosing, and/or ameliorating autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, and HIV propagation in cells infected with other viruses.

Moreover, agonists directed against HGPRBMY8 are useful for decreasing NF-kB activity, increasing apoptotic events, and/or increasing IκBα expression or activity levels.

In preferred embodiments, antagonists directed against HGPRBMY8 are useful for treating, diagnosing, and/or ameliorating autoimmune diorders, disorders related to hyper immune activity, hypercongenital conditions, birth defects, necrotic lesions, wounds, disorders related to aberrant signal transduction, immuno compromised conditions, HIV infection, proliferating disorders, and/or cancers.

Moreover, antagonists directed against HGPRBMY8 are useful for increasing NF-kB activity, decreasing apoptotic events, and/or decreasing IκBα expression or activity levels.

Collectively, the antisense results for this target appears to provide mixed results on first impression. However, signficant downregulation of P27 and upregulation of P21 suggests that inhibition of HGPRBMY8 would induce proliferation, in addition to inducing differentiation and cell cycle inhibition. Suppression of IkB, as a marker for suppression of apoptosis in consistent with induction of proliferation and differentiation as well as cell cycle inhibition. Based upon the results, it is possible that increases in P21 are compensatory for inhibition of the target, and that agonists of this target would be most preferred in order to induce P27 and IkB expression. Likewise, the present invention is preferably directed to an agonist of HGPRBMY8 for the treatment of cancers and/or proliferative conditions.

The strong homology to human G-protein coupled receptors, combined with the localized expression in nucleus accumbens and caudate nucleus, suggests the HGPRBMY8 polynucleotides and polypeptides, including fragments, variants, and modulators thereof, may be useful in treating, diagnosing, prognosing, and/or preventing neurodegenerative disease states, and behavioral disorders. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

Also encompassed by the present invention are polynucleotide sequences that are capable of hybridizing to the claimed nucleotide sequence of HGPRBMY8, such as that shown in SEQ ID NO:1, under various conditions of stringency. Hybridization conditions are typically based on the melting temperature (Tₘ) of the nucleic acid binding complex or probe (see, G. M. Wahl and S. L. Berger, 1987; Methods Enzmmol., 152:399-407 and A. R. Kimmel, 1987; Methods of Enzmmol., 152:507-511), and may be used at a defined stringency. For example, included in the present invention are sequences capable of hybridizing under moderately stringent conditions to the HGPRBMY8 sequence of SEQ ID NO:1 and other sequences which are degenerate to those which encode HGPRBMY8 polypeptide (e.g., as a non-limiting example: prewashing solution of 2×SSC, 0.5% SDS, 1.0 mM EDTA, pH 8.0, and hybridization conditions of 50° C., 5×SSC, overnight.

The nucleic acid sequence encoding the HGPRBMY8 protein may be extended utilizing a partial nucleotide sequence and employing various methods known in the art to detect upstream sequences such as promoters and regulatory elements. For example, one method, which may be employed, is restriction-site PCR, which utilizes universal primers to retrieve unknown sequence adjacent to a known locus (G. Sarkar, 1993, PCR Methods Applic., 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences Inc.; Plymouth, Minn.), or another appropriate program, to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. In addition, PCR, nested primers, and PROMOTERFINDER libraries can be used to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, since they will contain more sequences, which contain the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp.; Cleveland, Ohio), Taq polymerase (PE Biosystems; Gaithersburg, Md.), thermostable T7 polymerase (Amersham Pharmacia Biotechnology; Piscataway, N.J.), or combinations of recombinant polymerases and proofreading exonucleases such as the ELONGASE Amplification System marketed by Life Technologies (Rockville, Md.). Preferably, the process is automated with machines such as the Hamilton Micro Lab 2200 (Hamilton; Reno, Nev.), Peltier Thermal Cycler (PTC200; M J Research; Watertown, Mass.) and the ABI Catalyst and 373 and 377 DNA sequencers (PE Biosystems; Gaithersburg, Md.).

Commercially available capillary electrophoresis systems may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGATOR, PE Biosystems; Gaithersburg, Md.) and the entire process—from loading of samples to computer analysis and electronic data display—may be computer controlled. Capillary electrophoresis is especially preferable for the sequencing of small pieces of DNA, which might be present in limited amounts in a particular sample.

In another embodiment of the present invention, polynucleotide sequences or fragments thereof which encode HGPRBMY8 polypeptide, or peptides thereof, may be used in recombinant DNA molecules to direct the expression of HGPRBMY8 polypeptide product, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express HGPRBMY8 protein.

As will be appreciated by those having skill in the art, it may be advantageous to produce HGPRBMY8 polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequence of the present invention can be engineered using methods generally known in the art in order to alter HGPRBMY8 polypeptide-encoding sequences for a variety of reasons, including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In another embodiment of the present invention, natural, modified, or recombinant nucleic acid sequences encoding HGPRBMY8 polypeptide may be ligated to a heterologous sequence to encode a fusion protein. For example, for screening peptide libraries for inhibitors of HGPRBMY8 activity, it may be useful to encode a chimeric HGPRBMY8 protein that can be recognized by a commercially available antibody. A fusion protein may also be engineered to contain a cleavage site located between the HGPRBMY8 protein-encoding sequence and the heterologous protein sequence, so that HGPRBMY8 protein may be cleaved and purified away from the heterologous moiety.

In another embodiment, sequences encoding HGPRBMY8 polypeptide may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the protein itself may be produced using chemical methods to synthesize the amino acid sequence of HGPRBMY8 polypeptide, or a fragment or portion thereof. For example, peptide synthesis can be performed using various solid-phase techniques (J. Y. Roberge et al., 1995, Science, 269:202-204) and automated synthesis may be achieved, for example, using the ABI 431A Peptide Synthesizer (PE Biosystems; Gaithersburg, Md.).

The newly synthesized peptide can be substantially purified by preparative high performance liquid chromatography (e.g., T. Creighton, 1983, Proteins, Structures and Molecular Principles, W. H. Freeman and Co., New York, N.Y.), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of HGPRBMY8 polypeptide or any portion thereof, may be altered during direct synthesis and/or combined using chemical methods with sequences from other proteins, or any part thereof, to produce a variant polypeptide.

To express a biologically active HGPRBMY8 polypeptide or peptide, the nucleotide sequences encoding HGPRBMY8 polypeptide, or functional equivalents, may be inserted into an appropriate expression vector, i.e., a vector, which contains the necessary elements for the transcription and translation of the inserted coding sequence.

Methods, which are well known to those skilled in the art, may be used to construct expression vectors containing sequences encoding HGPRBMY8 polypeptide and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/ host systems may be utilized to contain and express sequences encoding HGPRBMY8 polypeptide. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems. The host cell employed is not limiting to the present invention.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene; La Jolla, Calif.) or PSPORT1 plasmid (Life Technologies; Rockville, Md.), and the like, may be used. The baculovirus polyhedrin promoter may be used in insect cells. Promoters or enhancers derived from the genomes of plant cells (e.g., heat shock, RUBISCO; and storage protein genes), or from plant viruses (e.g., viral promoters or leader sequences), may be cloned into the vector. In mammalian cell systems, promoters from mammalian genes or from mammalian viruses are preferred. If it is necessary to generate a cell line that contains multiple copies of the sequence encoding HGPRBMY8, vectors based on SV40 or EBV may be used with an appropriate selectable marker.

In bacterial systems, a number of expression vectors may be selected, depending upon the use intended for the expressed HGPRBMY8 product. For example, when large quantities of expressed protein are needed for the induction of antibodies, vectors, which direct high level expression of fusion proteins that are readily purified, may be used. Such vectors include, but are not limited to, the multifunctional E. coli cloning and expression vectors such as BLUESCRIPT (Stratagene; La Jolla, Calif.), in which the sequence encoding HGPRBMY8 polypeptide may be ligated into the vector in-frame with sequences for the amino-terminal Met and the subsequent 7 residues of 13-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) may also be used to express foreign polypeptides, as fusion proteins with glutathione S-transferase (GST). In general, such fusion proteins are soluble and can be easily purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (For reviews, see F. M. Ausubel et al., supra, and Grant et al., 1987, Methods Enzymol., 153:516-544).

Should plant expression vectors be desired and used, the expression of sequences encoding HGPRBMY8 polypeptide may be driven by any of a number of promoters. For example, viral promoters such as the 35S and 19S promoters of CaMV may be used alone or in combination with the omega leader sequence from TMV (N. Takamatsu, 1987, EMBO J., 6:307-311). Alternatively, plant promoters such as the small subunit of RUBISCO, or heat shock promoters, may be used (G. Coruzzi et al., 1984, EMBO J., 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (see, for example, S. Hobbs or L. E. Murry, In: McGraw Hill Yearbook of Science and Technology (1992) McGraw Hill, New York, N.Y.; pp. 191-196).

An insect system may also be used to express HGPRBMY8 polypeptide. For example, in one such system, Autographa californica nuclear polyhedrosis virus (ACNPV) is used as a vector to express foreign genes in Spodoptera frugiperda cells or in Trichoplusia larvae. The sequences encoding HGPRBMY8 polypeptide may be cloned into a non-essential region of the virus such as the polyhedrin gene and placed under control of the polyhedrin promoter. Successful insertion of HGPRBMY8 polypeptide will render the polyhedrin gene inactive and produce recombinant virus lacking coat protein. The recombinant viruses may then be used to infect, for example, S. frugiperda cells or Trichoplusia larvae in which the HGPRBMY8 polypeptide product may be expressed (E. K. Engelhard et al., 1994, Proc. Nat. Acad. Sci., 91:3224-3227).

In mammalian host cells, a number of viral-based expression systems may be utilized. In cases where an adenovirus is used as an expression vector, sequences encoding HGPRBMY8 polypeptide may be ligated into an adenovirus transcription/ translation complex containing the late promoter and tripartite leader sequence. Insertion in a non-essential E1 or E3 region of the viral genome may be used to obtain a viable virus which is capable of expressing HGPRBMY8 polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding HGPRBMY8 polypeptide. Such signals include the ATG initiation codon and adjacent sequences. In cases where sequences encoding HGPRBMY8 polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals, including the ATG initiation codon, should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system that is used, such as those described in the literature (D. Scharf et al., 1994, Results Probl. Cell Differ., 20:125-162).

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post- translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and may be chosen to ensure the correct modification and processing of the foreign protein.

For long-term, high-yield production of recombinant proteins, stable expression is preferred. For example, cell lines which stably express HGPRBMY8 protein may be transformed using expression vectors which may contain viral origins of replication and/or endogenous expression elements and a selectable marker gene on the same, or on a separate, vector. Following the introduction of the vector, cells may be allowed to grow for 1-2 days in an enriched cell culture medium before they are switched to selective medium. The purpose of the selectable marker is to confer resistance to selection, and its presence allows the growth and recovery of cells, which successfully express the introduced sequences. Resistant clones of stably transformed cells may be proliferated using tissue culture techniques appropriate to the cell type.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding HGPRBMY8 polypeptide is inserted within a marker gene sequence, recombinant cells containing sequences encoding HGPRBMY8 polypeptide can be identified by the absence of marker gene function. Alternatively, a marker gene can be placed in tandem with a sequence encoding HGPRBMY8 polypeptide under the control of a single promoter. Expression of the marker gene in response to induction or selection usually indicates co-expression of the tandem gene.

Alternatively, host cells, which contain the nucleic acid, sequence encoding HGPRBMY8 polypeptide and which express HGPRBMY8 polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding HGPRBMY8 polypeptide can be detected by DNA-DNA or DNA-RNA hybridization, or by amplification using probes or portions or fragments of polynucleotides encoding HGPRBMY8 polypeptide. Nucleic acid amplification based assays involve the use of oligonucleotides or oligomers, based on the sequences encoding HGPRBMY8 polypeptide, to detect transformants containing DNA or RNA encoding HGPRBMY8 polypeptide.

A wide variety of labels and conjugation techniques are known and employed by those skilled in the art and may be used in various nucleic acid and amino acid assays. Means for producing labeled hybridization or PCR probes for detecting sequences related to polynucleotides encoding HGPRBMY8 polypeptide include oligo-labeling, nick translation, end-labeling, or PCR amplification using a labeled nucleotide. Alternatively, the sequences encoding HGPRBMY8 polypeptide, or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which may be used include radionucleotides, enzymes, fluorescent, chemiluminescent, or chromogenic agents, as well as substrates, cofactors, inhibitors, magnetic particles, and the like.

Furthermore, in yet another embodiment, G-protein coupled receptor-encoding polynucleotide sequences can be used to purify a molecule or compound in a sample, wherein the molecule or compound specifically binds to the polynucleotide, comprising: a) combining the G-protein coupled receptor-encoding polynucleotide, or fragment thereof, under conditions to allow specific binding; b) detecting specific binding between the G-protein coupled receptor-encoding polynucleotide and the molecule or compound; c) recovering the bound polynucleotide; and d) separating the polynucleotide from the molecule or compound, thereby obtaining a purified molecule or compound.

Host cells transformed with nucleotide sequences encoding HGPRBMY8 protein, or fragments thereof, may be cultured under conditions suitable for the expression and recovery of the protein from cell culture. The protein produced by a recombinant cell may be secreted or contained intracellularly depending on the sequence and/ or the vector used. As will be understood by those having skill in the art, expression vectors containing polynucleotides which encode HGPRBMY8 protein may be designed to contain signal sequences which direct secretion of the HGPRBMY8 protein through a prokaryotic or eukaryotic cell membrane. Other constructions may be used to join nucleic acid sequences encoding HGPRBMY8 protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/ affinity purification system (Immunex Corp., Seattle, Wash.). The inclusion of cleavable linker sequences such as those specific for Factor XA or enterokinase (Invitrogen, San Diego, Calif.) between the purification domain and HGPRBMY8 protein may be used to facilitate purification. One such expression vector provides for expression of a fusion protein containing HGPRBMY8 and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying from the fusion protein. For a discussion of suitable vectors for fusion protein production, see D.J. Kroll et al., 1993; DNA Cell Biol., 12:441-453.

In addition to recombinant production, fragments of HGPRBMY8 polypeptide may be produced by direct peptide synthesis using solid-phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems; Gaithersburg, Md.). Various fragments of HGPRBMY8 polypeptide can be chemically synthesized separately and then combined using chemical methods to produce the full-length molecule.

Human artificial chromosomes (HACs) may be used to deliver larger fragments of DNA than can be contained and expressed in a plasmid vector. HACs are linear microchromosomes which may contain DNA sequences of 10K to 10M in size, and contain all of the elements that are required for stable mitotic chromosome segregation and maintenance (see, J. J. Harrington et al., 1997, Nature Genet., 15:345). HACs of 6 to 10M are constructed and delivered via conventional delivery methods (e.g., liposomes, polycationic amino polymers, or vesicles) for therapeutic purposes.

### Diagnostic Assays

A variety of protocols for detecting and measuring the expression of HGPRBMY8 polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the HGPRBMY8 polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med. 158:1211-1216).

This invention also relates to the use of HGPRBMY8 polynucleotides as diagnostic reagents. Detection of a mutated form of the HGPRBMY8 gene associated with a dysfunction provides a diagnostic tool that can add to or define a diagnosis of a disease or susceptibility to a disease which results from under-expression, over- expression, or altered expression of HGPRBMY8. Individuals carrying mutations in the HGPRBMY8 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Hybridizing amplified DNA to labeled HGPRBMY8 polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and S1 protection or the chemical cleavage method. See Cotton et al., Proc. Natl. Acad. Sci., USA (1985) 85:43297-4401. In another embodiment, an array of oligonucleotides probes comprising HGPRBMY8 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, 274:610-613, 1996).

The diagnostic assays offer a process for diagnosing or determining, for example, a susceptibility to infections such as bacterial, fungal, protozoan and viral infections, particularly infections caused by HIV-1 or HIV-2 through detection of a mutation in the HGPRBMY8 gene by the methods described. The invention also provides diagnostic assays for determining or monitoring susceptibility to the following conditions, diseases, or disorders: HIV infections; asthma; allergies; obesity; anorexia; bulimia; ulcers; acute heart failure; hypotension; hypertension; angina pectoris; myocardial infarction; urinary retention; osteoporosis; benign prostatic hypertrophy; cancers; brain-related disorders; Parkinson's disease; neuropathic pain; immune; metabolic; cardiovascular; and psychotic and neurological disorders, including anxiety, schizophrenia, manic depression, delirium, dementia, severe mental retardation and dyskinesias, such as Huntington's disease or Gilles dela Tourett's syndrome; Sydenham chorea; major depressive disorder; and obsessive- compulsive disorder (OCD). Movement type diseases, disorders, or conditions may be targeted in particular since HGPRBMY8 is expressed in the caudate nucleus of the brain.

In addition, infections such as bacterial, fungal, protozoan and viral infections, particularly infections caused by HIV-1 or HIV-2, as well as, conditions, diseases, or disorders such as, HIV infections; asthma; allergies; obesity; anorexia; bulimia; ulcers; acute heart failure; hypotension; hypertension; angina pectoris; myocardial infarction; urinary retention; osteoporosis; benign prostatic hypertrophy; cancers; brain-related disorders; Parkinson's disease; neuropathic pain; immune; metabolic; cardiovascular; and psychotic and neurological disorders, including anxiety, schizophrenia, manic depression, delirium, dementia, severe mental retardation and dyskinesias, such as Huntington's disease or Gilles dela Tourett's syndrome, can be diagnosed by methods comprising determining from a sample derived from a subject having an abnormally decreased or increased level of HGPRBMY8 polypeptide or HGPRBMY8 mRNA. Decreased or increased expression can be measured at the RNA level using any of the methods well known in the art for the quantification of polynucleotides, such as, for example, PCR, RT-PCR, RNAse protection, Northern blotting and other hybridization methods. Assay techniques that can be used to determine levels of a protein, such as an HGPRBMY8, in a sample derived from a host are well known to those of skill in the art. Such assay methods include radioimmunoassays, competitive-binding assays, Western Blot analysis and ELISA assays.

In another of its aspects, the present invention relates to a diagnostic kit for a disease or susceptibility to a disease, particularly infections such as bacterial, fungal, protozoan and viral infections, particularly infections caused by HIV-1 or HIV-2, as well as, conditions, diseases, or disorders such as, HIV infections; asthma; allergies; obesity; anorexia; bulimia; ulcers; acute heart failure; hypotension; hypertension; angina pectoris; myocardial infarction; urinary retention; osteoporosis; benign prostatic hypertrophy; cancers; brain-related disorders; Parkinson's disease; neuropathic pain; immune; metabolic; cardiovascular; and psychotic and neurological disorders, including anxiety, schizophrenia, manic depression, delirium, dementia, severe mental retardation and dyskinesias, such as Huntington's disease or Gilles dela Tourett's syndrome, which comprises:

(a) an HGPRBMY8 polynucleotide, preferably the nucleotide sequence of SEQ ID NO:1, or a fragment thereof; or

(b) a nucleotide sequence complementary to that of (a); or

(c) an HGPRBMY8 polypeptide, preferably the polypeptide of SEQ ID NO: 2, or a fragment thereof; or

(d) an antibody to an HGPRBMY8 polypeptide, preferably to the polypeptide of SEQ ID) NO: 2, or combinations thereof.

It will be appreciated that in any such kit, (a), (b), (c) or (d) may comprise a substantial component and instructions are frequently included.

The GPCR polynucleotides which may be used in the diagnostic assays according to the present invention include oligonucleotide sequences, complementary RNA and DNA molecules, and PNAs. The polynucleotides may be used to detect and quantify HGPRBMY8-encoding nucleic acid expression in biopsied tissues in which expression (or under- or overexpression) of the HGPRBMY8 polynucleotide may be correlated with disease. The diagnostic assays may be used to distinguish between the absence, presence, and excess expression of HGPRBMY8, and to monitor regulation of HGPRBMY8 polynucleotide levels during therapeutic treatment or intervention.

In a related aspect, hybridization with PCR probes which are capable of detecting polynucleotide sequences, including genomic sequences, encoding HGPRBMY8 polypeptide, or closely related molecules, may be used to identify nucleic acid sequences which encode HGPRBMY8 polypeptide. The specificity of the probe, whether it is made from a highly specific region, e.g., about 8 to 10 contiguous nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding HGPRBMY8 polypeptide, alleles thereof, or related sequences.

Probes may also be used for the detection of related sequences, and should preferably contain at least 50% of the nucleotides, most optimally 15-35 nucleotides, encoding the HGPRBMY8 polypeptide. The hybridization probes of this invention may be DNA or RNA and may be derived from the nucleotide sequence of SEQ ID NO:1, or from genomic sequence including promoter, enhancer elements, and introns of the naturally occurring HGPRBMY8 protein.

Methods for producing specific hybridization probes for DNA encoding the HGPRBMY8 polypeptide include the cloning of a nucleic acid sequence that encodes the HGPRBMY8 polypeptide, or HGPRBMY8 derivatives, into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of detector/ reporter groups, e.g., radionuclides such as ³²P or ³⁵S, or enzymatic labels, such as alkaline phosphatase coupled to the probe via avidin/ biotin coupling systems, and the like.

The polynucleotide sequence encoding the HGPRBMY8 polypeptide, or fragments thereof, may be used for the diagnosis of disorders associated with expression of HGPRBMY8. Examples of such disorders or conditions are described for “Therapeutics”. The polynucleotide sequence encoding the HGPRBMY8 polypeptide may be used in Southern or Northern analysis, dot blot, or other membrane-based technologies; in PCR technologies; or in dip stick, pin, ELISA or chip assays utilizing fluids or tissues from patient biopsies to detect the status of, e.g., levels or overexpression of HGPRBMY8, or to detect altered HGPRBMY8 expression. Such qualitative or quantitative methods are well known in the art.

In a particular aspect, the nucleotide sequence encoding the HGPRBMY8 polypeptide may be useful in assays that detect activation or induction of various neoplasms or cancers, particularly those mentioned supra. The nucleotide sequence encoding the HGPRBMY8 polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the HGPRBMY8 polypeptide in the sample indicates the presence of the associated disease. Such assays may also be used to evaluate the efficacy of a particular therapeutic treatment regimen in animal studies, in clinical trials, or in monitoring the treatment of an individual patient.

To provide a basis for the diagnosis of disease associated with expression of HGPRBMY8, a normal or standard profile for expression is established. This may be accomplished by combining body fluids or cell extracts taken from normal subjects, either animal or human, with a sequence, or a fragment thereof, which encodes the HGPRBMY8 polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject (patient) values is used to establish the presence of disease.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to cancer, the presence of an abnormal amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the nucleic acid sequence encoding the HGPRBMY8 polypeptide may involve the use of PCR. Such oligomers may be chemically synthesized, generated enzymatically, or produced from a recombinant source. Oligomers will preferably comprise two nucleotide sequences, one with sense orientation (5′→3′) and another with antisense (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of HGPRBMY8 include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or calorimetric response gives rapid quantification.

### Therapeutic Assays

The HGPRBMY8 polypeptide (SEQ ID NO:2) shares homology with somatostatin-type receptors. The HGPRBMY8 protein may play a role in neurological disorders, and/or in cell cycle regulation, and/or in cell signaling. The HGPRBMY8 protein may further be involved in neoplastic, cardiovascular, and immunological disorders.

In one embodiment of the present invention, the HGPRBMY8 protein may play a role in neoplastic disorders. An antagonist or inhibitor of the HGPRBMY8 polypeptide may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to HGPRBMY8 may be used directly as an antagonist or indirectly as a targeting or delivery mechanism for bringing a pharmaceutical agent to cells or tissue which express the HGPRBMY8 polypeptide.

In another embodiment of the present invention, an antagonist or inhibitory agent of the HGPRBMY8 polypeptide may be administered to an individual to prevent or treat an immunological disorder. Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections and trauma.

In a preferred embodiment of the present invention, an antagonist or inhibitory agent of the HGPRBMY8 polypeptide may be administered to an individual to prevent or treat a neurological disorder, particularly since HGPRBMY8 is highly expressed in the brain. Such disorders may include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In preferred embodiments, the HGPRBMY8 polynucleotides and polypeptides, including agonists, antagonists, and fragments thereof, are useful for modulating intracellular cAMP associated signaling pathways.

In another embodiment of the present invention, an expression vector containing the complement of the polynucleotide encoding HGPRBMY8 polypeptide may be administered to an individual to treat or prevent a neoplastic disorder, including, but not limited to, the types of cancers and tumors described above.

In yet another embodiment of the present invention, an expression vector containing the complement of the polynucleotide encoding HGPRBMY8 polypeptide may be administered to an individual to treat or prevent an immune disorder, including, but not limited to, the types of immune disorders described above.

In a preferred embodiment of the present invention, an expression vector containing the complement of the polynucleotide encoding HGPRBMY8 polypeptide may be administered to an individual to treat or prevent a neurological disorder, including, but not limited to, the types of disorders described above.

In another embodiment, the proteins, antagonists, antibodies, agonists, complementary sequences, or vectors of the present invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of the HGPRBMY8 polypeptide of the present invention may be produced using methods which are generally known in the art. For example, the HGPRBMY8 transfected CHO-NFAT/CRE cell lines of the present invention are useful for the identification of agonists and antagonists of the HGPRBMY8 polypeptide. Representative uses of these cell lines would be their inclusion in a method of identifying HGPRBMY8 agonists and antagonists. Preferably, the cell lines are useful in a method for identifying a compound that modulates the biological activity of the HGPRBMY8 polypeptide, comprising the steps of (a) combining a candidate modulator compound with a host cell expressing the HGPRBMY8 polypeptide having the sequence as set forth in SEQ ID NO:2; and (b) measuring an effect of the candidate modulator compound on the activity of the expressed HGPRBMY8 polypeptide. Representative vectors expressing the HGPRBMY8 polypeptide are referenced herein (e.g., pcDNA3.1 hygro™) or otherwise known in the art.

The cell lines are also useful in a method of screening for a compounds that is capable of modulating the biological activity of HGPRBMY8 polypeptide, comprising the steps of: (a) determining the biological activity of the HGPRBMY8 polypeptide in the absence of a modulator compound; (b) contacting a host cell expression the HGPRBMY8 polypeptide with the modulator compound; and (c) determining the biological activity of the HGPRBMY8 polypeptide in the presence of the modulator compound; wherein a difference between the activity of the HGPRBMY8 polypeptide in the presence of the modulator compound and in the absence of the modulator compound indicates a modulating effect of the compound. Additional uses for these cell lines are described herein or otherwise known in the art. In particular, purified HGPRBMY8 protein, or fragments thereof, can be used to produce antibodies, or to screen libraries of pharmaceutical agents, to identify those which specifically bind HGPRBMY8.

Antibodies specific for HGPRBMY8 polypeptide, or immunogenic peptide fragments thereof, can be generated using methods that have long been known and conventionally practiced in the art. Such antibodies may include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use.

The present invention also encompasses the polypeptide sequences that intervene between each of the predicted HGPRBMY8 transmembrane domains. Since these regions are solvent accessible either extracellularly or intracellularly, they are particularly useful for designing antibodies specific to each region. Such antibodies may be useful as antagonists or agonists of the HGPRBMY8 full-length polypeptide and may modulate its activity.

The following serve as non-limiting examples of peptides or fragments that may be used to generate antibodies:

| | |
|---|---|
| MTSTCTNSTRESNSSHTCMPLSKMPISLAHGIIRST | (SEQ ID NO:26) |
| QRKPQLLQVTNRF | (SEQ ID NO:27) |
| WPLNS | (SEQ ID NO:28) |
| DRYLSIIHPLSYPSKMTQRR | (SEQ ID NO:29) |
| GQAAFDERNALCSMIWGASPSYT | (SEQ ID NO:30) |
| CAARRQHALLYNVKRHSLEVRVKDCVENEDEEGAEK | (SEQ ID NO:31) |
| KEEFQDESEFRRQHEGEVKAKEGRMEAKDGSLKAKE | |
| GSTGTSESSVEAGSEEVRESSTVASDGSMEGKEGST | |
| KVEENSMKADKGRTEVNQCSIDLGEDDMEFGEDDIN | |
| FSEDDVEAVNIPESLPPSRRNSNSNPPLPRCYQCKA | |
| AK | |
| AVLAVWVDVETQVPQ | (SEQ ID NO:32) |
| YGYMHKTIKKEIQDMLKKFFCKEKPPKEDSHPDLPG | (SEQ ID NO:33) |
| TEGGTEGKIVPSYDSATFP | |

The present invention also encompasses the polypeptide sequences that intervene between each of the predicted HGPRBMY8 transmembrane domains. Since these regions are solvent accessible either extracellularly or intracellularly, they are particularly useful for designing antibodies specific to each region. Such antibodies may be useful as antagonists or agonists of the HGPRBMY8 full-length polypeptide and may modulate its activity.

In preferred embodiments, the following N-terminal HGPRBMY8 TM1-2 intertransmembrane domain deletion polypeptides are encompassed by the present invention: Q1-F13, R2-F13, K3-F13, P4-F13, Q5-F13, L6-F13, and/or L7-F13 of SEQ ID NO:27. Polynucleotide sequences encoding these polypeptides are also provided. The present invention also encompasses the use of these N-terminal HGPRBMY8 TM1-2 intertransmembrane domain deletion polypeptides as immunogenic and/or antigenic epitopes as described elsewhere herein.

In preferred embodiments, the following C-terminal HGPRBMY8 TM1-2 intertransmembrane domain deletion polypeptides are encompassed by the present invention: Q1-F13, Q1-R12, Q1-NI1, Q1-T10, Q1-V9, Q1-Q8, and/or Q1-L7 of SEQ ID NO:27. Polynucleotide sequences encoding these polypeptides are also provided. The present invention also encompasses the use of these C-terminal HGPRBMY8 TM1-2 intertransmembrane domain deletion polypeptides as immunogenic and/or antigenic epitopes as described elsewhere herein.

In preferred embodiments, the following N-terminal HGPRBMY8 TM3-4 intertransmembrane domain deletion polypeptides are encompassed by the present invention: D1-R20, R2-R20, Y3-R20, L4-R20, S5-R20, 16-R20, 17-R20, H8-R20, P9-R20, L10-R20, S11-R20, Y12-R20, P13-R20, and/or S14-R20 of SEQ ID NO:29. Polynucleotide sequences encoding these polypeptides are also provided. The present invention also encompasses the use of these N-terminal HGPRBMY8 TM3-4 intertransmembrane domain deletion polypeptides as immunogenic and/or antigenic epitopes as described elsewhere herein.

In preferred embodiments, the following C-terminal HGPRBMY8 TM3-4 intertransmembrane domain deletion polypeptides are encompassed by the present invention: D1-R20, D1-R19, D1-Q18, D1-T17, D1-M16, D1-KI5, D1-S14, D1-P13, D1-Y12, D1-S1, D1-L10, D1-P9, D1-H8, and/or D1-17 of SEQ ID NO:29. Polynucleotide sequences encoding these polypeptides are also provided. The present invention also encompasses the use of these C-terminal HGPRBMY8 TM3-4 intertransmembrane domain deletion polypeptides as immunogenic and/or antigenic epitopes as described elsewhere herein.

In preferred embodiments, the following N-terminal HGPRBMY8 TM4-5 intertransmembrane domain deletion polypeptides are encompassed by the present invention: G1-T23, Q2-T23, A3-T23, A4-T23, F5-T23, D6-T23, E7-T23, R8-T23, N9-T23, A10-T23, L11-T23, C12-T23, S13-T23, M14-T23, 115-T23, W16-T23, and/or G17-T23 of SEQ ID NO:30. Polynucleotide sequences encoding these polypeptides are also provided. The present invention also encompasses the use of these N-terminal HGPRBMY8 TM4-5 intertransmembrane domain deletion polypeptides as immunogenic and/or antigenic epitopes as described elsewhere herein.

In preferred embodiments, the following C-terminal HGPRBMY8 TM4-5 intertransmembrane domain deletion polypeptides are encompassed by the present invention: G1-T23, G1-Y22, G1-S21, G1-P20, G1-S19, G1-A18, G1-G17, G1-W16, G1-115, G1-M14, G1-S13, G1-C12, G1-L11, G1-A10, G1-N9, G1-R8, and/or G1-E7 of SEQ ID NO:30. Polynucleotide sequences encoding these polypeptides are also provided. The present invention also encompasses the use of these C-terminal HGPRBMY8 TM4-5 intertransmembrane domain deletion polypeptides as immunogenic and/or antigenic epitopes as described elsewhere herein.

In preferred embodiments, the following N-terminal HGPRBMY8 TM5-6 intertransmembrane domain deletion polypeptides are encompassed by the present invention: C1-K182, A2-K182, A3-K182, R4-K182, R5-K182, Q6-K182, H7-K182, A8-K182, L9-K182, L10-K182, Y11-K182, N12-K182, V13-K182, K14-K182, R15-K182, H16-K182, S17-K182, L18-K182, E19-K182, V20-K182, R21-K182, V22-K182, K23-K182, D24-K182, C25-K182, V26-K182, E27-K182, N28-K182, E29-K182, D30-K182, E31-K182, E32-K182, G33-K182, A34-K182, E35-K182, K36-K182, K37-K182, E38-K182, E39-K182, F40-K182, Q41-K182, D42-K182, E43-K182, S44-K182, E45-K182, F46-K182, R47-K182, R48-K182, Q49-K182, H50-K182, E51-K182, G52-K182, E53-K182, V54-K182, K55-K182, A56-K182, K57-K182, E58-K182, G59-K182, R60-K182, M61-K182, E62-K182, A63-K182, K64-K182, D65-K182, G66-K182, S67-K182, L68-K182, K69-K182, A70-K182, K71-K182, E72-K182, G73-K182, S74-K182, T75-K182, G76-K182, T77-K182, S78-K182, E79-K182, S80-K182, S81-K182, V82-K182, E83-K182, A84-K182, G85-K182, S86-K182, E87-K182, E88-K182, V89-K182, R90-K182, E91-K182, S92-K182, S93-K182, T94-K182, V95-K182, A96-K182, S97-K182, D98-K182, G99-K182, S100-K182, M101-K182, E102-K182, G103-K182, K104-K182, E105-K182, G106-K182, S107-K182, T108-K182, K109-K182, V110-K182, E111-K182, E112-K182, N113-K182, S114-K182, M115-K182, K116-K182, A117-K182, D118-K182, K119-K182, G120-K182, R121-K182, T122-K182, E123-K182, V124-K182, N125-K182, Q126-K182, C127-K182, S128-K182, 1129-K182, D130-K182, L131-K182, G132-K182, E133-K182, D134-K182, D135-K182, M136-K182, E137-K182, F138-K182, G139-K182, E140-K182, D141-K182, D142-K182, 1143-K182, N144-K182, F145-K182, S146-K182, E147-K182, D148-K182, D149-K182, V150-K182, E151-K182, A152-K182, V153-K182, N154-K182, 1155-K182, P156-K182, E157-K182, S158-K182, L159-K182, P160-K182, P161-K182, S162-K182, R163-K182, R164-K182, N165-K182, S166-K182, N167-K182, S168-K182, N169-K182, P170-K182, P171-K182, L172-K182, P173-K182, R174-K182, C175-K182, and/or Y176-K182 of SEQ ID NO:31. Polynucleotide sequences encoding these polypeptides are also provided. The present invention also encompasses the use of these N-terminal HGPRBMY8 TM5-6 intertransmembrane domain deletion polypeptides as immunogenic and/or antigenic epitopes as described elsewhere herein.

In preferred embodiments, the following C-terminal HGPRBMY8 TM5-6 intertransmembrane domain deletion polypeptides are encompassed by the present invention: C1-K182, C1-A181, C1-A180, C1-K179, C₁-C₁₇₈, C1-Q177, C1-Y176, C₁-C₁₇₅, C1-R174, C1-P173, C1-L172, C1-P171, C1-P170, C1-N169, C1-S168, C1-N167, C1-S166, C1-N165, C1-R164, C1-R163, C1-S162, C1-P161, C1-P160, C1-L159, C1-S158, C1-E157, C1-P156, C1-I155, C1-N154, C1-V153, C1-A152, C1-E151, C1-V150, C1-D149, C1-D148, C1-E147, C1-S146, C1-F145, C1-N144, C1-1143, C1-D142, C1-D141, C1-E140, C1-G139, C1-F138, C1-E137, C1-M136, C1- D135, C1-D134, C1-E133, C1-G132, C1-L131, C1-D130, C1-1129, C1-S128, C₁-C₁₂₇, C1-Q126, C1-N125, C1-V124, C1-E123, C1-T122, C1-R121, C1-G120, C1-K119, C1-D118, C1-A117, C1-K116, C1-M115, C1-S114, C1-N113, C1-E112, C1-E111, C1-V110, C1-K109, C1-T108, C1-S107, C1-G106, C1-E105, C1-K104, C1-G103, C1-E102, C1-M10, C1-S100, C1-G99, C1-D98, C1-S97, C1-A96, C1-V95, C1-T94, C1-S93, C1-S92, C1-E91, C1-R90, C1-V89, C1-E88, C1-E87, C1-S86, C1-G85, C1-A84, C1-E83, C1-V82, C1-S81, C1-S80, C1-E79, C1-S78, C1-T77, C1-G76, C1-T75, C1-S74, C1-G73, C1-E72, C1-K71, C1-A70, C1-K69, C1-L68, C1-S67, C1-G66, C1-D65, C1-K64, C1-A63, C1-E62, C1-M61, C1-R60, C1-G59, C1-E58, C1-K57, C1-A56, C1-K55, C1-V54, C1-E53, C1-G52, C1-E51, C1-H50, C1-Q49, C1-R48, C1-R47, C1-F46, C1-E45, C1-S44, C1-E43, C1-D42, C1-Q41, C1-F40, C1-E39, C1-E38, C1-K37, C1-K36, C1-E35, C1-A34, C1-G33, C1-E32, C1-E31, C1-D30, C1-E29, C1-N28, C1-E27, C1-V26, C1-C25, C1-D24, C1-K23, C1-V22, C1-R21, C1-V20, C1-E19, C1-L18, C1-S17, C1-H16, C1-R15, C1-K14, C1-V13, C1-N12, C1-Y11, C1-L10, C1-L9, C1-A8, and/or C1-H7 of SEQ ID NO:31. Polynucleotide sequences encoding these polypeptides are also provided. The present invention also encompasses the use of these C-terminal HGPRBMY8 TM5-6 intertransmembrane domain deletion polypeptides as immunogenic and/or antigenic epitopes as described elsewhere herein.

In preferred embodiments, the following N-terminal HGPRBMY8 TM6-7 intertransmembrane domain deletion polypeptides are encompassed by the present invention: A1-Q15, V2-Q15, L3-Q15, A4-Q15, V5-Q15, W6-Q15, V7-Q15, D8-Q15, and/or V9-Q15 of SEQ ID NO:32. Polynucleotide sequences encoding these polypeptides are also provided. The present invention also encompasses the use of these N-terminal HGPRBMY8 TM6-7 intertransmembrane domain deletion polypeptides as immunogenic and/or antigenic epitopes as described elsewhere herein.

In preferred embodiments, the following C-terminal HGPRBMY8 TM6-7 intertransmembrane domain deletion polypeptides are encompassed by the present invention: A1-Q15, A1-P14, A1-V13, A1-Q12, A1-T11, A1-E10, A1-V9, A1-D8, and/or A1-V7 of SEQ ID NO:32. Polynucleotide sequences encoding these polypeptides are also provided. The present invention also encompasses the use of these C-terminal HGPRBMY8 TM6-7 intertransmembrane domain deletion polypeptides as immunogenic and/or antigenic epitopes as described elsewhere herein.

The HGPRBMY8 polypeptide was predicted to comprise eight PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: STCTNSTRESNSS (SEQ ID NO:76), QLLQVTNRFIFNL (SEQ ID NO:77), YPSKMTQRRGYLL (SEQ ID NO:78), EAKDGSLKAKEGS (SEQ ID NO:79), EGKEGSTKVEENS (SEQ ID NO:80), KVEENSMKADKGR (SEQ ID NO:81), ESLPPSRRNSNSN (SEQ ID NO:82), and/or GYMHKTIKKEIQD (SEQ ID NO:83). Polynucleotides encoding these polypeptides are also provided. The present invention also encompasses the use of the HGPRBMY8 PKC phosphorylation site polypeptides as immunogenic and/or antigenic epitopes as described elsewhere herein.

The HGPRBMY8 polypeptide was predicted to comprise five casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to aminoacyl-transfer RNA synthetases class-II domains may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: STCTNSTRESNSSH (SEQ ID NO:84), TGTSESSVEARGSE (SEQ ID NO:85), GKEGSTKVEENSMK (SEQ ID NO:86), DDINFSEDDVEAVN (SEQ ID NO:87), and/or PPKEDSHPDLPGTE (SEQ ID NO:88). Polynucleotides encoding these polypeptides are also provided. The present invention also encompasses the use of this casein kinase II phosphorylation site polypeptide as an immunogenic and/or antigenic epitope as described elsewhere herein.

The HGPRBMY8 polypeptide was predicted to comprise two cAMP-and cGMP-dependent protein kinase phosphorylation site using the Motif algorithm (Genetics Computer Group, Inc.). There has been a number of studies relative to the specificity of cAMP-and cGMP-dependent protein kinases. Both types of kinases appear to share a preference for the phosphorylation of serine or threonine residues found close to at least two consecutive N-terminal basic residues.

A consensus pattern for cAMP-and cGMP-dependent protein kinase phosphorylation sites is as follows: [RK](2)-x-[ST], wherein “x” represents any amino acid, and S or T is the phosphorylation site.

Additional information specific to cAMP-and cGMP-dependent protein kinase phosphorylation sites may be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP-and cGMP-dependent protein kinase phosphorylation site polypeptide is encompassed by the present invention: LLYNVKRHSLEVRV (SEQ ID NO:89), and/or SLPPSRRNSNSNPP (SEQ ID NO:90). Polynucleotides encoding this polypeptide are also provided. The present invention also encompasses the use of this cAMP-and cGMP-dependent protein kinase phosphorylation site polypeptide as an immunogenic and/or antigenic epitope as described elsewhere herein.

The HGPRBMY8 polypeptide has been shown to comprise three glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: TSTCTNSTRESNSS (SEQ ID NO:91), STRESNSSHTCMPL (SEQ ID NO:92), and/or GEDDINFSEDDVEA (SEQ ID NO:93). Polynucleotides encoding these polypeptides are also provided. The present invention also encompasses the use of these HGPRBMY8 asparagine glycosylation site polypeptide as immunogenic and/or antigenic epitopes as described elsewhere herein.

The HGPRBMY8 polypeptide was predicted to comprise eight N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N- myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: ISLAHGIIRSTVLVIF (SEQ ID NO:94), CSMIWGASPSYTILSV (SEQ ID NO:95), MEAKDGSLKAKEGSTG (SEQ ID NO:96), LKAKEGSTGTSESSVE (SEQ ID NO:97), KEGSTGTSESSVEARG (SEQ ID NO:98), TVASDGSMEGKEGSTK (SEQ ID NO:99), HPDLPGTEGGTEGKIV (SEQ ID NO:100), and/or LPGTEGGTEGKIVPSY (SEQ ID NO:101). The present invention also encompasses the use of these N-myristoylation site polypeptides as immunogenic and/or antigenic epitopes as described elsewhere herein.

Moreover, in confirmation of HGPRBMY8 representing a novel GPCR, the HGPRBMY8 polypeptide was predicted to comprise a G-protein coupled receptor motif using the Motif algorithm (Genetics Computer Group, Inc.). G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to iF, 2A to 2C, 4, 5A, 5B, 6 and 7, Acetylcholine, muscarinic-type, M1 to M5, Adenosine A1, A2A, A2B and A3, Adrenergic alpha-1A to -1C; alpha-2A to −2D; beta-1 to -3, Angiotensin II types I and II, Bombesin subtypes 3 and 4, Bradykinin B1 and B2, c3a and C5a anaphylatoxin, Cannabinoid CB1 and CB2, Chemokines C—C CC-CKR-1 to CC-CKR-8, Chemokines C—X—C CXC-CKR-1 to CXC-CKR-4, Cholecystokinin-A and cholecystokinin-B/gastrin, Dopamine D1 to D5, Endothelin ET-a and ET-b, fMet-Leu-Phe (FMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-1R), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors CO₆G4.5, C38C10.1, C43C3.2,T27D1.3 and ZC84.4, Three putative receptors encoded in the genome of cytomegalovirus: US27, US28, and UL33., ECRF3, a putative receptor encoded in the genome of herpesvirus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative consensus sequence for GPCRs comprises the conserved triplet and also spans the major part of the third transmembrane helix, and is as follows: [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM], where “X” represents any amino acid.

Additional information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G.L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Garnier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); http://www.gcrdb.uthscsa.edu/: and http://swift.embl-heidelberg.de/7tm/.

In preferred embodiments, the following G-protein coupled receptors signature polypeptide is encompassed by the present invention: SVVSFUVIPLIVMIACYSVVF (SEQ ID NO:102). Polynucleotides encoding this polypeptide are also provided. The present invention also encompasses the use of the HGPRBMY8 G-protein coupled receptors signature polypeptide as immunogenic and/or antigenic epitopes as described elsewhere herein.

For the production of antibodies, various hosts including goats, rabbits, sheep, rats, mice, humans, and others, can be immunized by injection with HGPRBMY8 polypeptide, or any fragment or oligopeptide thereof, which has immunogenic properties. Depending on the host species, various adjuvants may be used to increase the immunological response. Non-limiting examples of suitable adjuvants include Freund's (complete and incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the peptides, fragments, or oligopeptides used to induce antibodies to HGPRBMY8 polypeptide (i.e., immunogens) have an amino acid sequence having at least five amino acids, and more preferably, at least 7-10 amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of HGPRBMY8 amino acids may be fused with those of another protein, such as KLH, and antibodies are produced against the chimeric molecule.

Monoclonal antibodies to HGPRBMY8 polypeptide, or immunogenic fragments thereof, may be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce HGPRBMY8 polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for HGPRBMY8 polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between HGPRBMY8 polypeptide and its specific antibody. A two-site, monoclonal-based immunoassay utilizing monoclonal antibodies reactive with two non-interfering HGPRBMY8 polypeptide epitopes is preferred, but a competitive binding assay may also be employed (Maddox, supra).

Another aspect of the invention relates to a method for inducing an immunological response in a mammal which comprises inoculating the mammal with HGPRBMY8 polypeptide, or a fragment thereof, adequate to produce antibody and/or T cell immune response to protect said animal from infections such as bacterial, fungal, protozoan and viral infections, particularly infections caused by HIV-1 or HIV-2. Yet another aspect of the invention relates to a method of inducing immunological response in a mammal which comprises, delivering HGPRBMY8 polypeptide via a vector directing expression of HGPRBMY8 polynucleotide in vivo in order to induce such an immunological response to produce antibody to protect said animal from diseases.

A further aspect of the invention relates to an immunological/ vaccine formulation (composition) which, when introduced into a mammalian host, induces an immunological response in that mammal to an HGPRBMY8 polypeptide wherein the composition comprises an HGPRBMY8 polypeptide or HGPRBMY8 gene. The vaccine formulation may further comprise a suitable carrier. Since the HGPRBMY8 polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit- dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In an embodiment of the present invention, the polynucleotide encoding the HGPRBMY8 polypeptide, or any fragment or complement thereof, may be used for therapeutic purposes. In one aspect, antisense, to the polynucleotide encoding the HGPRBMY8 polypeptide, may be used in situations in which it would be desirable to block the transcription of the mRNA. In particular, cells may be transformed with sequences complementary to polynucleotides encoding HGPRBMY8 polypeptide. Thus, complementary molecules may be used to modulate HGPRBMY8 polynucleotide and polypeptide activity, or to achieve regulation of gene function. Such technology is now well known in the art, and sense or antisense oligomers or oligonucleotides, or larger fragments, can be designed from various locations along the coding or control regions of polynucleotide sequences encoding HGPRBMY8 polypeptide.

Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors which will express a nucleic acid sequence that is complementary to the nucleic acid sequence encoding the HGPRBMY8 polypeptide. These techniques are described both in J. Sambrook et al., supra and in F. M. Ausubel et al., supra.

Polypeptides used in treatment can also be generated endogenously in the subject, in treatment modalities often referred to as “gene therapy”. Thus for example, cells from a subject may be engineered with a polynucleotide, such as DNA or RNA, to encode a polypeptide ex vivo, and for example, by the use of a retroviral plasmid vector. The cells can then be introduced into the subject.

The genes encoding the HGPRBMY8 polypeptide can be turned off by transforming a cell or tissue with an expression vector that expresses high levels of an HGPRBMY8 polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of the gene encoding the HGPRBMY8 polypeptide, (e.g., signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Ribozymes, i.e., enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding HGPRBMY8 polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes according to the invention may be prepared by any method known in the art for the synthesis of nucleic acid molecules. Such methods include techniques for chemically synthesizing oligonucleotides, for example, solid phase phosphoramidite chemical synthesis. Alternatively, RNA molecules may be generated by in vitro and in vivo transcription of DNA sequences encoding HGPRBMY8. Such DNA sequences may be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules may be modified to increase intracellular stability and half- life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

Many methods for introducing vectors into cells or tissues are available and are equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art.

Any of the therapeutic methods described above may be applied to any individual in need of such therapy, including, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise HGPRBMY8 nucleic acid, polypeptide, or peptides, antibodies to HGPRBMY8 polypeptide, mimetics, agonists, antagonists, or inhibitors of HGPRBMY8 polypeptide or polynucleotide. The compositions may be administered alone, or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the HGPRBMY8 nucleic acid or polypeptide, or functional fragments thereof), the pharmaceutical compositions may contain suitable pharmaceutically acceptable carriers or excipients comprising auxiliaries which facilitate processing of the active compounds into preparations which can be used pharmaceutically. Further details on techniques for formulation and administration are provided in the latest edition of Remington's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of HGPRBMY8 product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, HGPRBMY8 polypeptide, or fragments thereof, antibodies to HGPRBMY8 polypeptide, agonists, antagonists or inhibitors of HGPRBMY8 polypeptide, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The practitioner, who will consider the factors related to the individual requiring treatment, will determine the exact dosage. Dosage and administration are adjusted to provide sufficient levels of the active moiety or to maintain the desired effect. Factors, which may be taken into account, include the severity of the individual's disease state, general health of the patient, age, weight, and gender of the patient, diet, time and frequency of administration, drug combination(s), reaction sensitivities, and tolerance/ response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

In another embodiment of the present invention, antibodies which specifically bind to the HGPRBMY8 polypeptide may be used for the diagnosis of conditions or diseases characterized by expression (or overexpression) of the HGPRBMY8 polynucleotide or polypeptide, or in assays to monitor patients being treated with the HGPRBMY8 polypeptide, or its agonists, antagonists, or inhibitors. The antibodies useful for diagnostic purposes may be prepared in the same manner as those described above for use in therapeutic methods. Diagnostic assays for the HGPRBMY8 polypeptide include methods, which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules, which are known in the art, may be used, several of which are described above. In particular, a method of detecting a G-protein coupled receptor, homologue, or an antibody-reactive fragment thereof, in a sample, comprising: (a) contacting the sample with an antibody specific for the polypeptide, or an antigenic fragment thereof, under conditions in which an antigen-antibody complex can form between the antibody and the polypeptide or antigenic fragment thereof in the sample; and (b) detecting an antigen-antibody complex formed in step (a), wherein detection of the complex indicates the presence of an antigenic fragment thereof, in the sample.

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). In fact, reporter assays have been successfully used for identifying novel small molecule agonists or antagonists against GPCRs as a class of drug targets (Zlokarnik et al., 1998; George et al., 1997; Boss et al., 1996; Rees et al, 2001). In such reporter assays, a promoter is regulated as a direct consequence of activation of specific signal transduction cascades following agonist binding to a GPCR (Alam & Cook 1990; Selbie & Hill, 1998; Boss et al., 1996; George et al., 1997; Gilman, 1987).

A number of response element-based reporter systems have been developed that enable the study of GPCR function. These include cAMP response element (CRE)-based reporter genes for G alpha i/o, G alpha s- coupled GPCRs, Nuclear Factor Activator of Transcription (NFAT)-based reporters for G alpha q/11 11 or the promiscuous G protein G alpha 15/16-coupled receptors and MAP kinase reporter genes for use in Galpha i/o coupled receptors (Selbie & Hill, 1998; Boss et al., 1996; George et al., 1997; Blahos, et al., 2001; Offermann & Simon, 1995; Gilman, 1987; Rees et al., 2001). Transcriptional response elements that regulate the expression of Beta-Lactamase within a CHO K1 cell line (CHO-NFAT/CRE: Aurora Biosciences TM) (Zlokarnik et al., 1998) have been implemented to characterize the function of the orphan HGPRBMY8 polypeptide of the present invention. The system enables demonstration of constitutive G-protein coupling to endogenous cellular signaling components upon intracellular overexpression of orphan receptors. Overexpression has been shown to represent a physiologically relevant event. For example, it has been shown that overexpression occurs in nature during metastatic carcinomas, wherein defective expression of the monocyte chemotactic protein 1 receptor, CCF2, in macrophages is associated with the incidence of human ovarian carcinoma (Sica, et al.,2000; Salcedo et al., 2000). Indeed, it has been shown that overproduction of the Beta 2 Adrenergic Receptor in transgenic mice leads to constitutive activation of the receptor signaling pathway such that these mice exhibit increased cardiac output (Kypson et al., 1999; Dorn et al., 1999). These are only a few of the many examples demonstrating constitutive activation of GPCRs whereby many of these receptors are likely to be in the active, R*, conformation (J.Wess 1997) (see Example 7).

Several assay protocols including ELISA, RIA, and FACS for measuring HGPRBMY8 polypeptide are known in the art and provide a basis for diagnosing altered or abnormal levels of HGPRBMY8 polypeptide expression. Normal or standard values for HGPRBMY8 polypeptide expression are established by combining body fluids or cell extracts taken from normal mammalian subjects, preferably human, with antibody to the HGPRBMY8 polypeptide under conditions suitable for complex formation. The amount of standard complex formation may be quantified by various methods; photometric means are preferred. Quantities of HGPRBMY8 polypeptide expressed in subject sample, control sample, and disease samples from biopsied tissues are compared with the standard values. Deviation between standard and subject values establishes the parameters for diagnosing disease.

### Microarrays and Screening Assays

In another embodiment of the present invention, oligonucleotides, or longer fragments derived from the HGPRBMY8 polynucleotide sequence described herein may be used as targets in a microarray. The microarray can be used to monitor the expression level of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, the nucleic acid sequence, which encodes the HGPRBMY8 polypeptide, may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C.M. Price, 1993, Blood Rev., 7:127-134 and by B.J. Trask, 1991, Trends Genet., 7:149-154.

Fluorescent In Situ Hybridization (FISH), (as described in I. Verma et al., 1988, Human Chromosomes: A Manual of Basic Techniques Pergamon Press, New York, N.Y.) may be correlated with other physical chromosome mapping techniques and genetic map data. Examples of genetic map data can be found in numerous scientific journals, or at Online Mendelian Inheritance in Man (OMIM). Correlation between the location of the gene encoding the HGPRBMY8 polypeptide on a physical chromosomal map and a specific disease, or predisposition to a specific disease, may help delimit the region of DNA associated with that genetic disease. The nucleotide sequences, particularly that of SEQ ID NO:1, or fragments thereof, according to this invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers, even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 1 lq22-23 (R. A. Gatti et al., 1988, Nature, 336:577-580), any sequences mapping to that area may represent associated or regulatory genes for further investigation. The nucleotide sequence of the present invention may also be used to detect differences in the chromosomal location due to translocation, inversion, and the like, among normal, carrier, or affected individuals.

In another embodiment of the present invention, the HGPRBMY8 polypeptide, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between HGPRBMY8 polypeptide, or portion thereof, and the agent being tested, may be measured utilizing techniques commonly practiced in the art. In particular, a method of screening a library of molecules or compounds with an HGPRBMY8 polynucleotide, or fragment thereof, to identify at least one molecule or compound therein which specifically binds to the G-protein coupled receptor polynucleotide sequence, preferably the HGPRBMY8 polynucleotide sequence, or fragment thereof, comprising: (a) combining the G-protein coupled receptor polynucleotide, or fragment thereof, with a library of molecules or compounds under conditions to allow specific binding; and (b) detecting specific binding, thereby identifying a molecule or compound, which specifically binds to a G-protein coupled receptor-encoding polynucleotide sequence. In a further embodiment, the screening method is a high throughput screening method. Preferably, the library is selected from the group consisting of DNA molecules, RNA molecules, artificial chromosome constructions, PNAs, peptides and proteins. In another preferred embodiment, the candidate small molecules or compounds are a drug or therapeutic.

In yet another embodiment, a method of screening for candidate compounds capable of modulating activity of a G-protein coupled receptor-encoding polypeptide, comprising: (a) contacting a test compound with a cell or tissue expressing the G-protein coupled receptor polypeptide, homologue, or fragment thereof; and (b) selecting as candidate modulating compounds those test compounds that modulate activity of the G-protein coupled receptor polypeptide. Preferably, the candidate compounds are agonists or antagonists of G-protein coupled receptor activity. More preferably, the polypeptide activity is associated with the brain.

Another technique for drug screening, which may be used, provides for high throughput screening of compounds having suitable binding affinity to the protein of interest as described in WO 84/03564 (Venton, et al.). In this method, as applied to the HGPRBMY8 protein, large numbers of different small test compounds are synthesized on a solid substrate, such as plastic pins or some other surface. The test compounds are reacted with the HGPRBMY8 polypeptide, or fragments thereof, and washed. Bound HGPRBMY8 polypeptide is then detected by methods well known in the art. Purified HGPRBMY8 polypeptide can also be coated directly onto plates for use in the aforementioned drug screening techniques. Alternatively, non-neutralizing antibodies can be used to capture the peptide and immobilize it on a solid support.

In a further embodiment of this invention, competitive drug screening assays can be used in which neutralizing antibodies, capable of binding the HGPRBMY8 polypeptide, specifically compete with a test compound for binding to the HGPRBMY8 polypeptide. In this manner, the antibodies can be used to detect the presence of any peptide, which shares one or more antigenic determinants with the HGPRBMY8 polypeptide.

Other screening and small molecule (e.g., drug) detection assays which involve the detection or identification of small molecules or compounds that can bind to a given protein, i.e., the HGPRBMY8 polypeptide, are encompassed by the present invention. Particularly preferred are assays suitable for high throughput screening methodologies. In such binding-based screening or detection assays, a functional assay is not typically required. All that is needed is a target protein, preferably substantially purified, and a library or panel of compounds (e.g., ligands, drugs, small molecules) to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP; Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, HGPRBMY8 polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

### Antibodies

Further polypeptides of the invention relate to antibodies and T-cell antigen receptors (TCR) which immunospecifically bind a polypeptide, polypeptide fragment, or variant of SEQ ID NO:2, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)₂ fragments) which are capable of specifically binding to protein. Fab and F(ab′)₂ fragments lack the Fc fragment of intact antibody, clear more rapidly from the circulation of the animal or plant, and may have less non-specific tissue binding than an intact antibody (Wahl et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6M, 5×10-7 M, 107 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem . 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988); and Current Protocols, Chapter 2; which are hereby incorporated herein by reference in its entirety). In a preferred method, a preparation of the HGPRBMY8 protein is prepared and purified to render it substantially free of natural contaminants. Such a preparation is then introduced into an animal in order to produce polyclonal antisera of greater specific activity. For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., pp. 563-681 (1981); Köhler et al., Eur. J. Immunol. 6:511 (1976); Kohler et al., Eur. J. Immunol. 6:292 (1976), or other methods known to the artisan. Other examples of methods which may be employed for producing monoclonal antibodies includes, but are not limited to, the human B- cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Preferably, the immunizing agent consists of an HGPRBMY8 polypeptide or, more preferably, with a HGPRBMY8 polypeptide- expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT- deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. More preferred are the parent myeloma cell line (SP20) as provided by the ATCC. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoabsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra, and/or according to Wands et al. (Gastroenterology 80:225-232 (1981)). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples described herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJR134:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; Cabilly et al., Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985); U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti- idiotypic antibodies can be used to bind a polypeptide of the invention and/or to bind its ligands/receptors, and thereby block its biological activity.

Such anti-idiotypic antibodies capable of binding to the HGPRBMY8 polypeptide can be produced in a two-step procedure. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

The antibodies of the present invention may be bispecific antibodies. Bispecific antibodies are monoclonal, Preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody- antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light- chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4, 676, 980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO:2.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

More preferably, a clone encoding an antibody of the present invention may be obtained according to the method described in the Example section herein.

### Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem . 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non- essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428(1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO:2 may be fused or conjugated to the above antibody portions to increase the in vivo half life of the polypeptides or for use in immunoassays using methods known in the art. Further, the polypeptides corresponding to SEQ ID NO:2 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide- linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem . 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa- histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti- angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M., et al., Medicina, (Aires), 59(6):753-8, (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins' with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template.” MIPs can be derivatized with the same reagents afforded to antibodies. For example, fluorescent ‘super’ MIPs can be coated onto beads or wells for use in highly sensitive separations or assays, or for use in high throughput screening of proteins.

Moreover, MIPs based upon the structure of the polypeptide(s) of the present invention may be useful in screening for compounds that bind to the polypeptide(s) of the invention. Such a MIP would serve the role of a synthetic “receptor” by minimicking the native architecture of the polypeptide. In fact, the ability of a MIP to serve the role of a synthetic receptor has already been demonstrated for the estrogen receptor (Ye, L., Yu, Y., Mosbach, K, Analyst., 126(6):760-5, (2001); Dickert, F, L., Hayden, O., Halikias, K, P, Analyst., 126(6):766-71, (2001)). A synthetic receptor may either be mimicked in its entirety (e.g., as the entire protein), or mimicked as a series of short peptides corresponding to the protein (Rachkov, A., Minoura, N, Biochim, Biophys, Acta., 1544(1-2):255-66, (2001)). Such a synthetic receptor MIPs may be employed in any one or more of the screening methods described elsewhere herein.

MIPs have also been shown to be useful in “sensing” the presence of its mimicked molecule (Cheng, Z., Wang, E., Yang, X, Biosens, Bioelectron., 16(3):179-85, (2001); Jenkins, A, L., Yin, R., Jensen, J. L, Analyst., 126(6):798-802, (2001); Jenkins, A, L., Yin, R., Jensen, J. L, Analyst., 126(6):798-802, (2001)). For example, a MIP designed using a polypeptide of the present invention may be used in assays designed to identify, and potentially quantitate, the level of said polypeptide in a sample. Such a MIP may be used as a substitute for any component described in the assays, or kits, provided herein (e.g., ELISA, etc.).

A number of methods may be employed to create MIPs to a specific receptor, ligand, polypeptide, peptide, organic molecule. Several preferred methods are described by Esteban et al in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem, Soc., 123(9):2072-3, (2001); and Quaglia, M., Chenon, K., Hall, A, J., De, Lorenzi, E., Sellergren, B, J. Am. Chem, Soc., 123(10):2146-54, (2001); which are hereby incorporated by reference in their entirety herein.

### Uses for Antibodies Directed Against Polypeptides of the Invention

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), ppl47-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

### Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

### Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non- fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic Uses Of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, 1L-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10⁻¹⁵ M, and 10-15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting gene expression of a particular gene, or genes, in a human, mammal, and/or other organism. See, for example, International Publication Number WO 00/05391, published Feb. 3, 2000, to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

### Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue- specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid- carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem . 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor- mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging With Antibodies

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell . Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: (a) administering (for example, parenterally, subcutaneously, or intraperitoneally) to a subject an effective amount of a labeled molecule which specifically binds to the polypeptide of interest; (b) waiting for a time interval following the administering for permitting the labeled molecule to preferentially concentrate at sites in the subject where the polypeptide is expressed (and for unbound labeled molecule to be cleared to background level); (c) determining background level; and (d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface- bound recombinant antigens, and a reporter-labeled anti-human antibody for detecting surface-bound anti-antigen antibody.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: a Laboratory Manual, 2^{nd} Edition, Cold Spring Harbor Laboratory Press, USA, (1989).

### Example 1

### Bioinformatics Analysis

G-protein coupled receptor sequences were used as a probes to search human genomic sequence databases. The search program used was gapped BLAST (S. F. Altschul, et al., Nuc. Acids Res., 25:3389-4302 (1997)). The top genomic exon hits from the BLAST results were searched back against the non-redundant protein and patent sequence databases. From this analysis, exons encoding potential full-length sequence of a novel human GPCR, HGPRBMY8, was identified directly from the genomic sequence. The full-length clone of this GPCR was experimentally obtained by RT-PCR using the sequence from genomic data. The complete protein sequence of HGPRBMY8 was analyzed for potential transmembrane domains. TMPRED program (K. Hofmann and W. Stoffel, Biol. Chem., 347:166 (1993) was used for transmembrane prediction. The program predicted seven transmembrane domains and the predicted domains match with the predicted transmembrane domains of related GPCRs at the sequence level. Based on sequence, structure and known GPCR signature sequences, the orphan protein, HGPRBMY8 of the present invention, is a novel human GPCR.

### Example 2

### Cloning of the Novel Human GPCR HGPRBMY8

HGPRBMY8 was cloned from a human brain cDNA library (Clontech; Palo Alto, Calif.) by PCR amplification of the predicted cDNA sequence using sequence specific oligonucleotides. The 5′ sense oligonucleotide was as follows:

| | |
|---|---|
| 5′-GGCCGAATTCGCAACCTGTCTCACGCCCTCTG | (SEQ ID NO:5) |
| G-3′. | |

The 3′ anti-sense oligonucleotide was as follows:

| | |
|---|---|
| 5′-GGCCGAATTCGGACAGTTCAAGGTTTGCCTT | (SEQ ID NO:6) |
| AGAAC-3′. | |

These oligonucleotides contained EcoRI restriction enzyme sites for subcloning the PCR fragment into the mammalian expression vector, pcDNA6. Samples containing human brain cDNA, the 5 prime sense, and 3 prime anti-sense oligonucleotides were subjected to PCR amplification followed by gel purification of the amplified product. The inserts of cDNA clones that were positive by PCR were sized, and two of the largest clones (˜1.6 Kb) were sequenced using conventional sequencing methods. Purified sample was digested with EcoRI, extracted with phenol:chloroform, and ligated into pcDNA6. The resultant plasmids were subjected to DNA sequencing and the sequences were verified by comparison with the database sample.

### Example 3

### Expression Profiling of Novel Human GPCR, HGPRBMY8

The oligonucleotides used for the expression profiling of HGPRBMY8 are:

| | |
|---|---|
| HGPRBMY8-2s: | |
| 5′-GCAGAGCACTCCTCCACTCT-3′ | (SEQ ID NO:34) |
| HGPRBMY8-2a: | |
| 5′-AGCAGGCAATCATGACAATC-3′ | (SEQ ID NO:35) |

These oligonucleotides were used to measure the steady state levels of mRNA by quantitative PCR. Briefly, first strand cDNA was made from commercially available mRNA (Clontech; Palo Alto, Calif.). The relative amount of cDNA used in each assay (2.5 ng of cDNA per assay ) was determined by performing a parallel experiment using a primer pair for the cyclophilin gene, which is expressed in equal amounts in all tissues. The cyclophilin primer pair detected small variations in the amount of cDNA in each sample, and these data were used for normalization of the data obtained with the primer pair for HGPRBMY8. The PCR data were converted into a relative assessment of the difference in transcript abundance among the tissues tested and the data are presented in FIG. 7. Transcripts corresponding to the orphan GPCR, HGPRBMY8, were found to be highly expressed in brain.

### Example 4

### G-Protein Coupled Receptor PCR Expression Profiling

Based on HGPRBMY8's expression in the brain, further analysis was carried out to determine if there was any additional specificity within sub regions. The same PCR primer pair that was used to identify HGPRBMY8 (also referred to as GPCR 58 and GPCR84) cDNA clones was used to measure the steady state levels of mRNA by quantitative PCR.

| | | |
|---|---|---|
| GPCR84-s | GTTAGCCTCACCCACCTGTT | (SEQ ID NO:36) |
| GPCR84-a | CACAATCCAGGTGCCATAGA | (SEQ ID NO:37) |

Briefly, first strand cDNA was made from commercially available brain subregion mRNA (Clontech) and subjected to real time quantitative PCR using a PE 5700 instrument (Applied Biosystems; Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which gives an indication of the number of different DNA sequences present by determining melting Tm. In the case of the HGPRBMY8 primer pair, only one DNA fragment was detected having a homogeneous melting point. Contributions of contaminating genomic DNA to the assessment of tissue abundance is controlled for by performing the PCR with first strand made with and without reverse transcriptase. In all cases, the contribution of material amplified in the no reverse transcriptase controls was negligible.

More specifically, since HGPRBMY8 is expressed at extremely low levels, each PCR reaction contained the amount of first strand cDNA made from 100 nanograms of poly A+ RNA (2.5 nanograms is the standard amount).

The number of reactions and amount of mix needed was first determined. All of the samples were run in triplicate, so sample tubes needed 3.5 reactions worth of mixture using the following formula as a guide (2×# tissue samples+1 no template control+1 for pipetting error)(3.5).

The reaction mixture consisted of the following components and volumes:

| COMPONENTS | VOL/RXN |
|---|---|
| 2 × SybrGreen Master Mix | 25 microliters |
| water | 23.5 microliters |
| primer mix (10 uM ea.) | 0.5 microliters |
| cDNA (100 ng/uL) | 1 microliter |

The mixture was initially made without cDNA for enough reactions as determined above. The mix (171.5 μl) was then aliquoted into sample tubes. cDNA (3.5 μl) was added to each sample tube, mixed gently, and spun down for collection.

Three 50 μl samples were aliquoted to the optical plate, where the primer and sample were set up for sample analysis. The threshold was set in Log view to intersect linear regions of amplification. The background was set in Linear view to 2-3 cycles before the amplification curve appears. The mean values for RT+ was calculated and normalized to Cyclophilin: dct=sample mean−cyclophilin mean. The ddct was determined by subtracting individual dcts from the highest value of dct in the list. The relative abundance was determined by formula 2\hat{ }dd_{Ct}.

Small variations in the amount of cDNA used in each tube was determined by performing a parallel experiment using a primer pair for a gene expressed in equal amounts in all tissues, cyclophilin. These data were used to normalize the data obtained with the HGPRBMY8 primer pair. The PCR data was converted into a relative assessment of the difference in transcript abundance amongst the tissues tested and the data are presented in bar graph form. Transcripts corresponding to HGPRBMY8 are expressed approximately 825 times greater in the caudate nucleus than in the substantia nigra. Low level expression was detected in the thalamus, amygdala, hippocampus, cerebellum and corpus collosum (see FIG. 8).

### Example 5

### Signal Transduction Assays

The activity of GPCRs or homologues thereof, can be measured using any assay suitable for the measurement of the activity of a G protein-coupled receptor, as commonly known in the art. Signal transduction activity of a G protein-coupled receptor can be monitor by monitoring intracellular Ca²⁺, cAMP, inositol 1,4,5-triphosphate (IP₃), or 1,2-diacylglycerol (DAG). Assays for the measurement of intracellular Ca are described in Sakurai et al. (EP 480 381). Intracellular IP3 can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the media of the host cells expressing GPCRs. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by the phospholipase C yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure the IP₃ concentrations, radioactivity labeled H-inositol is added to the media of host cells expressing GPCRs. The ³H-inositol is taken up by the cells and incorporated into IP₃. The resulting inositol triphosphate is separated from the mono and di-phosphate forms and measured (Sakurai et al., EP 480 381). Alternatively, Amersham provides an inositol 1,4,5-triphosphate assay system. With this system Amersham provides tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc. Natl. Acad. Sci. 67:305-312 (1970). In addition, a kit for assaying levels of cAMP is available from Diagnostic Products Corp. (Los Angeles, Calif.).

### Example 6

### GPCR Activity

Another method for screening compounds which are antagonists, and thus inhibit activation of the receptor polypeptide of the present invention is provided. This involves determining inhibition of binding of labeled ligand, such as dATP, dAMP, or UTP, to cells which have the receptor on the surface thereof, or cell membranes containing the receptor. Such a method further involves transfecting a eukaryotic cell with DNA encoding the GPCR polypeptide such that the cell expresses the receptor on its surface. The cell is then contacted with a potential antagonist in the presence of a labeled form of a ligand, such as dATP, dAMP, or UTP. The ligand can be labeled, e.g., by radioactivity, fluorescence, or any detectable label commonly known in the art. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called a binding assay. Naturally, this same technique can be used to determine agonists.

In a further screening procedure, mammalian cells, for example, but not limited to, CHO, HEK 293, Xenopus Oocytes, RBL-2H3, etc., which are transfected, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

In yet another screening procedure, mammalian cells are transfected to express the receptor of interest, and are also transfected with a reporter gene construct that is coupled to activation of the receptor (for example, but not limited to luciferase or beta-galactosidase behind an appropriate promoter). The cells are contacted with a test substance and the receptor agonist (ligand), such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for antagonists or agonists involves introducing RNA encoding the GPCR polypeptide into cells (or CHO, HEK 293, RBL-2H3, etc.) to transiently or stably express the receptor. The receptor cells are then contacted with the receptor ligand, such as dATP, dAMP, or UTP, and a compound to be screened. Inhibition or activation of the receptor is then determined by detection of a signal, such as, cAMP, calcium, proton, or other ions.

### Example 7

### Functional Characterization of HGPRBMY8

The putative GPCR HGPRBMY8 cDNA was PCR amplified using PFUTM (Stratagene). The primers used in the PCR reaction were specific to the HGPRBMY8 polynucleotide and were ordered from Gibco BRL (5 prime primer: 5′-GTCCCCAAGCTTGCACCATGACGTCCACCTGCACCAACAGCA-3′ (SEQ ID NO:38). The following 3 prime primer was used to add a Flag-tag epitope to the HGPRBMY8 polypeptide for immunocytochemistry: 5′-CGGGATCCTACTTGTCGTCGTCGTCCTTGTAGTCCATAGGAAAAGTAGCAG AATCGTAGGAA-3′ (SEQ ID NO:39). The product from the PCR reaction was isolated from a 0.8% Agarose gel (Invitrogen) and purified using a Gel Extraction Kit™ from Qiagen.

The purified product was then digested overnight along with the pcDNA3.1 Hygro™ mammalian expression vector from Invitrogen using the HindIII and BamHI restriction enzymes (New England Biolabs). These digested products were then purified using the Gel Extraction Kit TM from Qiagen and subsequently ligated to the pcDNA3.1 Hygro™ expression vector using a DNA molar ratio of 4 parts insert: 1 vector. All DNA modification enzymes were purchased from NEB. The ligation was incubated overnight at 16 degrees Celsius, after which time, one microliter of the mix was used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones were isolated using the Wizard DNA Miniprep System™ from Promega. Positive clones were then confirmed and scaled up for purification using the Qiagen Maxiprep™ plasmid DNA purification kit.

### Cell Line Generation

The pcDNA3. Ihygro vector containing the orphan HGPRBMY8 cDNA were used to transfect CHO-NFAT/CRE (Aurora Biosciences) cells using Lipofectamine 2000™ according to the manufacturers specifications (Gibco BRL). Two days later, the cells were split 1:3 into selective media (DMEM 11056, 600 μg/ml Hygromycin, 200 μg/ml Zeocin, 10% FBS). All cell culture reagents were purchased from Gibco BRL-Invitrogen.

The CHO-NFAT/CRE and the CHO-NFAT G alpha 15 cell lines, transiently or stably transfected with the orphan HGPRBMY8 GPCR, were analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the orphan HGPRBMY8 GPCR, is examined by analyzing the fluorescence emission of the transformed cells at 447 nm and 518 nm. The changes in gene expression can be visualized using Beta-Lactamase as a reporter, that, when induced by the appropriate signaling cascade, hydrolyzes an intracellularly loaded, membrane-permeant ester substrate Cephalosporin-Coumarin-Fluorescein2/Acetoxymethyl (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme is readily apparent since each enzyme molecule produced is capable of changing the fluorescence of many CCF2/AM™ substrate molecules. A schematic of this cell based system is shown below.

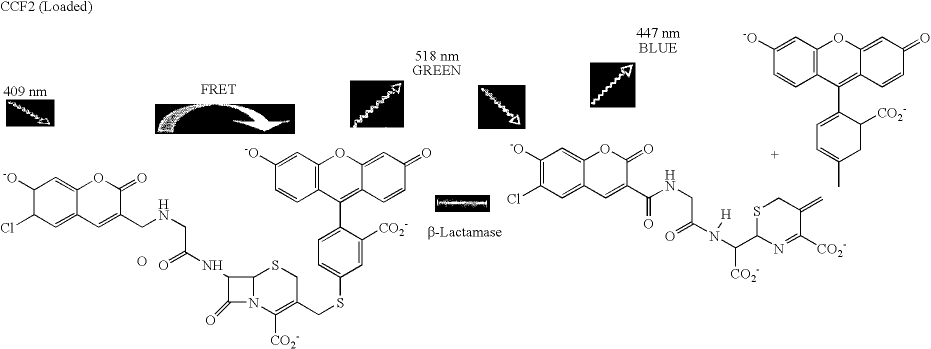

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only—thus giving rise to blue fluorescent emission at 447 nm.

Fluorescent emissions were detected using a Nikon-TE300 microscope equipped with an excitation filter (D405/10X-25), dichroic reflector (430DCLP), and a barrier filter for dual DAPI/FITC (510 nM) to visually capture changes in Beta-Lactamase expression. The FACS Vantage SE is equiped with a Coherent Enterprise II Argon Laser and a Coherent 302C Krypton laser. In flow cytometry, UV excitation at 351-364 nm from the Argon Laser or violet excitation at 407 nm from the Krypton laser are used. The optical filters on the FACS Vantage SE are HQ460/50m and HQ535/40m bandpass separated by a 490 dichroic mirror.

Prior to analyzing the fluorescent emissions from the cell lines as described above, the cells were loaded with the CCF2/AM substrate. A 6×CCF2/AM loading buffer was prepared whereby 1 mM CCF2/AM (Aurora Biosciences) was dissolved in 100% DMSO (Sigma). Stock solution (12 μl) was added to 60 μl of 100 mg/ml Pluronic F127 (Sigma) in DMSO containing 0.1% Acetic Acid (Sigma). This solution was added while vortexing to 1 mL of Sort Buffer (PBS minus calcium and magnesium-Gibco-25 mM HEPES-Gibco-pH 7.4, 0.1% BSA). Cells were placed in serum-free media and the 6×CCF2/AM was added to a final concentration of 1×. The cells were then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998; and BD Biosciences,1999.

### Immunocytochemistry

The cell lines transfected and selected for expression of Flag-epitope tagged orphan GPCRs were analyzed by immunocytochemistry. The cells were plated at 1×10³ in each well of a glass slide (VWR). The cells were rinsed with PBS followed by acid fixation for 30 minutes at room temperature using a mixture of 5% Glacial Acetic Acid/90% ethanol. The cells were then blocked in 2% BSA and 0.1% Triton in PBS, incubated for 2 h at room temperature or overnight at 4° C. A monoclonal FITC antibody directed against FLAG was diluted at 1:50 in blocking solution and incubated with the cells for 2 h at room temperature. Cells were then washed three times with 0.1% Triton in PBS for five minutes. The slides were overlayed with mounting media dropwise with Biomedia-Gel Mount™ (Biomedia; Containing Anti-Quenching Agent). Cells were examined at 10× magnification using the Nikon TE300 equiped with FITC filter (535 nm).

There is strong evidence that certain GPCRs exhibit a cDNA concentration-dependent constitutive activity through cAMP response element (CRE) luciferase reporters (Chen et al., 1999). In an effort to demonstrate functional coupling of HGPRBMY8 to known GPCR second messenger pathways, the HGPRBMY8 polypeptide was expressed at high constitutive levels in the CHO-NFAT/CRE cell line. To this end, the HGPRBMY8 cDNA was PCR amplified and subcloned into the pcDNA3.1 hygro™ mammalian expression vector as described herein. Early passage CHO-NFAT/CRE cells were then transfected with the resulting pcDNA3.1 hygro TM/HGPRBMY8 construct. Transfected and non-transfected CHO-NFAT/CRE cells (control) were loaded with the CCF2 substrate and stimulated with 10 nM PMA, 1 μM Thapsigargin (NFAT stimulator), and 10 μM Forskolin (CRE stimulator) to fully activate the NFAT/CRE element. The cells were then analyzed for fluorescent emission by FACS.

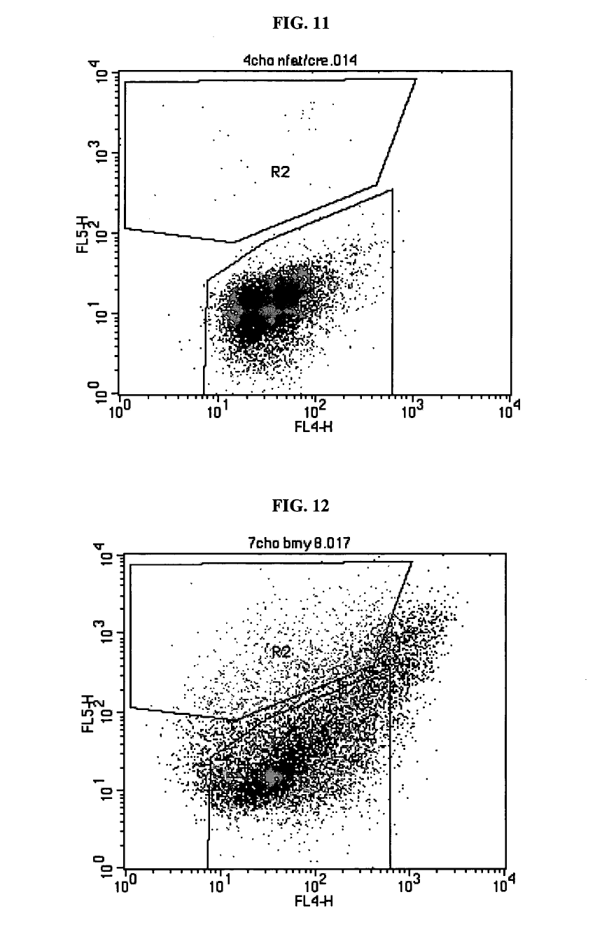

The FACS profile demonstrates the constitutive activity of HGPRBMY8 in the CHO-NFAT/CRE line as evidenced by the significant population of cells with blue fluorescent emission at 447 nm (see FIG. 12: Blue Cells). FIG. 12 further describes CHO-NFAT/CRE cell lines transfected with the pcDNA3.1 Hygro™/HGPRBMY8 mammalian expression vector. The cells were analyzed via FACS according to their wavelength emission at 518 nM (Channel R3-Green Cells), and 447 nM (Channel R2-Blue Cells). As shown, overexpression of HGPRBMY8 results in functional coupling and subsequent activation of beta lactamase gene expression, as evidenced by the significant number of cells with fluorescent emission at 447 nM relative to the non-transfected control CHO-NFAT/CRE cells (shown in FIG. 11). As expected, the NFAT/CRE response element in the untransfected control cell line was not activated (i.e., beta lactamase not induced), enabling the CCF2 substrate to remain intact, and resulting in the green fluorescent emission at 518 nM (see FIG. 11-Green Cells). FIG. 11 describes control CHO-NFAT/CRE (Nuclear Factor Activator of Transcription (NFAT)/cAMP response element (CRE)) cell lines, in the absence of the pcDNA3.1 Hygro™/HGPRBMY8 mammalian expression vector transfection. The cells were analyzed via FACS (Fluorescent Assisted Cell Sorter) according to their wavelength emission at 518 nM (Channel R3-Green Cells), and 447 nM (Channel R2-Blue Cells). As shown, the vast majority of cells emit at 518 nM, with minimal emission observed at 447 nM. The latter is expected since the NFAT/CRE response elements remain dormant in the absence of an activated G-protein dependent signal transduction pathway (e.g., pathways mediated by Gq/111 or Gs coupled receptors). As a result, the cell permeant, CCF2/AM™ (Aurora Biosciences; Zlokarnik, et al., 1998) substrate remains intact and emits light at 518 nM.

A very low level of leaky Beta Lactamase expression was detectable as evidenced by the small population of cells emitting at 447 nm. Analysis of a stable pool of cells transfected with HGPRBMY8 revealed constitutive coupling of the cell population to the NFAT/CRE response element, activation of Beta Lactamase and cleavage of the substrate (FIG. 12-Blue Cells). These results demonstrate that overexpression of HGPRBMY8 leads to constitutive coupling of signaling pathways known to be mediated by Gq/11 or G alpha 15/16 or Gs coupled receptors that converge to activate either the NFAT or CRE response elements respectively (Boss et al., 1996; Chen et al., 1999).

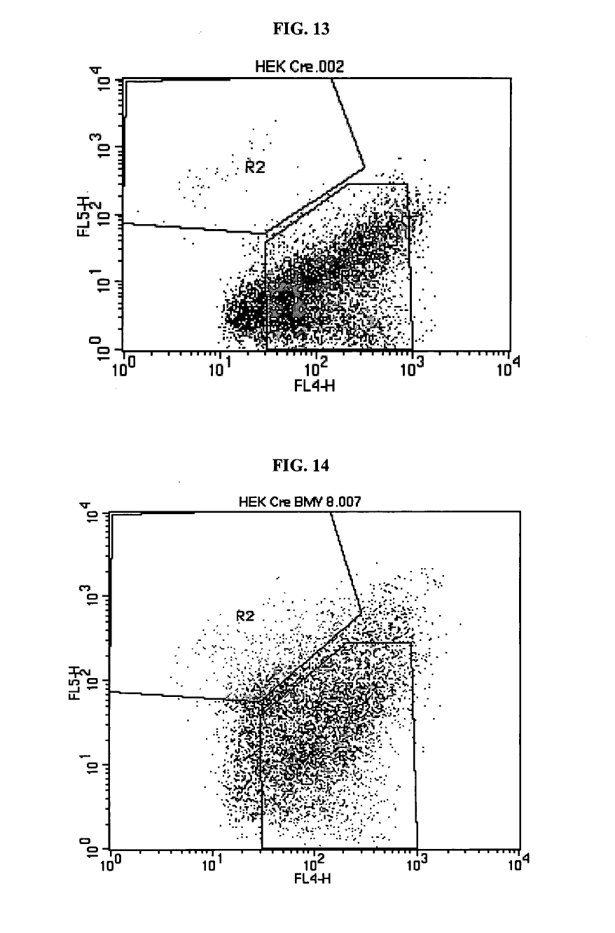

In an effort to further characterize the observed functional coupling of the HGPRBMY8 polypeptide, its ability to couple to the cAMP response element (CRE) independent of the NFAT response element was examined. To this end, HEK-CRE cell line that contained only the integrated 3XCRE linked to the Beta-Lactamase reporter was transfected with the pcDNA3.1 hygro™/HGPRBMY8 construct. Analysis of the fluorescence emission from this stable pool showed that HGPRBMY8 constitutively coupled to the cAMP mediated second messenger pathways (see FIG. 14 relative to FIG. 13). FIG. 14 describes FACS analysis of HEK-CRE cell lines transfected with the pcDNA3.1 Hygro™/HGPRBMY8 mammalian expression vector according to their wavelength emission at 518 nM (Channel R3-Green Cells), and 447 nM (Channel R2-Blue Cells). As shown, overexpression of HGPRBMY8 in the HEK-CRE cells resulted in functional coupling, as evidenced by the insignificant background level of cells with fluorescent emission at 447 nM. FIG. 13 describes HEK-CRE cell lines in the absence of the pcDNA3.1 Hygro™/HGPRBMY8 mammalian expression vector transfection. The cells were analyzed via FACS (Fluorescent Assisted Cell Sorter) according to their wavelength emission at 518 nM (Channel R3-Green Cells), and 447 nM (Channel R2-Blue Cells). As shown, the vast majority of cells emit at 518 nM, with minimal emission observed at 447 nM. The latter is expected since the CRE response elements remain dormant in the absence of an activated G-protein dependent signal transduction pathway (e.g., pathways mediated by Gs coupled receptors). As a result, the cell permeant, CCF2/AM™ (Aurora Biosciences; Zlokarnik, et al., 1998) substrate remains intact and emits light at 518 nM.

Experiments have shown that known G coupled receptors do demonstrate constitutive activation when overexpressed in the HEK-CRE cell line. For example, direct activation of adenylate cyclase using 10 μM Forskolin has been shown to activate CRE and the subsequent induction of Beta-Lactamase in the HEK-CRE cell line (data not shown). In conclusion, the results are consistent with HGPRBMY8 representing a functional GPCR analogous to known Gs coupled receptors (Boss et al., 1996).

### Demonstration of Cellular Expression

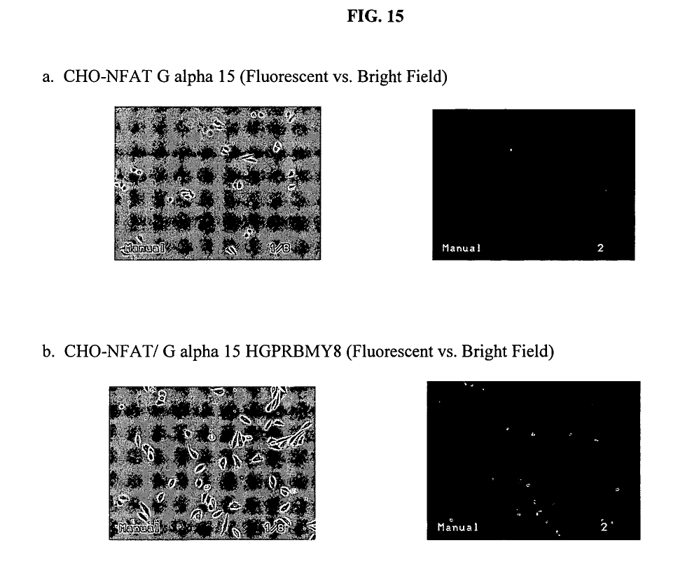

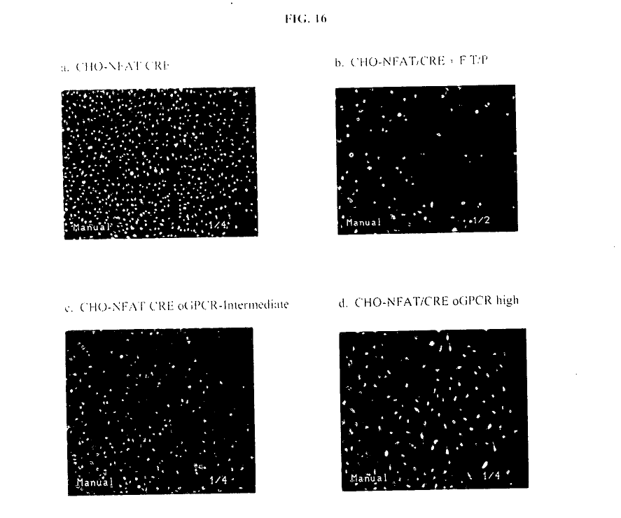

HGPRBMY8 was tagged at the C-terminus using the Flag epitope and inserted into the pcDNA3. 1 hygro™ expression vector, as described herein. Immunocytochemistry of CHO-NFAT/CRE cell lines transfected with the Flag-tagged HGPRBMY8 construct with FITC conjugated Anti Flag monoclonal antibody demonstrated that HGPRBMY8 is indeed a cell surface receptor. The immunocytochemistry also confirmed expression of the HGPRBMY8 in the CHO-NFAT/CRE cell lines. Briefly, CHO-NFAT/CRE cell lines were transfected with pcDNA3.1 hygro Tm/HGPRBMY8-Flag vector, fixed with 70% methanol, and permeablized with 0.1% TritonX100. The cells were then blocked with 1% Serum and incubated with a FITC conjugated Anti Flag monoclonal antibody at 1:50 dilution in PBS-Triton. The cells were then washed several times with PBS-Triton, overlayed with mounting solution, and fluorescent images were captured (see FIGS. 15A-D). FIG. 15 describes CHO-NFAT/CRE cell lines transfected with the pcDNA3.1 Hygro™/HGPRBMY8-FLAG mammalian expression vector subjected to immunocytochemistry using an FITC conjugated Anti Flag monoclonal antibody. Panel A shows the transfected CHO-NFAT/CRE cells under visual wavelengths, and panel B shows the fluorescent emission of the same cells at 530 nm after illumination with a mercury light source. The cell expression is clearly evident in panel B, and is consistent with the HGPRBMY8 polypeptide representing a member of the GPCR family. The control cell line, non-transfected CHO-NFAT /CREcell line, exhibited no detectable background fluorescence (FIG. 15). The HGPRBMY8-FLAG tagged expressing CHO-NFAT /CRE line exhibited specific plasma membrane expression as indicated (FIG. 15). These data provide clear evidence that HGPRBMY8 is expressed in these cells and the majority of the protein is localized to the cell surface. Cell surface localization in consistent with HGPRBM8 representing a 7 transmembrane domain containing GPCR. Taken together, the data indicate that HGPRBMY8 is a cell surface GPCR that can function through increases in either cAMP or Ca²⁺ signal transduction pathways via G alpha 15.

### Screening Paradigm

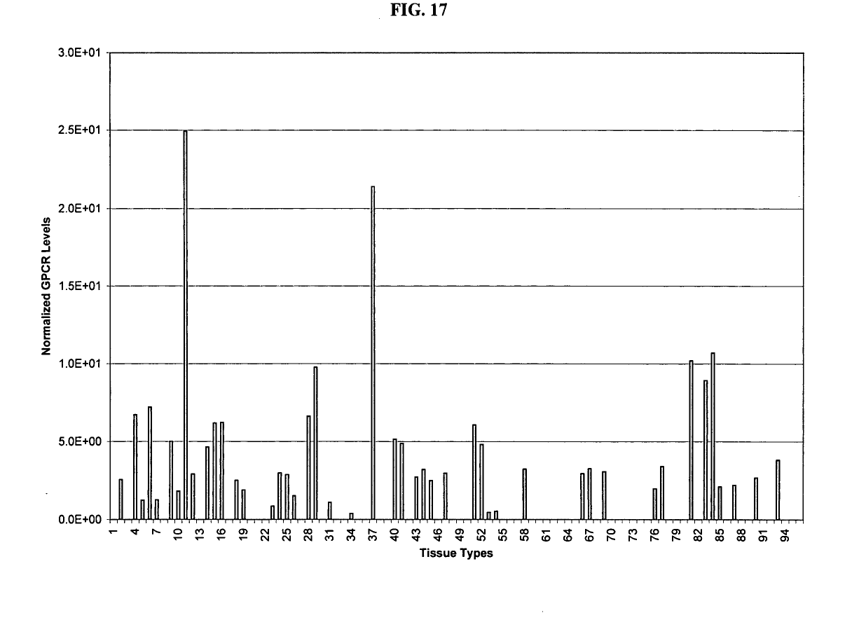

The Aurora Beta-Lactamase technology provides a clear path for identifying agonists and antagonists of the HGPRBMY8 polypeptide. Cell lines that exhibit a range of constitutive coupling activity have been identified by sorting through HGPRBMY8 transfected cell lines using the FACS Vantage SE (see FIG. 16). For example, cell lines have been sorted that have an intermediate level of orphan GPCR expression, which also correlates with an intermediate coupling response, using the LJL analyst. Such cell lines will provide the opportunity to screen, indirectly, for both agonists and antagonists of HGPRBMY8 by looking for inhibitors that block the beta lactamase response, or agonists that increase the beta lactamase response. As described herein, modulating the expression level of beta lactamase directly correlates with the level of cleaved CCF2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca²⁺]i. The data shown below represent cell lines that have been engineered with the desired pattern of HGPRBMY8 expression to enable the identification of potent small molecule agonists and antagonists. HGPRBMY8 modulator screens may be carried out using a variety of high throughput methods known in the art, though preferably using the fully automated Aurora UHTSS system. The untransfected CHO-NFAT/CRE cell line represents the relative background level of beta lactamase expression (FIG. 16; panel a). FIG. 16 describes several CHO-NFAT/CRE cell lines transfected with the pcDNA3.1 Hygro™/HGPRBMY8 mammalian expression vector isolated via FACS that had either intermediate or high beta lactamase expression levels of constitutive activation. Panel A shows untransfected CHO-NFAT/CRE cells prior to stimulation with 10 nM PMA, 1 μM Thapsigargin, and 10 μM Forskolin (−P/T/F). Panel B shows CHO-NFAT/CRE cells after stimulation with 10 nM PMA, 1 μM Thapsigargin, and 10 μM Forskolin (+P/T/F). Panel C shows a representative orphan GPCR (oGPCR) transfected CHO-NFAT/CRE cells that have an intermediate level of beta lactamase expression. Panel D shows a representative orphan GPCR transfected CHO-NFAT/CRE that have a high level of beta lactamase expression. Following treatment with a cocktail of 10 nM PMA, 1 μM Thapsigargin, and 10 μM Forskolin (FIG. 16; P/T/F; panel b), the cells fully activate the CRE-NFAT response element demonstrating the dynamic range of the assay. Panel C (FIG. 16) represents an orphan transfected CHO-NFAT/CRE cell line that shows an intermediate level of beta lactamase expression post P/T/F stimulation, while panel D (FIG. 16) represents a orphan transfected CHO-NFAT/CRE cell line that shows a high level of constitutive beta lactamase expression.

### Example 8

### G-Protein Coupled Receptor PCR Expression Profiling

RNA quantification was performed using the Taqman real-time-PCR fluorogenic assay. The Taqman assay is one of the most precise methods for assaying the concentration of nucleic acid templates .

All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL; Rockville, Md.). Eighty percent confluent cells were washed twice with phosphate-buffered saline (GibcoBRL) and harvested using 0.25% trypsin (GibcoBRL). RNA was prepared using the RNeasy Maxi Kit from Qiagen (Valencia, Calif.).

cDNA template for real-time PCR was generated using the Superscript First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75×SYBR Green I (Sigma); 1×SYBR Green PCR Buffer (SOmMTris-HCl pH8.3, 75 mM KCl); 10% DMSO; 3 mM MgCl₂; 300 M each dATP, dGTP, dTTP, dCTP; 1 U Platinum Taq DNA Polymerase High Fidelity (Cat# 11304-029; Life Technologies; Rockville, Md.); 1:50 dilution; ROX (Life Technologies). Real-time PCR was performed using an Applied Biosystems 5700 Sequence Detection System. Conditions were 95C for 10 min (denaturation and activation of Platinum Taq DNA Polymerase), 40 cycles of PCR (95C for 15 sec, 60C for 1 min). PCR products are analyzed for uniform melting using an analysis algorithm built into the 5700 Sequence Detection System.

| | |
|---|---|
| Forward primer: 745 GPCR84-2s: | |
| 5′-GCAGAGCACTCCTCCACTCT-3′; | (SEQ ID NO:34) |
| and | |
| Reverse primer: 746 GPCR84-2a: | |
| 5′-AGCAGGCAATCATGACAATC-3′. | (SEQ ID NO:35) |

cDNA quantification used in the normalization of template quantity was performed using Taqman technology. Taqman reactions are prepared as follows: The reaction mix consisted of 20 ng first strand cDNA; 25 nM GAPDH-F3, Forward Primer; 250 nM GAPDH-R1 Reverse Primer; 200 nM GAPDH-PVIC Taqman Probe (fluorescent dye labeled oligonucleotide primer); 1× Buffer A (Applied Biosystems); 5.5 mM MgCl2; 300 M dATP, dGTP, dTTP, dCTP; 1 U Amplitaq Gold (Applied Biosystems). GAPDH, D-glyceraldehyde -3-phosphate dehydrogenase, was used as control to normalize mRNA levels.

Real-time PCR was performed using an Applied Biosystems 7700 Sequence Detection System. Conditions were 95C for 10 min. (denaturation and activation of Amplitaq Gold), 40 cycles of PCR (95C for 15 sec, 60C for 1 min).

The sequences for the GAPDH oligonucleotides used in the Taqman reactions are as follows:

| | |
|---|---|
| GAPDH-F3 - 5′-AGCCGAGCCACATCGCT-3′ | (SEQ ID NO:60) |
| GAPDH-R1 - 5′-GTGACCAGGCGCCCAATAC-3′ | (SEQ ID NO:61) |
| GAPDH-PVIC Taqman ® Probe-VIC - 5′- | (SEQ ID NO:62) |
| CAAATCCGTTGACTCCGACCTTCACCTT-3′ | |
| TAMRA. | |

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the Ct equation (Applied Biosystems Prism 7700 Sequence Detection System User Bulletin #2), which is used to calculate a GAPDH normalized relative cDNA level for each specific cDNA. The Ct equation is as follows: relative quantity of nucleic acid template=2^{Ct}=2^{(Cta-Ctb)}, where Cta=Ct target−Ct GAPDH, and Ctb=Ct reference−Ct GAPDH. (No reference cell was used for the calculation of relative quantity; Ctb was defined as 21).

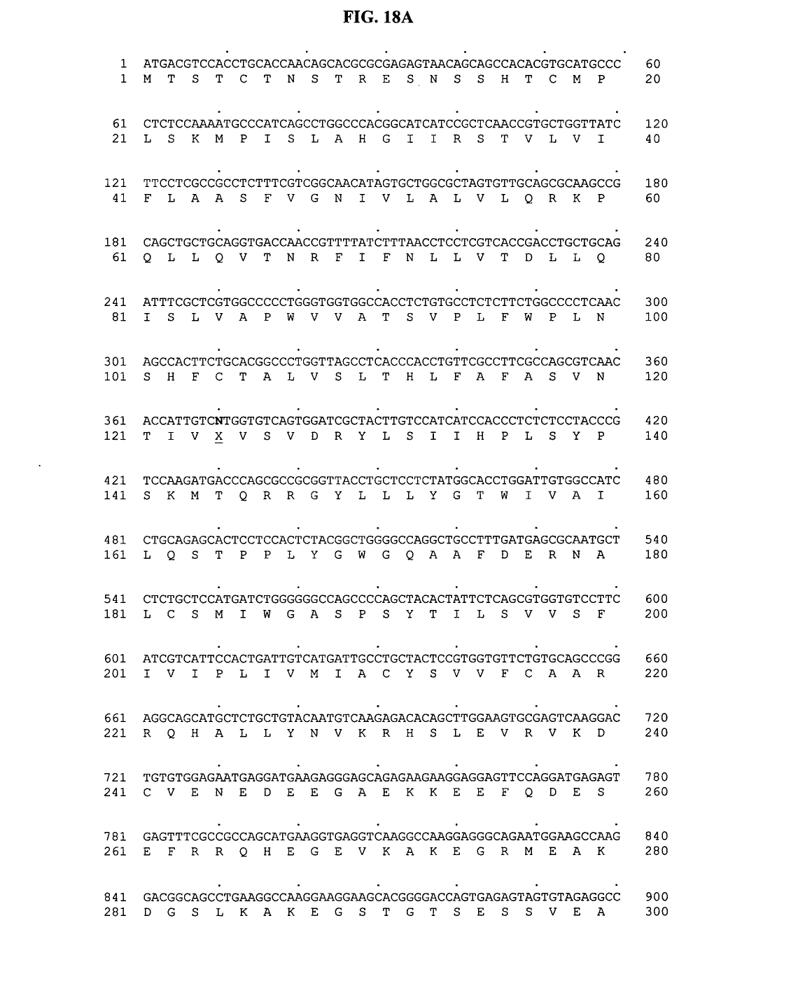

The Graph # of Table 1 corresponds to the tissue type position number of FIG. 17. HGPRBMY8 (also known as GPCR84 or GPCR58) was found to have relatively low expression in the tumor cell lines assayed in the OCLP-1 (oncology cell line panel). HGPRBMY8 message appears to be especially scarce in breast tumor cell lines. The average HGPRBMY8 message level in lung tumor cell lines is 2-3 fold lower than the average for other cell lines assayed.

TABLE 1

| Graph # | Name | Tissue | Ct GAPDH | Ct GPCR84 | dCt | ddCt | Quant. |
|---|---|---|---|---|---|---|---|
| 1 | AIN 4 | breast | 17.49 | 40 | 22.51 | 1.51 | 0.0E+00 |
| 2 | AIN 4T | breast | 17.15 | 36.8 | 19.65 | −1.35 | 2.5E+00 |
| 3 | AIN4/myc | breast | 17.81 | 40 | 22.19 | 1.19 | 0.0E+00 |
| 4 | BT-20 | breast | 17.9 | 36.15 | 18.25 | −2.75 | 6.7E+00 |
| 5 | BT-474 | breast | 17.65 | 38.34 | 20.69 | −0.31 | 1.2E+00 |
| 6 | BT-483 | breast | 17.45 | 35.6 | 18.15 | −2.85 | 7.2E+00 |
| 7 | BT-549 | breast | 17.55 | 38.21 | 20.66 | −0.34 | 1.3E+00 |
| 8 | DU4475 | breast | 18.1 | 40 | 21.9 | 0.9 | 0.0E+00 |
| 9 | H3396 | breast | 18.04 | 36.71 | 18.67 | −2.33 | 5.0E+00 |
| 10 | HBL100 | breast | 17.02 | 37.16 | 20.14 | −0.86 | 1.8E+00 |
| 11 | Her2 MCF-7 | breast | 19.26 | 35.62 | 16.36 | −4.64 | 2.5E+01 |
| 12 | HS 578T | breast | 17.83 | 37.28 | 19.45 | −1.55 | 2.9E+00 |
| 13 | MCF7 | breast | 17.83 | 40 | 22.17 | 1.17 | 0.0E+00 |
| 14 | MCF-7/AdrR | breast | 17.23 | 36.01 | 18.78 | −2.22 | 4.7E+00 |
| 15 | MDAH 2774 | breast | 16.87 | 35.24 | 18.37 | −2.63 | 6.2E+00 |
| 16 | MDA-MB-175-VII | breast | 15.72 | 34.08 | 18.36 | −2.64 | 6.2E+00 |
| 17 | MDA-MB-231 | breast | 17.62 | 40 | 22.38 | 1.38 | 0.0E+00 |
| 18 | MDA-MB-453 | breast | 17.9 | 37.57 | 19.67 | −1.33 | 2.5E+00 |
| 19 | MDA-MB-468 | breast | 17.49 | 37.58 | 20.09 | −0.91 | 1.9E+00 |
| 20 | Pat-21 R60 | breast | 35.59 | 40 | 4.41 | −16.59 | ND |
| 21 | SKBR3 | breast | 17.12 | 40 | 22.88 | 1.88 | 0.0E+00 |
| 22 | T47D | breast | 18.86 | 40 | 21.14 | 0.14 | 0.0E+00 |
| 23 | UACC-812 | breast | 17.06 | 38.26 | 21.2 | 0.2 | 8.7E−01 |
| 24 | ZR-75-1 | breast | 15.95 | 35.36 | 19.41 | −1.59 | 3.0E+00 |
| 25 | C-33A | cervical | 17.49 | 36.96 | 19.47 | −1.53 | 2.9E+00 |
| 26 | Ca Ski | cervical | 17.38 | 37.78 | 20.4 | −0.6 | 1.5E+00 |
| 27 | HeLa | cervical | 17.59 | 40 | 22.41 | 1.41 | 0.0E+00 |
| 28 | HT-3 | cervical | 17.42 | 35.69 | 18.27 | −2.73 | 6.6E+00 |
| 29 | ME-180 | cervical | 16.86 | 34.57 | 17.71 | −3.29 | 9.8E+00 |
| 30 | SiHa | cervical | 18.07 | 40 | 21.93 | 0.93 | 0.0E+00 |
| 31 | SW756 | cervical | 15.59 | 36.45 | 20.86 | −0.14 | 1.1E+00 |
| 32 | CACO-2 | colon | 17.56 | 40 | 22.44 | 1.44 | 0.0E+00 |
| 33 | CCD-112Co | colon | 18.03 | 40 | 21.97 | 0.97 | 0.0E+00 |
| 34 | CCD-33Co | colon | 17.07 | 39.44 | 22.37 | 1.37 | 3.9E−01 |
| 35 | Colo 205 | colon | 18.02 | 40 | 21.98 | 0.98 | 0.0E+00 |
| 36 | Colo 320DM | colon | 17.01 | 40 | 22.99 | 1.99 | 0.0E+00 |
| 37 | Colo201 | colon | 17.89 | 34.47 | 16.58 | −4.42 | 2.1E+01 |
| 38 | Cx-1 | colon | 18.79 | 40 | 21.21 | 0.21 | 0.0E+00 |
| 39 | ddH2O | colon | 40 | 40 | 0 | −21 | ND |
| 40 | HCT116 | colon | 17.59 | 36.22 | 18.63 | −2.37 | 5.2E+00 |
| 41 | HCT116/epo5 | colon | 17.71 | 36.42 | 18.71 | −2.29 | 4.9E+00 |
| 42 | HCT116/ras | colon | 17.18 | 40 | 22.82 | 1.82 | 0.0E+00 |
| 43 | HCT116/TX15CR | colon | 17.36 | 36.91 | 19.55 | −1.45 | 2.7E+00 |
| 44 | HCT116/vivo | colon | 17.7 | 37.01 | 19.31 | −1.69 | 3.2E+00 |
| 45 | HCT116/VM46 | colon | 17.87 | 37.55 | 19.68 | −1.32 | 2.5E+00 |
| 46 | HCT116/VP35 | colon | 17.3 | 40 | 22.7 | 1.7 | 0.0E+00 |
| 47 | HCT-8 | colon | 17.44 | 36.86 | 19.42 | −1.58 | 3.0E+00 |
| 48 | HT-29 | colon | 17.9 | 40 | 22.1 | 1.1 | 0.0E+00 |
| 49 | LoVo | colon | 17.64 | 40 | 22.36 | 1.36 | 0.0E+00 |
| 50 | LS 174T | colon | 17.93 | 40 | 22.07 | 1.07 | 0.0E+00 |
| 51 | LS123 | colon | 17.65 | 36.05 | 18.4 | −2.6 | 6.1E+00 |
| 52 | MIP | colon | 16.92 | 35.65 | 18.73 | −2.27 | 4.8E+00 |
| 53 | SK-CO-1 | colon | 17.75 | 39.84 | 22.09 | 1.09 | 4.7E−01 |
| 54 | SW1417 | colon | 17.22 | 39.11 | 21.89 | 0.89 | 5.4E−01 |
| 55 | SW403 | colon | 18.39 | 40 | 21.61 | 0.61 | 0.0E+00 |
| 56 | SW480 | colon | 17 | 40 | 23 | 2 | 0.0E+00 |
| 57 | SW620 | colon | 17.16 | 40 | 22.84 | 1.84 | 0.0E+00 |
| 58 | SW837 | colon | 18.35 | 37.65 | 19.3 | −1.7 | 3.2E+00 |
| 59 | T84 | colon | 16.44 | 40 | 23.56 | 2.56 | 0.0E+00 |
| 60 | CCD-18Co | colon, fibroblast | 17.19 | 40 | 22.81 | 1.81 | 0.0E+00 |
| 61 | HT-1080 | fibrosarcoma | 17.16 | 40 | 22.84 | 1.84 | 0.0E+00 |
| 62 | CCRF-CEM | leukemia | 17.07 | 40 | 22.93 | 1.93 | 0.0E+00 |
| 63 | HL-60 | leukemia | 17.54 | 40 | 22.46 | 1.46 | 0.0E+00 |
| 64 | K562 | leukemia | 18.42 | 40 | 21.58 | 0.58 | 0.0E+00 |
| 65 | A-427 | lung | 18 | 40 | 22 | 1 | 0.0E+00 |
| 66 | A549 | lung | 17.63 | 37.06 | 19.43 | −1.57 | 3.0E+00 |
| 67 | Calu-3 | lung | 18.09 | 37.38 | 19.29 | −1.71 | 3.3E+00 |
| 68 | Calu-6 | lung | 16.62 | 40 | 23.38 | 2.38 | 0.0E+00 |
| 69 | ChaGo-K-1 | lung | 17.79 | 37.16 | 19.37 | −1.63 | 3.1E+00 |
| 70 | DMS 114 | lung | 18.14 | 40 | 21.86 | 0.86 | 0.0E+00 |
| 71 | LX-1 | lung | 18.17 | 40 | 21.83 | 0.83 | 0.0E+00 |
| 72 | MRC-5 | lung | 17.3 | 40 | 22.7 | 1.7 | 0.0E+00 |
| 73 | MSTO-211H | lung | 16.81 | 40 | 23.19 | 2.19 | 0.0E+00 |
| 74 | NCI-H596 | lung | 17.73 | 40 | 22.27 | 1.27 | 0.0E+00 |
| 75 | SHP-77 | lung | 18.66 | 40 | 21.34 | 0.34 | 0.0E+00 |
| 76 | Sk-LU-1 | lung | 15.81 | 35.83 | 20.02 | −0.98 | 2.0E+00 |
| 77 | SK-MES-1 | lung | 17.1 | 36.33 | 19.23 | −1.77 | 3.4E+00 |
| 78 | SW1271 | lung | 16.45 | 40 | 23.55 | 2.55 | 0.0E+00 |
| 79 | SW1573 | lung | 17.14 | 40 | 22.86 | 1.86 | 0.0E+00 |
| 80 | SW900 | lung | 18.17 | 40 | 21.83 | 0.83 | 0.0E+00 |
| 81 | Hs 294T | melanoma | 17.73 | 35.38 | 17.65 | −3.35 | 1.0E+01 |
| 82 | A2780/DDP-R | ovarian | 21.51 | 40 | 18.49 | −2.51 | 0.0E+00 |
| 83 | A2780/DDP-S | ovarian | 17.89 | 35.73 | 17.84 | −3.16 | 8.9E+00 |
| 84 | A2780/epo5 | ovarian | 17.54 | 35.12 | 17.58 | −3.42 | 1.1E+01 |
| 85 | A2780/TAX-R | ovarian | 18.4 | 38.33 | 19.93 | −1.07 | 2.1E+00 |
| 86 | A2780/TAX-S | ovarian | 17.83 | 40 | 22.17 | 1.17 | 0.0E+00 |
| 87 | Caov-3 | ovarian | 15.5 | 35.35 | 19.85 | −1.15 | 2.2E+00 |
| 88 | ES-2 | ovarian | 17.22 | 40 | 22.78 | 1.78 | 0.0E+00 |
| 89 | HOC-76 | ovarian | 34.3 | 40 | 5.7 | −15.3 | ND |
| 90 | OVCAR-3 | ovarian | 17.09 | 36.66 | 19.57 | −1.43 | 2.7E+00 |
| 91 | PA-1 | ovarian | 17.33 | 40 | 22.67 | 1.67 | 0.0E+00 |
| 92 | SW 626 | ovarian | 16.94 | 40 | 23.06 | 2.06 | 0.0E+00 |
| 93 | UPN251 | ovarian | 17.69 | 36.75 | 19.06 | −1.94 | 3.8E+00 |
| 94 | LNCAP | prostate | 18.17 | 40 | 21.83 | 0.83 | 0.0E+00 |
| 95 | PC-3 | prostate | 17.25 | 40 | 22.75 | 1.75 | 0.0E+00 |
| 96 | A431 | squamous | 19.85 | 40 | 20.15 | −0.85 | 0.0E+00 |

### Example 9

### Phage Display Methods for Identifying Peptide Ligands or Modulators of Orphan GPCRS

### Library Construction

Two HGPRBMY libraries were used for identifying peptides that may function as modulators. Specifically, a 15-mer library was used to identify peptides that may function as agonists or antagonists. The 15-mer library is an aliquot of the 15-mer library originally constructed by G. P. Smith (Scott, J K and Smith, G P. 1990, Science 249:386-390). A 40-mer library was used for identifying natural ligands and constructed essentially as previously described, using an M13 phage library displaying random 38-amino acid peptides as a source of novel sequences with affinity to selected targets (B K Kay, et al. 1993, Gene 128:59-65). This method for constructing the 40-mer library was followed with the exception that a 15 base pair complementary region was used to anneal the two oligonucleotides, as opposed to 6, 9, or 12 base pairs, as described below.

The oligos used are: Oligo 1: 5′-CGAAGCGTAAGGGCCCAGCCGGCCNN (BNN×19) BCCGGGTCCGGGCGGC-3′ (SEQ ID NO:63) and Oligo2: 5′-AAAAGGAAAAAAGCGGCCGC (VNN×20) GCCGCCCGGACCCGG-3′ (SEQ ID NO:64), where N=A+G+C+T and B=C+G+T and V=C+A+G.

The oligos were are annealed via their 15 base pair complimentary sequences which encode a constant ProGlyProGlyGly (SEQ ID NO:65) pentapeptide sequence between the random 20 amino acid segments, and then extended by standard procedure using Klenow enzyme. This was followed by endonuclease digestion using Sfi1 and Not1 enzymes and ligation to Sfi1 and Not1 cleaved pCantab5E (Pharmacia). The ligation mixture was electroporated into E. coli XL1Blue and phage clones were essentially generated as suggested by the manufacturer (Pharmacia) for making ScFv antibody libraries in pCantab5E.

### Sequencing Bound Phage

Standard procedures commonly known in the art were used. Phage in eluates were infected into E. coli host strain (TG1 for the 15-mer library; XL1Blue for the 40-mer library) and plated for single colonies. Colonies were grown in liquid and sequenced by standard procedure which involved: 1) generating PCR product with suitable primers of the library segments in the phage genome (15-mer library) or pCantab5E (40-mer library); and 2) sequencing PCR products using one primer of each PCR primer pair. Sequences were visually inspected or were inspected by using the Vector NTI alignment tool.

### Peptide Modulators Of The Present Invention

The following serve as non-limiting examples of HGPRBMY8 peptide modulators:

| | |
|---|---|
| GDFWYEACESSCAFW | (SEQ ID NO:66) |
| LEWGSDVFYDVYDCC | (SEQ ID NO:67) |
| CLRSGTGCAFQLYRF | (SEQ ID NO:68) |
| NNFPCLRSGRNCDAG | (SEQ ID NO:69) |
| RIVPNGYFNVHGRSL | (SEQ ID NO:70) |
| RIDSCAKYFLRSCD | (SEQ ID NO:71) |

### Peptide Synthesis

Peptides were synthesized on Fmoc-Knorr amide resin [N-(9-fluorenyl)methoxycarbonyl-Knorr amide-resin, Midwest Biotech, Fishers, Ind.] with an Applied Biosystems (Foster City, Calif.) model 433A synthesizer and the FastMoc chemistry protocol (0.25 mmol scale) supplied with the instrument. Amino acids were double coupled as their N-alpha-Fmoc- derivatives and reactive side chains were protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group was removed by the multi-step treatment with piperidine in N-Methylpyrrolidone as described by the manufacturer. The N-terminal free amines were then treated with 10% acetic anhydride, 5% Diisopropylamine in N-Methylpyrrolidone to yield the N-acetyl-derivative. The protected peptidyl-resins were simultaneously deprotected and removed from the resin by standard methods. The lyophilized peptides were purified on C₁₈ to apparent homogeneity as judged by RP-HPLC analysis. Predicted peptide molecular weights were verified by electrospray mass spectrometry (J. Biol. Chem. vol. 273, pp.12041-12046, 1998).

Cyclic analogs were prepared from the crude linear products. The cysteine disulfide was formed using one of the following methods:

### Method 1

A sample of the crude peptide was dissolved in water at a concentration of 0.5 mg/mL and the pH adjusted to 8.5 with NH₄OH. The reaction was stirred at room temperature, and monitored by RP-HPLC. Once complete, the reaction was brought to pH 4 with acetic acid and lyophilized. The product was purified and characterized as above.

### Method 2

A sample of the crude peptide was dissolved at a concentration of 0.5 mg/mL in 5% acetic acid. The pH was adjusted to 6.0 with NH₄OH. DMSO (20% by volume) was added and the reaction was stirred overnight. After analytical RP-HPLC analysis, the reaction was diluted with water and triple lyophilized to remove DMSO. The crude product was purified by preparative RP-HPLC. (JACS. vol. 113, 6657, 1991).

### Assessing Affect of Peptides on GPCR Function

The effect of any one of these peptides on the function of the GPCR of the present invention may be determined by adding an effective amount of each peptide to each functional assay. Representative functional assays are described more specifically herein, particularly Example 7.

### Uses Of The Peptide Modulators Of The Present Invention

The aforementioned peptides of the present invention are useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention may be useful as HGPRBMY8 agonists. Alternatively, the peptide modulators of the present invention may be useful as HGPRBMY8 antagonists of the present invention. In addition, the peptide modulators of the present invention may be useful as competitive inhibitors of the HGPRBMY8 cognate ligand(s), or may be useful as non-competitive inhibitors of the HGPRBMY8 cognate ligand(s).

Furthermore, the peptide modulators of the present invention may be useful in assays designed to either deorphan the HGPRBMY8 polypeptide of the present invention, or to identify other agonists or antagonists of the HGPRBMY8 polypeptide of the present invention, particularly small molecule modulators.

### Example 10

### Method of Creating N- and C-Terminal Deletion Mutants Corresponding to the HGPRBMY8 Polypeptide

As described elsewhere herein, the present invention encompasses the creation of N- and C-terminal deletion mutants, in addition to any combination of N- and C-terminal deletions thereof, corresponding to the HGPRBMY8 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length HGPRBMY8 polypeptide sequence, appropriate primers of about 15-25 nucleotides derived from the desired 5′ and 3′ positions of SEQ ID NO:1 may be designed to PCR amplify, and subsequently clone, the intended N- and/or C-terminal deletion mutant. Such primers could comprise, for example, an initiation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, Kozac sequences, or other sequences discussed and/or referenced herein.

For example, in the case of the T36 to P508 N-terminal deletion mutant, the following primers could be used to amplify a cDNA fragment corresponding to this deletion mutant:

| | | |
|---|---|---|
| 5′ Primer | 5′-GCAGCA GCGGCCGC ACCGTGCTGGTTATCTTCCTCGCCG-3′ | (SEQ ID NO:72) |
| | NotI | |
| 3′ Primer | 5′-GCAGCA GTCGAC AGGAAAAGTAGCAGAATCGTAGG-3′ | (SEQ ID NO:73) |
| | SalI | |

For example, in the case of the M1 to Y454 C-terminal deletion mutant, the following primers could be used to amplify a cDNA fragment corresponding to this deletion mutant:

| | | |
|---|---|---|
| 5′ Primer | 5′-GCAGCA GCGGCCGC ATGACGTCCACCTGCACCAACAGC -3′ | (SEQ ID NO:74) |
| | NotI | |
| 3′ Primer | 5′-GCAGCA GTCGAC ATAGACATAGGGGTGGATGCAGCAC -3′ | (SEQ ID NO:75) |
| | SalI | |

Representative PCR amplification conditions are provided below, although the skilled artisan would appreciate that other conditions may be required for efficient amplification. A 100 μl PCR reaction mixture may be prepared using long of the template DNA (cDNA clone of HGPRBMY8), 200 μM 4dNTPs, 1 μM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HGPRBMY8 gene (SEQ ID NO:1), and ‘X’ is equal to the most N-terminal amino acid of the intended N-terminal deletion mutant. The first term provides the start 5′ nucleotide position of the 5′ primer, while the second term provides the end 3′ nucleotide position of the 5′ primer corresponding to sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HGPRBMY8 gene (SEQ ID NO:1), and ‘X’ is equal to the most C-terminal amino acid of the intended N-terminal deletion mutant. The first term provides the start 5′ nucleotide position of the 3′ primer, while the second term provides the end 3′ nucleotide position of the 3′ primer corresponding to the anti-sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N- terminal and C-terminal deletion mutant of the present invention. The skilled artisan would appreciate that modifications of the above nucleotide positions may be necessary for optimizing PCR amplification.

In preferred embodiments, the following N-terminal HGPRBMY8 deletion polypeptides are encompassed by the present invention: M1-P508, T2-P508, S3-P508, T4-P508, C5-P508, T6-P508, N7-P508, S8-P508, T9-P508, R10-P508, E11-P508, S12-P508, N13-P508, S14-P508, S15-P508, H16-P508, T17-P508, C18-P508, M19-P508, P20-P508, L21-P508, S22-P508, K23-P508, M24-P508, P25-P508, 126-P508, S27-P508, L28-P508, A29-P508, H30-P508, G31-P508, 132-P508, 133-P508, R34-P508, S35-P508, T36-P508, V37-P508, L38-P508, V39-P508, 140-P508, F41-P508, L42-P508, A43-P508, A44-P508, S45-P508, F46-P508, V47-P508, G48-P508, N49-P508, 150-P508, V51-P508, L52-P508, A53-P508, L54-P508, V55-P508, L56-P508, Q57-P508, R58-P508, K59-P508, P60-P508, Q61-P508, L62-P508, L63-P508, Q64-P508, V65-P508, T66-P508, N67-P508, R68-P508, F69-P508, 170-P508, F71-P508, N72-P508, L73-P508, L74-P508, V75-P508, T76-P508, D77-P508, L78-P508, L79-P508, Q80-P508, 181-P508, S82-P508, L83-P508, V84-P508, A85-P508, P86-P508, W87-P508, V88-P508, V89-P508, A90-P508, T91-P508, S92-P508, V93-P508, P94-P508, L95-P508, F96-P508, W97-P508, P98-P508, L99-P508, N100-P508, S101-P508, H102-P508, F103-P508, C104-P508, T105-P508, A106-P508, L107-P508, V108-P508, S109-P508, L110-P508, T111-P508, H112-P508, L113-P508, F114-P508, A115-P508, F116-P508, A117-P508, S118-P508, V119-P508, N120-P508, T121-P508, 1122-P508, V123-P508, L124-P508, V125-P508, S126-P508, V127-P508, D128-P508, R129-P508, Y130-P508, L131-P508, S132-P508, 1133-P508, 1134-P508, H135-P508, P136-P508, L137-P508, S138-P508, Y139-P508, P140-P508, S141-P508, K142-P508, M143-P508, T144-P508, Q145-P508, R146-P508, R147-P508, G148-P508, Y149-P508, L150-P508, L151-P508, L152-P508, Y153-P508, G154-P508, T155-P508, W156-P508, 1157-P508, V158-P508, A159-P508, 1160-P508, L161-P508, Q162-P508, S163-P508, T164-P508, P165-P508, P166-P508, L167-P508, Y168-P508, G169-P508, W170-P508, G171-P508, Q172-P508, A173-P508, A174-P508, F175-P508, D176-P508, E177-P508, R178-P508, N179-P508, A180-P508, L181-P508, C182-P508, S183-P508, M184-P508, 1185-P508, W186-P508, G187-P508, A188-P508, S189-P508, P190-P508, S191-P508, Y192-P508, T193-P508, I194-P508, L195-P508, S196-P508, V197-P508, V198-P508, S199-P508, F200-P508, 1201-P508, V202-P508, 1203-P508, P204-P508, L205-P508, 1206-P508, V207-P508, M208-P508, 1209-P508, A210-P508, C211-P508, Y212-P508, S213-P508, V214-P508, V215-P508, F216-P508, C217-P508, A218-P508, A219-P508, R220-P508, R221-P508, Q222-P508, H223-P508, A224-P508, L225-P508, L226-P508, Y227-P508, N228-P508, V229-P508, K230-P508, R231-P508, H232-P508, S233-P508, L234-P508, E235-P508, V236-P508, R237-P508, V238-P508, K239-P508, D240-P508, C241-P508, V242-P508, E243-P508, N244-P508, E245-P508, D246-P508, E247-P508, E248-P508, G249-P508, A250-P508, E251-P508, K252-P508, K253-P508, E254-P508, E255-P508, F256-P508, Q257-P508, D258-P508, E259-P508, S260-P508, E261-P508, F262-P508, R263-P508, R264-P508, Q265-P508, H266-P508, E267-P508, G268-P508, E269-P508, V270-P508, K271-P508, A272-P508, K273-P508, E274-P508, G275-P508, R276-P508, M277-P508, E278-P508, A279-P508, K280-P508, D281-P508, G282-P508, S283-P508, L284-P508, K285-P508, A286-P508, K287-P508, E288-P508, G289-P508, S290-P508, T291-P508, G292-P508, T293-P508, S294-P508, E295-P508, S296-P508, S297-P508, V298-P508, E299-P508, A300-P508, R301-P508, G302-P508, S303-P508, E304-P508, E305-P508, V306-P508, R307-P508, E308-P508, S309-P508, S310-P508, T311-P508, V312-P508, A313-P508, S314-P508, D315-P508, G316-P508, S317-P508, M318-P508, E319-P508, G320-P508, K321-P508, E322-P508, G323-P508, S324-P508, T325-P508, K326-P508, V327-P508, E328-P508, E329-P508, N330-P508, S331-P508, M332-P508, K333-P508, A334-P508, D335-P508, K336-P508, G337-P508, R338-P508, T339-P508, E340-P508, V341-P508, N342-P508, Q343-P508, C344-P508, S345-P508, 1346-P508, D347-P508, L348-P508, G349-P508, E350-P508, D351-P508, D352-P508, M353-P508, E354-P508, F355-P508, G356-P508, E357-P508, D358-P508, D359-P508, 1360-P508, N361-P508, F362-P508, S363-P508, E364-P508, D365-P508, D366-P508, V367-P508, E368-P508, A369-P508, V370-P508, N371-P508, 1372-P508, P373-P508, E374-P508, S375-P508, L376-P508, P377-P508, P378-P508, S379-P508, R380-P508, R381-P508, N382-P508, S383-P508, N384-P508, S385-P508, N386-P508, P387-P508, P388-P508, L389-P508, P390-P508, R391-P508, C392-P508, Y393-P508, Q394-P508, C395-P508, K396-P508, A397-P508, A398-P508, K399-P508, V400-P508, 1401-P508, F402-P508, 1403-P508, 1404-P508, 1405-P508, F406-P508, S407-P508, Y408-P508, V409-P508, L410-P508, S411-P508, L412-P508, G413-P508, P414-P508, Y415-P508, C416-P508, F417-P508, L418-P508, A419-P508, V420-P508, L421-P508, A422-P508, V423-P508, W424-P508, V425-P508, D426-P508, V427-P508, E428-P508, T429-P508, Q430-P508, V431-P508, P432-P508, Q433-P508, W434-P508, V435-P508, 1436-P508, T437-P508, 1438-P508, 1439-P508, 1440-P508, W441-P508, L442-P508, F443-P508, F444-P508, L445-P508, Q446-P508, C447-P508, C448-P508, 1449-P508, H450-P508, P451-P508, Y452-P508, V453-P508, Y454-P508, G455-P508, Y456-P508, M457-P508, H458-P508, K459-P508, T460-P508, 1461-P508, K462-P508, K463-P508, E464-P508, 1465-P508, Q466-P508, D467-P508, M468-P508, L469-P508, K470-P508, K471-P508, F472-P508, F473-P508, C474-P508, K475-P508, E476-P508, K477-P508, P478-P508, P479-P508, K480-P508, E481-P508, D482-P508, S483-P508, H484-P508, P485-P508, D486-P508, L487-P508, P488-P508, G489-P508, T490-P508, E491-P508, G492-P508, G493-P508, T494-P508, E495-P508, G496-P508, K497-P508, 1498-P508, V499-P508, P500-P508, S501-P508, and/or Y502-P508 of SEQ ID NO:2. Polynucleotide sequences encoding these polypeptides are also provided. The present invention also encompasses the use of these N-terminal HGPRBMY8 deletion polypeptides as immunogenic and/or antigenic epitopes as described elsewhere herein.

In preferred embodiments, the following C-terminal HGPRBMY8 deletion polypeptides are encompassed by the present invention: M1-P508, M1-F507, M1-T506, M1-A505, M1-S504, M1-D503, M1-Y502, M1-S501, M1-P500, M1-V499, M1-1498, M1-K497, M1-G496, M1-E495, M1-T494, M1-G493, M1-G492, M1-E491, M1-T490, M1-G489, M1-P488, M1-L487, M1-D486, M1-P485, M1-H484, M1-S483, M1-D482, M1-E481, M1-K480, M1-P479, M1-P478, M1-K477, M1-E476, M1-K475, M1-C474, M1-F473, M1-F472, M1-K471, M1-K470, M1-L469, M1-M468, M1-D467, M1-Q466, M1-1465, M1-E464, M1-K463, M1-K462, M1-1461, M1-T460, M1-K459, M1-H458, M1-M457, M1-Y456, M1-G455, M1-Y454, M1-V453, M1-Y452, M1-P451, M1-H450, M1-1449, M1-C448, M1-C447, M1-Q446, M1-L445, M1-F444, M1-F443, M1-L442, M1-W441, M1-1440, M1-1439, M1-1438, M1-T437, M1-1436, M1-V435, M1-W434, M1-Q433, M1-P432, M1-V431, M1-Q430, M1-T429, M1-E428, M1-V427, M1-D426, M1-V425, M1-W424, M1-V423, M1-A422, M1-L421, M1-V420, M1-A419, M1-L418, M1-F417, M1-C416, M1-Y415, M1-P414, M1-G413, M1-L412, M1-S411, M1-L410, M1-V409, M1-Y408, M1-S407, M1-F406, M1-1405, M1-1404, M1-1403, M1-F402, M1-1401, M1-V400, M1-K399, M1-A398, M1-A397, M1-K396, M1-C395, M1-Q394, M1-Y393, M1-C392, M1-R391, M1-P390, M1-L389, M1-P388, M1-P387, M1-N386, M1-S385, M1-N384, M1-S383, M1-N382, M1-R381, M1-R380, M1-S379, M1-P378, M1-P377, M1-L376, M1-S375, M1-E374, M1-P373, M1-1372, M1-N371, M1-V370, M1-A369, M1-E368, M1-V367, M1-D366, M1-D365, M1-E364, M1-S363, M1-F362, M1-N361, M1-1360, M1-D359, M1-D358, M1-E357, M1-G356, M1-F355, M1-E354, M1-M353, M1-D352, M1-D351, M1-E350, M1-G349, M1-L348, M1-D347, M1-1346, M1-S345, M1-C344, M1-Q343, M1-N342, M1-V341, M1-E340, M1-T339, M1-R338, M1-G337, M1-K336, M1-D335, M1-A334, M1-K333, M1-M332, M1-S331, M1-N330, M1-E329, M1-E328, M1-V327, M1-K326, M1-T325, M1-S324, M1-G323, M1-E322, M1-K321, M1-G320, M1-E319, M1-M318, M1-S317, M1-G316, M1-D315, M1-S314, M1-A313, M1-V312, M1-T311, M1-S310, M1-S309, M1-E308, M1-R307, M1-V306, M1-E305, M1-E304, M1-S303, M1-G302, M1-R301, M1-A300, M1-E299, M1-V298, M1-S297, M1-S296, M1-E295, M1-S294, M1-T293, M1-G292, M1-T291, M1-S290, M1-G289, M1-E288, M1-K287, M1-A286, M1-K285, M1-L284, M1-S283, M1-G282, M1-D281, M1-K280, M1-A279, M1-E278, M1-M277, M1-R276, M1-G275, M1-E274, M1-K273, M1-A272, M1-K271, M1-V270, M1-E269, M1-G268, M1-E267, M1-H266, M1-Q265, M1-R264, M1-R263, M1-F262, M1-E261, M1-S260, M1-E259, M1-D258, M1-Q257, M1-F256, M1-E255, M1-E254, M1-K253, M1-K252, M1-E251, M1-A250, M1-G249, M1-E248, M1-E247, M1-D246, M1-E245, M1-N244, M1-E243, M1-V242, M1-C241, M1-D240, M1-K239, M1-V238, M1-R237, M1-V236, M1-E235, M1-L234, M1-S233, M1-H232, M1-R231, M1-K230, M1-V229, M1-N228, M1-Y227, M1-L226, M1-L225, M1-A224, M1-H223, M1-Q222, M1-R221, M1-R220, M1-A219, M1-A218, M1-C217, M1-F216, M1-V215, M1-V214, M1-S213, M1-Y212, M1-C211, M1-A210, M1-1209, M1-M208, M1-V207, M1-1206, M1-L205, M1-P204, M1-1203, M1-V202, M1-1201, M1-F200, M1-S199, M1-V198, M1-V197, M1-S196, M1-L195, M1-I194, M1-T193, M1-Y192, M1-S191, M1-P190, M1-S189, M1-A188, M1-G187, M1-W186, M1-1185, M1-M184, M1-S183, M1-C182, M1-L181, M1-A180, M1-N179, M1-R178, M1-E177, M1-D176, M1-F175, M1-A174, M1-A173, M1-Q172, M1-G171, M1-W170, M1-G169, M1-Y168, M1-L167, M1-P166, M1-P165, M1-T164, M1-S163, M1-Q162, M1-L161, M1-I160, M1-A159, M1-V158, M1-1157, M1-W156, M1-T155, M1-G154, M1-Y153, M1-L152, M1-L151, M1-L150, M1-Y149, M1-G148, M1-R147, M1-R146, M1-Q145, M1-T144, M1-M143, M1-K142, M1-S141, M1-P140, M1-Y139, M1-S138, M1-L137, M1-P136, M1-H135, M1-1134, M1-1133, M1-S132, M1-L131, M1-Y130, M1-R129, M1-D128, M1-V127, M1-S126, M1-V125, M1-L124, M1-V123, M1-1122, M1-T121, M1-N120, M1-V119, M1-S118, M1-A117, M1-F116, M1-A115, M1-F114, M1-L113, M1-H112, M1-T111, M1-L110, M1-S109, M1-V108, M1-L107, M1-A106, M1-T105, M1-C104, M1-F103, M1-H102, M1-S100, M1-N100, M1-L99, M1-P98, M1-W97, M1-F96, M1-L95, M1-P94, M1-V93, M1-S92, M1-T91, M1-A90, M1-V89, M1-V88, M1-W87, M1-P86, M1-A85, M1-V84, M1-L83, M1-S82, M1-181, M1-Q80, M1-L79, M1-L78, M1-D77, M1-T76, M1-V75, M1-L74, M1-L73, M1-N72, M1-F71, M1-170, M1-F69, M1-R68, M1-N67, M1-T66, M1-V65, M1-Q64, M1-L63, M1-L62, M1-Q61, M1-P60, M1-K59, M1-R58, M1-Q57, M1-L56, M1-V55, M1-L54, M1-A53, M1-L52, M1-V51, M1-150, M1-N49, M1-G48, M1-V47, M1-F46, M1-S45, M1-A44, M1-A43, M1-L42, M1-F41, M1-140, M1-V39, M1-L38, M1-V37, M1-T36, M1-S35, M1-R34, M1-133, M1-132, M1-G31, M1-H30, M1-A29, M1-L28, M1-S27, M1-126, M1-P25, M1-M24, M1-K23, M1-S22, M1-L21, M1-P20, M1-M19, M1-C18, M1-T17, M1-H16, M1-S15, M1-S14, M1-N13, M1-S12, M1-E11, M1-R10, M1-T9, M1-S8, and/or M1-N7 of SEQ ID NO:2. Polynucleotide sequences encoding these polypeptides are also provided. The present invention also encompasses the use of these C-terminal HGPRBMY8 deletion polypeptides as immunogenic and/or antigenic epitopes as described elsewhere herein.

Alternatively, preferred polypeptides of the present invention may comprise polypeptide sequences corresponding to, for example, internal regions of the HGPRBMY8 polypeptide (e.g., any combination of both N- and C-terminal HGPRBMY8 polypeptide deletions) of SEQ ID NO:2. For example, internal regions could be defined by the equation: amino acid NX to amino acid CX, wherein NX refers to any N-terminal deletion polypeptide amino acid of HGPRBMY8 (SEQ ID NO:2), and where CX refers to any C-terminal deletion polypeptide amino acid of HGPRBMY8 (SEQ ID NO:2). Polynucleotides encoding these polypeptides are also provided. The present invention also encompasses the use of these polypeptides as an immunogenic and/or antigenic epitope as described elsewhere herein.

### Example 11

### Method of Enhancing the Biological Activity/Functional Characteristics of Invention Through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest - regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments -further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly, the DNA substrate to be subjected to the DNA shuffling reaction is prepared. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4% g of the DNA substrate(s) would be digested with 0.0015 units of Dnase I (Sigma) per microliter in 100 μl of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/μl. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for Smin using an MJ Research (Cambridge, Mass.) PTC-150 thermocycler. After the assembly reaction is completed, a 1:40 dilution of the resulting primeness product would then be introduced into a PCR mixture (using the same buffer mixture used for the assembly reaction) containing 0.8 um of each primer and subjecting this mixture to 15 cycles of PCR (using 94 C for 30s, 50 C for 30s, and 72 C for 30s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6): 1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F.R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16,000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 12

### Method of Discovering Additional Single Nucleotide Polymorphisms (SNPS) of the Present Invention

Additional SNPs may be discovered in the polynucleotides of the present invention based on comparative DNA sequencing of PCR products derived from genomic DNA from multiple individuals. The genomic DNA samples may be purchased from Coriell Institute (Collingswood, N.J.). PCR amplicons may be designed to cover the entire coding region of the exons using the Primer3 program (Rozen S 2000). Exon-intron structure of candidate genes and intron sequences may be obtained by blastn search of Genbank cDNA sequences against the human genome draft sequences. The sizes of these PCR amplicons will vary according to the exon- intron structure. All the samples may be amplified from genomic DNA (20 ng) in reactions (50 μl) containing 10 mM Tris-Cl pH 8.3, 50 mM KCl, 2.5 mM MgCl₂, 150 uM dNTPs, 3 uM PCR primers, and 3.75 U TaqGold DNA polymerase (PE Biosystems).

PCR is performed in MJ Research Tetrad machines under a cycling condition of 94 degrees 10 min, 30 cycles of 94 degrees 30 sec, 60 degrees 30sec, and 72 degrees 30 sec, followed by 72 degrees 7 min. PCR products may be purified using QLAquick PCR purification kit (Qiagen), and may be sequenced by the dye-terminator method using PRISM 3700 automated DNA sequencer (Applied Biosystems, Foster City, Calif.) following the manufacturer's instruction outlined in the Owner's Manual (which is hereby incorporated herein by reference in its entirety). Sequencing results may be analyzed for the presence of polymorphisms using PolyPhred software(Nickerson DA 1997; Rieder MJ 1999). All the sequence traces of potential polymorphisms may be visually inspected to confirm the presence of SNPs.

Alternative methods for identifying SNPs of the present invention are known in the art. One such method involves resequencing of target sequences from individuals of diverse ethnic and geographic backgrounds by hybridization to probes immobilized to microfabricated arrays. The strategy and principles for the design and use of such arrays are generally described in WO 95/11995.

A typical probe array used in such an analysis would have two groups of four sets of probes that respectively tile both strands of a reference sequence. A first probe set comprises a plurality of probes exhibiting perfect complementarily with one of the reference sequences. Each probe in the first probe set has an interrogation position that corresponds to a nucleotide in the reference sequence. That is, the interrogation position is aligned with the corresponding nucleotide in the reference sequence, when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets would be identical to the corresponding probe from the first probe set except at the interrogation position, which occurs in the same position in each of the four corresponding probes from the four probe sets, and is occupied by a different nucleotide in the four probe sets. In the present analysis, probes may be nucleotides long. Arrays tiled for multiple different references sequences may be included on the same substrate.

Publicly available sequences for a given gene can be assembled into Gap4 (http://www biozentrum.unibas.ch/-biocomp/staden/Overview.html). PCR primers covering each exon, could be designed, for example, using Primer 3 (httP://www-genome.wi.mit.edu/cgi-bin/primer/primer3.cgi). Primers would not be designed in regions where there are sequence discrepancies between reads. Genomic DNA could be amplified from at least two individuals using 2.5 pmol each primer, 1.5 mM MgCl₂, 100 ˜M dNTPs, 0.75 ˜M AmpliTaq GOLD polymerase, and about 19 ng DNA in a 15 ul reaction. Reactions could be assembled using a PACKARD MultiPROBE robotic pipetting station and then put in MJ 96-well tetrad thermocyclers (96° C. for minutes, followed by cycles of 96° C. for seconds, 59° C. for 2 minutes, and 72° C. for 2 minutes). A subset of the PCR assays for each individual could then be run on 3% NuSieve gels in 0.5× TBE to confirm that the reaction worked.

For a given DNA, 5 ul (about 50 ng) of each PCR or RT -PCR product could be pooled (Final volume=150-200 ul). The products can be purified using QiaQuick PCR purification from Qiagen. The samples would then be eluted once in 35 ul sterile water and 4 ul 10×One-Phor-All buffer (Pharmacia). The pooled samples are then digested with 0.2u DNaseI (Promega) for 10 minutes at 37° C. and then labeled with 0.5 nmols biotin-N-6-ddATP and 15u Terminal Transferase (GibcoBRL Life Technology) for 60 minutes at 37° C. Both fragmentation and labeling reactions could be terminated by incubating the pooled sample for 15 minutes at 100° C.

Low-density DNA chips (Affymetrix, Calif.) may be hybridized following the manufacturer's instructions. Briefly, the hybridization cocktail consisted of 3M TMACI, mM Tris pH 7.8, 0.01% Triton X-100, 100 mg/ml herring sperm DNA {Gibco BRL), 200 pM control biotin-labeled oligo. The processed PCR products are then denatured for 7 minutes at 100° C. and then added to prewarmed {37° C.) hybridization solution. The chips are hybridized overnight at 44° C. Chips are washed in 1× SSPET and 6× SSPET followed by staining with 2 ug/mil SARPE and 0.5 mg/ml acetylated BSA in 200 ul of 6× SSPET for 8 minutes at room temperature. Chips are scanned using a Molecular Dynamics scanner.

Chip image files may be analyzed using Ulysses {Affymetrix, Calif.) which uses four algorithms to identify potential polymorphisms. Candidate polymorphisms may be visually inspected and assigned a confidence value: where high confidence candidates display all three genotypes, while likely candidates show only two genotypes {homozygous for reference sequence and heterozygous for reference and variant). Some of the candidate polymorphisms may be confirmed by ABI sequencing. Identified polymorphisms could then be compared to several databases to determine if they are novel.

### Example 13

### Method of Determining the Allele Frequency for each SNP of the Present Invention

Allele frequencies of these polymorphisms may be determined by genotyping various DNA samples (Coriell Institute; Collingswood, N.J.) using FP-TDI assay (Chen X 1999). Automated genotyping calls may be made with an allele calling software developed by Joel Hirschorn (Whitehead Institute/MIT Center for Genome Research, personal communication).

Briefly, the no template controls (NTCs) may be labeled accordingly in column C. The appropriate cells may be completed in column L indicating whether REF (homozygous ROX) or VAR (homozygous TAMRA) are expected to be rare genotypes (<10% of all samples)—the latter is important in helping the program to identify rare homozygotes. The number of 96 well plates genotyped in cell P2 are noted (generally between 0.5 and 4)—the program works best if this is accurate. No more than 384 samples can be analyzed at a time. The pairs of mP values from the LJL may be pasted into columns E and F; making sure there may be no residual data is left at the bottom fewer than 384 data points are provided. The DNA names may be provided in columns A, B or C; column I will be a concatenation of columns A, B and C. In addition, the well numbers for each sample may be also provided in column D.

With the above information provided, the program should automatically cluster the points and identify genotypes. The program works by converting the mP values into polar coordinates (distance from origin and angle from origin) with the angle being on a scale from 0 to 2; heterozygotes are placed as close to 1 as possible.

The cutoff values in columns L and M may be adjusted as desired.

Expert parameters: The most important parameters are the maximum angle for REF and minimum angle for VAR. These parameters may need to be changed in a particularly skewed assay which may be observed when an REF or VAR cluster is close to an angle of 1 and has called as a failed or HETs.

Other parameters are low and high cutoffs that are used to determine which points are considered for the determination of edges of the clusters. With small numbers of data points, the high cutoff may need to be increased (to 500 or so). This may be the right thing to do for every assay, but certainly when the program fails to identify a small cluster with high signal.

NTC TAMRA and ROX indicate the position of the no template control or failed samples as estimated by the computer algorithm.

No signal=mP< is the threshold below which points are automatically considered failures. “Throw out points with signal above” is the TAMRA or ROX mP value above which points are considered failures. The latter may occasionally need to be adjusted from 250 to 300, but caveat emptor for assays with signals >250. ‘Lump’ or ‘split’ describes a subtle difference in the way points are grouped into clusters. Lump generally is better. ‘HETs expected’ in the rare case where only homozygotes of either class are expected (e.g. a study of X chromosome SNPs in males), change this to “N”.

Notes on method of clustering: The origin is defined by the NTCs or other low signal points (the position of the origin is shown as “NTC TAMRA” and “NTC ROX”); the points with very low or high signal are not considered initially. The program finds the point farthest from the origin and calls that a HET; the ROX/TAMRA ratio is calculated from this point, placing the heterozygotes at 45 degrees from the origin (an angle of “1”). The angles from the origin are calculated (the scale ranges from 0 to 2) and used to define clusters. A histogram of angles is generated. The cluster boundaries are defined by an algorithm that takes into account the shape of the histogram. The homozygote clusters are defined as the leftmost and rightmost big clusters (unless the allele is specified as being rare, in which case the cluster need not be big). The heterozygote is the biggest cluster in between the REF and VAR. If there are two equal clusters, the one best-separated from REF and VAR is called HET. All other clusters are failed. Some fine tuning is applied to lump in scattered points on the edges of the clusters (if “Lump” is selected). The boundaries of the clusters are “Angles” in column L.

Once the clusters are defined, the interquartile distance of signal intensity is defined for each cluster. Points falling more than 3 or 4 interquartiles from the mean are excluded. (These are the “Signal cutoffs” in column M).

Allele frequency of the B1 receptor R317Q variant (AE103s1) is as follows. 7% in African Americans (7/94), 0% in Caucasians (0/94), 0% in Asians (0/60), and 0% in Amerindians (0/20). Higher frequency of this form in African Americans than in Caucasians matches the profile of a potential genetic risk factor for angioedema, which is observed more frequently in African Americans than in Caucasians (Brown N.J. 1996; Brown N.J. 1998; Agostoni A 1999; Coats 2000).

The invention encompasses additional methods of determining the allelic frequency of the SNPs of the present invention. Such methods may be known in the art, some of which are described elsewhere herein.

### Example 14

### Alternative Methods of Detecting Polymorphisms Encompassed by the Present Invention

### Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H.A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86, 1173 (1989), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Additional methods of amplification are known in the art or are described elsewhere herein.

### Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points ofvariation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. Additional methods of analysis are known in the art or are described elsewhere herein.

### Allele-Specific Probes

The design and use of allele-specific probes for analyzing polymorphisms is described by e.g., Saiki et al., Nature 324,163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### Tiling Arrays

The polymorphisms can also be identified by hybridization to nucleic acid arrays, some examples of which are described in WO 95/11995. The same arrays or different arrays can be used for analysis of characterized polymorphisms. WO 95/11995 also describes sub arrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to bases).

### Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17,2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., WO 93/22456).

### Direct-Sequencing

The direct analysis of the sequence of polymorphisms of the present invention can be accomplished using either the dideoxy chain termination method or the Maxam—Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology. Principles and Applications for DNA Amplification, (W .H. Freeman and Co, New York, 1992), Chapter 7.

### Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86,2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

### Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), uses a locus-specific oligonucleotide primer labeled on the 5′ terminus with 5-carboxyfluorescein (F AM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 15

### Method of Assessing the Expression Profile of the Novel HGPRBMY8 Polypeptides of the Present Invention using Expanded mRNA Tissue and Cell Sources

Total RNA from tissues was isolated using the TriZol protocol (Invitrogen) and quantified by determining its absorbance at 260 nM. An assessment of the 18s and 28s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For HGPRBMY8, the primer probe sequences were as follows:

| | |
|---|---|
| Forward Primer | |
| 5′-TTCCTCGCCGCCTCTTTC-3′ | (SEQ ID NO:105) |
| Reverse Primer | |
| 5′-CGGCTTGCGCTGCAA-3′ | (SEQ ID NO:106) |
| TaqMan Probe | |
| 5′-ACTAGCGCCAGCACTATGTTGCCGA-3′ | (SEQ ID NO:107) |

For HGPRBMY34, the primer probe sequences were as follows

| | |
|---|---|
| Forward Primer | |
| 5′-CACAGCCATCAACTTCTTCCTCTA-3′ | (SEQ ID NO:108) |
| Reverse Primer | |
| 5′-GGCGGCTGCCATGGT-3′ | (SEQ ID NO:109) |
| TaqMan Probe | |
| 5′-TGCTTCATCAGCAAGCGGTTCCG-3′ | (SEQ ID NO:110) |

For glutamate decarboxylase, the primer probe sequences were as follows

| | |
|---|---|
| Forward Primer | |
| 5′-AAGTGGATGTCAACTACGCGTTT-3′ | (SEQ ID NO:111) |
| Reverse Primer | |
| 5′-CGCCAAAGTGGGCCTTT-3′ | (SEQ ID NO:112) |
| TaqMan Probe | |
| 5′-CCATGCAACAGACCTGCTGCCG-3′ | (SEQ ID NO:113) |

### DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+RNA. If not the RNA was not used in actual experiments.

### Reverse Transcription reaction and Sequence Detection

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq Gold™. The PCR reaction was then held at 94° C. for 12 min, followed by 40 cycles of 94° C. for 15 sec and 60° C. for 30 sec.

### Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}

The expanded expression profile of the HGPRBMY8 polypeptide is provided in FIG. 19 and described elsewhere herein.

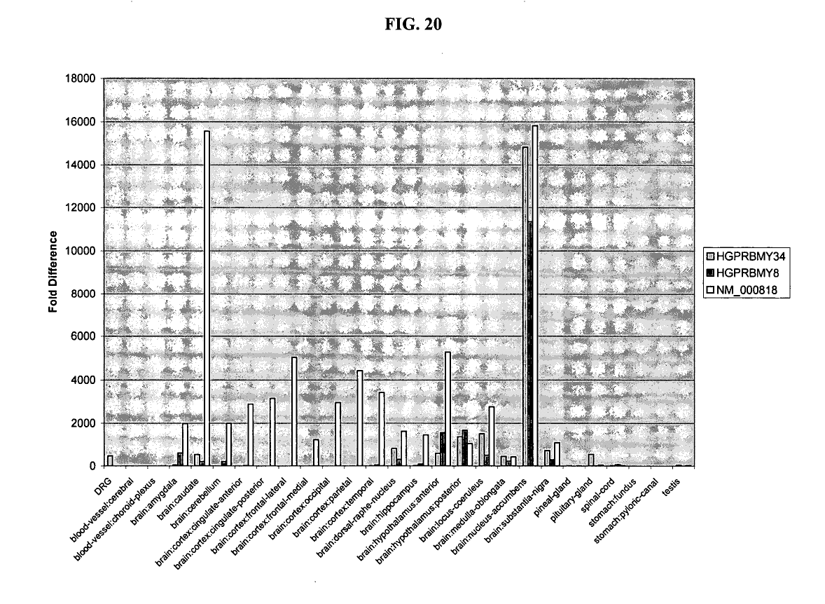

The combined expression profile of the HGPRBMY8 polypeptide with HGPRBMY34, and glutamate decarboxylase, is provided in FIG. 20 and described elsewhere herein.

### Example 16

### Protein Coupled Receptor Immunohistochemistry Hybridization Expression Profiling

Immunohistochemistry expression using the LifeSpan database, describes positive staining in tumor cells from ovarian carcinoma, colonic adenocarcinoma, pancreatic carcinoma, lung adenocarcinoma, breast carcinoma, and melanoma. Slides containing paraffin sections (LifeSpan BioSciences, Inc.; Seattle, Wash.) were deparaffinized through xylene and alcohol, rehydrated, and then subjected to the steam method of target retrieval (#S1700; DAKO Corp.; Carpenteria, Calif.).

Immunohistochemical assay techniques are commonly known in the art and are described briefly herein. Immunocytochemical (ICC) experiments were performed on a DAKO autostainer following the procedures and reagents developed by DAKO. Specifically, the slides were blocked with avidin, rinsed, blocked with biotin, rinsed, protein blocked with DAKO universal protein block, machine blown dry, primary antibody, incubated, and the slides rinsed. Biotinylated secondary antibody was applied using the manufacturer's instructions (1 drop/10 ml, or approximately 0.75 μg/nL), incubated, rinsed slides, and applied Vectastain ABC-AP reagent for 30 minutes. Vector Red was used as substrate and prepared according to the manufacturer's instructions just prior to use.

The sequence for HGPRBMY8 was analyzed by the algorithm of Hopp and Woods to determine potential peptides for synthesis and antibody production. The peptides were then blasted against the Swissprot database to determine uniqueness, and to help predict the specificity of the resulting antibodies. Peptide MHKTIKKEIQDMLKKFFC (SEQ ID NO:114) was selected and synthesized, and rabbit polyclonal antisera were generated. The peptide was conjugated to the carrier protein (KLH) by the C-terminal cysteine residue. The third bleeds were subjected to peptide affinity purification, and the resulting antisera were then used as primary antibodies in immunohistochemistry experiments.

Antibody titration experiments were conducted with antibody HGPRBMY8 (rabbit polyclonal) to establish concentrations that would result in minimal background and maximal detection of signal. Serial dilutions were performed at 1:50, 1:100, 1:250, 1:500, and 1:1000. The serial dilution study demonstrated the highest signal-to-noise ratios at dilutions of 1:100 and 1:250 on paraffin-embedded, formalin- fixed tissues. These concentrations were used for the study. Antibody HGPRBMY8 was used as the primary antibody, and the principal detection system consisted of a Vector anti-rabbit secondary (BA-1000), a Vector ABC-AP Kit (AK-5000) with a Vector Red substrate kit (SK-5100), which was used to produce a fuchsia-colored deposit. Tissues were also stained with a positive control antibody (CD31) to ensure that the tissue antigens were preserved and accessible for immunohistochemical analysis. Only tissues that stained positive for CD31 were chosen for the remainder of this study. The negative control consisted of performing the entire immunohistochemistry procedure on adjacent sections in the absence of primary antibody. Slides were imaged using a DVC 1310C digital camera coupled to a Nikon microscope.

The results of this study are consistent with the expression results outlined elsewhere herein. Briefly, this study showed that antibody directed to HGPRBMY8 selectively stained the following tissues extensively: the neuropil of the amygdala, the amygdalotemporal cortex, the inferior-temporal cortex, the orbitofrontal cortex, the entorhinal cortex, the subiculum, areas CA1 through CA4, the hypothalamus, the substantia nigra, the hypoglossal, solitary, gracile, cuneate, lateral cuneate, trigeminal, arcuate, and olivary nuclei in the medulla, and the nucleus of Clarke in the spinal cord. Many neurons in the amygdala, the caudate, the putamen, the basal striatum, the claustrum, hippocampal areas CA1 through CA4, the cortex, the substantia innominata, the nucleus basalis of Meynert, the paraventricular, tuberal, posterior hypothalamic and posterior lateral hypothalamic nuclei, and the thalamus were stained strongly. Many neurons in the orbitofrontal cortex and the amygdalotemporal cortex showed faint to moderate staining. Stellate, basket, and Purkinje neurons in the cerebellum, the lateral geniculate body and nuclei in the medulla showed faint to moderate staining. Rarely, strong staining was found in protoplasmic astrocytes—a subset of astrocytes intimately associated with neurons—in the amygdala, hippocampus, cerebral cortex, and anterior nuclear group of the thalamus. A few myelinated nerve tracts or fibers showed faint staining in a sample of thoracic spinal cord. Oligodendrocytes and microglia were negative. The choroid plexus epithelium showed moderate to strong staining. Ependymal cells showed faint to moderate staining. In the pituitary, the pars intermedia and Herring bodies in the pars posterior stained strongly. Generally, vascular smooth muscle cells stained moderately. In conclusion, this antibody mainly stained neurons and neuropil in many brain samples.

### Example 17

### Complementary Polynucleotides

Antisense molecules or nucleic acid sequences complementary to the HGPRBMY8 protein-encoding sequence, or any part thereof, is used to decrease or to inhibit the expression of naturally occurring HGPRBMY8. Although the use of antisense or complementary oligonucleotides comprising about 15 to 35 base-pairs is described, essentially the same procedure is used with smaller or larger nucleic acid sequence fragments. An oligonucleotide based on the coding sequence of HGPRBMY8 protein, as shown in FIGS. 1 and 2, or as depicted in SEQ ID NO:1, for example, is used to inhibit expression of naturally occurring HGPRBMY8. The complementary oligonucleotide is typically designed from the most unique 5′ sequence and is used either to inhibit transcription by preventing promoter binding to the coding sequence, or to inhibit translation by preventing the ribosome from binding to the HGPRBMY8 protein-encoding transcript, among others. However, other regions may also be targeted.

Using an appropriate portion of the signal and 5′ sequence of SEQ ID NO:1, an effective antisense oligonucleotide includes any of about 15-35 nucleotides spanning the region which translates into the signal or 5′ coding sequence, among other regions, of the polypeptide as shown in FIG. 1 (SEQ ID NO:1). Appropriate oligonucleotides are designed using OLIGO 4.06 software and the HGPRBMY8 protein coding sequence (SEQ ID NO:1). Preferred oligonucleotides are deoxynucleotide, or chimeric deoxynucleotide/ribonucleotide based and are provided below. The oligonucleotides were synthesized using chemistry essentially as described in U.S. Pat. No. 5,849,902; which is hereby incorporated herein by reference in its entirety.

| | |
|---|---|
| ID # | Sequence |

| | | |
|---|---|---|
| 12681 | GUGAGACAGGUUGCAGGCUCAGUGC | (SEQ ID NO:115) |
| 12682 | AACCAGCACGGUUGAGCGGAUGAUG | (SEQ ID NO:116) |
| 12683 | GAUCCACUGACACCACGACAAUGGU | (SEQ ID NO:117) |
| 12684 | UGAAGGACACCACGCUGAGAAUAGU | (SEQ ID NO:118) |
| 12685 | GGAGUAGCAGGCAAUCAUGACAAUC | (SEQ ID NO:119) |
| 13028 | GCTGACTCGGACGTTGGACAGAGTG | (SEQ ID NO:120) |
| 13029 | GTGTAAGGGCAGTTGGCACGACCAA | (SEQ ID NO:121) |
| 13030 | TTGGAACAGCACCACAGTCACCTAG | (SEQ ID NO:122) |
| 13031 | TGATAAGAGTCGCACCACAGGAAGT | (SEQ ID NO:123) |
| 13032 | CTAACAGTACTAACGGACGATGAGG | (SEQ ID NO:124) |

The HGPRBMY8 polypeptide has been shown to be involved in the regulation of mammalian cell cycle pathways. Subjecting cells with an effective amount of a pool of all five of the above antisense oligoncleotides resulted in a significant increase in p21 expression/activity providing convincing evidence that HGPRBMY8 at least regulates the activity and/or expression of p21 either directly, or indirectly. Moreover, the results suggest that HGPRBMY8 is involved in the negative regulation of p21 activity and/or expression, either directly or indirectly. The p21 assay used is described below and was based upon the analysis of p21 activity as a downstream marker for proliferative signal transduction events.

Moreover, subjecting cells with an effective amount of a pool of all five of the above antisense oligoncleotides resulted in a significant decrease in p27 expression/activity providing convincing evidence that HGPRBMY8 at least regulates the activity and/or expression of p27 either directly, or indirectly. Moreover, the results suggest that HGPRBMY8 is involved in the positive regulation of p27 activity and/or expression, either directly or indirectly. The p27 assay used is described below and was based upon the analysis of p27 activity as a downstream marker for proliferative signal transduction events.

In addition, the HGPRBMY8 polypeptide has been shown to be involved in the regulation of mammalian NF-κB and apoptosis pathways. Subjecting cells with an effective amount of a pool of all five of the above antisense oligoncleotides resulted in a significant decrease in IκBα expression/activity providing convincing evidence that HGPRBMY8 at least regulates the activity and/or expression of IκBα either directly, or indirectly. Moreover, the results suggest that HGPRBMY8 is involved in the positive regulation of NF-κB/IκBα activity and/or expression, either directly or indirectly. The IκBα assay used is described below and was based upon the analysis of IκBα activity as a downstream marker for proliferative signal transduction events.

### Transfection of Post-Quiescent A549 Cells with AntiSense Oligonucleotides

Materials Needed:

A549 cells maintained in DMEM with high glucose (Gibco-BRL) supplemented with 10% Fetal Bovine Serum, 2 mM L-Glutamine, and 1×penicillin/streptomycin.

Opti-MEM (Gibco-BRL)

Lipofectamine 2000 (Invitrogen)

Antisense oligomers (Sequitur)

Polystyrene tubes.

Tissue culture treated plates.

Quiescent Cells were Prepared as Follows:

Day 0: 300,000 A549 cells were seeded in a T75 tissue culture flask in 10 ml of A549 media (as specified above), and incubated in at 37° C., 5% CO₂ in a humidified incubator for 48 hours.

Day 2: The T75 flasks were rocked to remove any loosely adherent cells, and the A549 growth media removed and replenished with 10 ml of fresh A549 media. The cells were cultured for six days without changing the media to create a quiescent cell population.

Day 8: Quiescent cells were plated in multi-well format and transfected with antisense oligonucleotides.

A549 Cells were Transfected According to the Following:

1. Trypsinize T75 flask containing quiescent population of A549 cells.

2. Count the cells and seed 24-well plates with 60K quiescent A549 cells per well.

3. Allow the cells to adhere to the tissue culture plate (approximately 4 hours).

4. Transfect the cells with antisense and control oligonucleotides according to the following:

a. A 10× stock of lipofectamine 2000 (10 ug/ml is 10×) was prepared, and diluted lipid was allowed to stand at RT for 15 minutes.

Stock solution of lipofectamine 2000 was 1 mg/ml.

10× solution for transfection was 10 ug/ml.

To prepare 10× solution, dilute 10 ul of lipofectamine 2000 stock per 1 ml of Opti-MEM (serum free media).

b. A 10× stock of each oligomer was prepared to be used in the transfection.

Stock solutions of oligomers were at 100 uM in 20 mM HEPES, pH 7.5.

10× concentration of oligomer was 0.25 uM.

To prepare the 10× solutions, dilute 2.5 ul of oligomer per 1 ml of Opti-MEM.

c. Equal volumes of the 10× lipofectamine 2000 stock and the 10× oligomer solutions were mixed well, and incubated for 15 minutes at RT to allow complexation of the oligomer and lipid. The resulting mixture was 5×.

d. After the 15 minute complexation, 4 volumes of full growth media was added to the oligomer/lipid complexes (solution was 1×).

e. The media was aspirated from the cells, and 0.5 ml of the 1× oligomer/lipid complexes added to each well.

f. The cells were incubated for 16-24 hours at 37° C. in a humidified CO₂ incubator.

g. Cell pellets were harvested for RNA isolation and TaqMan analysis of downstream marker genes.

### TaqMan Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

### Dnase I Treatment

| Reagent | Per r'xn (in uL) |
|---|---|
| 10 × Buffer | 2.5 |
| Dnase I (1 unit/ul @ 1 unit per ug sample) | 2 |
| DEPC H₂O | 0.5 |
| RNA sample @ 0.1 ug/ul | 20 |
| (2-3 ug total) | |
| Total | 25 |

Next, 5 ul of master mix was aliquoted per well of a 96-well PCR reaction plate (PE part # N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part # N801-0935), placed in a plate, and briefly spun in a plate centrifuge (Beckman) to collect all volume in the bottom of the tubes. Generally, a short spin up to 500 rpm in a Sorvall RT is sufficient The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

### RT reaction

A master mix of reagents was prepared according to the following table:

### RT reaction

| | RT | | No RT | |
|---|---|---|---|---|
| Reagent | Per Rx'n (in ul) | | Per Rx'n (in ul) | |
| 10 × RT buffer | 5 | | 2.5 | |
| MgCl₂ | 11 | | 5.5 | |
| DNTP mixture | 10 | | 5 | |
| Random Hexamers | 2.5 | | 1.25 | |
| Rnase inhibitors | 1.25 | | 0.625 | |
| RT enzyme | 1.25 | | — | |
| Total RNA 500 ng | 19.0 | max | 10.125 | max |
| (100 ng no RT) | | | | |
| DEPC H₂O | — | | — | |
| Total | 50 | uL | 25 | uL |

Samples were adjusted to a concentration so that 500 ng of RNA was added to each RT rx'n (10 ng for the no RT). A maximum of 19 ul can be added to the RT rx'n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part # N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for generation of a standard curve.

The wells were capped using strip well caps (PE part # N801-0935), placed in a plate, and spin briefly in a plate centrifuge (Beckman) to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @−20° C. or lower upon completion.

### p21 TaqMan Reaction (Template Comes from RT Plate.)

A master mix was prepared according to the following table:

### TaqMan Reaction (per well)

| Reagent | Per Rx'n (in ul) |
|---|---|
| TaqMan Master Mix | 4.17 |
| 100 uM Probe (SEQ ID NO: 127) | .025 |
| 100 uM Forward primer (SEQ ID NO: 125) | .05 |
| 100 uM Reverse primer (SEQ ID NO: 126) | .05 |
| Template | — |
| DEPC H₂O | 18.21 |
| Total | 22.5 |

The primers used for the RT-PCR reaction is as follows:

P21 primer and probes

| | |
|---|---|
| Forward Primer: | |
| CTGGAGACTCTCAGGGTCGAA | (SEQ ID NO:125) |
| Reverse Primer: | |
| GCGCTTCCAGGACTGCA | (SEQ ID NO:126) |
| TaqMan Probe: | |
| ACAGATTTCTACCACTCCAAACGCCGG | (SEQ ID NO:127) |

### p27 TaqMan Reaction (Template Comes from RT Plate.)

A master mix was prepared according to the following table:

### TaqMan Reaction (per well)

| Reagent | Per Rx'n (in ul) |
|---|---|
| TaqMan Master Mix | 4.17 |
| 100 uM Probe (SEQ ID NO: 130) | .025 |
| 100 uM Forward primer (SEQ ID NO: 128) | .05 |
| 100 uM Reverse primer (SEQ ID NO: 129) | .05 |
| Template | — |
| DEPC H₂O | 18.21 |
| Total | 22.5 |

The primers used for the RT-PCR reaction is as follows:

### P21 Primer and Probes

| | |
|---|---|
| Forward Primer: | |
| CTGGAGACTCTCAGGGTCGAA | (SEQ ID NO:128) |
| Reverse Primer: | |
| GCGCTTCCAGGACTGCA | (SEQ ID NO:129) |
| TaqMan Probe: | |
| ACAGATTTCTACCACTCCAAACGCCGG | (SEQ ID NO:130) |

### IkB TaqMan Reaction (Template Comes from RT Plate.)

A master mix was prepared according to the following table:

### TaqMan Reaction (per well)

| Reagent | Per Rx'n (in ul) |
|---|---|
| TaqMan Master Mix | 4.17 |
| 100 uM Probe (SEQ ID NO: 133) | .025 |
| 100 uM Forward primer (SEQ ID NO: 131) | .05 |
| 100 uM Reverse primer (SEQ ID NO: 132) | .05 |
| Template | — |
| DEPC H₂O | 18.21 |
| Total | 22.5 |

The Primers Used for the RT-PCR Reaction is as Follows:

### P21 Primer and Probes

| | |
|---|---|
| Forward Primer: | |
| CTGGAGACTCTCAGGGTCGAA | (SEQ ID NO:131) |
| Reverse Primer: | |
| GCGCTTCCAGGACTGCA | (SEQ ID NO:132) |
| TaqMan Probe: | |
| ACAGATTTCTACCACTCCAAACGCCGG | (SEQ ID NO:133) |

Using a Gilson P-10 repeat pipetter, 22.5 ul of master mix was aliquouted per well of a 96-well optical plate. Then, using P-10 pipetter, 2.5 ul of sample was added to individual wells. Generally, RT samples are run in triplicate with each primer/probe set used, and no RT samples are run once and only with one primer/probe set, often gapdh (or other internal control).

A standard curve is then constructed and loaded onto the plate. The curve has five points plus one no template control (NTC, =DEPC treated H₂O). The curve was made with a high point of 50 ng of sample (twice the amount of RNA in unknowns), and successive samples of 25, 10, 5, and 1 ng. The curve was made from a control sample(s) (see above).

The wells were capped using optical strip well caps (PE part # N801-0935), placed in a plate, and spun in a centrifuge to collect all volume in the bottom of the tubes. Generally, a short spin up to 500 rpm in a Sorvall RT is sufficient.

Plates were loaded onto a PE 5700 sequence detector making sure the plate is aligned properly with the notch in the upper right hand corner. The lid was tightened down and run using the 5700 and 5700 quantitation program and the SYBR probe using the following thermal profile:

50° C. for 2 min

95° C. for 10 min

and the following for 40 cycles:

95° C. for 15 sec

60° C. for 1 min

Change the reaction volume to 25 ul.

Once the reaction was complete, a manual threshold of around 0.1 was set to minimize the background signal. Additional information relative to operation of the GeneAmp 5700 machine may be found in reference to the following manuals: “GeneAmp 5700 Sequence Detection System Operator Training CD”; and the “User's Manual for 5700 Sequence Detection System”; available from Perkin-Elmer and hereby incorporated by reference herein in their entirety.

### Example 18

### Screening Methods to Identify Modulators of the HGPRBMY8 Polypeptide

### Introduction

G protein-coupled receptors (GPCRs) are a superfamily of seven transmembrane-spanning proteins that are activated by a wide range of extracellular ligands, including small molecules such as biogenic amines, amino acids, ions, small and large peptides, and bioactive lipids. GPCRs are expressed in virtually all tissues and are involved in the regulation of a variety of cellular and physiological responses, such as neurotransmission, chemotaxis, inflammation, cell proliferation, cardiac and smooth muscle contractility, and visual and chemosensory perception (Bockaert et al., 2002; Pierce et al., 2002).

The sequencing of the human genome has led to predictions that as many as 1,000 of the 35,000-60,000 human genes encode G-protein coupled receptors (Lander et al., 2001; Venter et al., 2001). About 400 of these are non-chemosensory receptors and can therefore be considered as potential drug targets. More than half of the so-called “druggable” GPCRs are known receptors, in the sense that their activating ligands have been identified. The remaining ˜155 receptors are “orphan receptors” (oGPCRs), for which the natural activating ligands remain unknown. While there has been recent success in identifying endogenous ligands for some oGPCRs, most remain without a cognate ligand despite substantial effort.

The most common approach for de-orphanizing thus far is to use “reverse pharmacology” to screen populations of mammalian cells transiently transfected with the oGPCR of interest. The reverse pharmacological strategy has resulted in the discovery of more than 50 ligands for orphan GPCRs (Szekeres, 2002). Candidate compounds for screening can be selected based on similarity of the orphan to receptors of known pharmacology. Alternatively, activating ligands can be identified using mixtures of fractionated tissue extracts, often prepared from tissues known to express the oGPCR in question (Civelli, 1998; Hinuma et al., 1999). Finally, diverse collections of known and presumptive GPCR signaling molecules can be assembled and screened in bulk for modulators of orphan receptor function. Also, since the signal transduction pathway(s) to which a given oGPCR naturally couples is unknown, orphan GPCRs are frequently screened in the presence of co-expressed promiscuous G protein alpha subunits, Gα₁₅/Gα₁₆, or various chimeric Gα subunits in order to re-direct receptor signal output to predefined endpoints (Milligan et al., 1996; Milligan and Rees, 1999; Offermanns and Simon, 1995).

The inherent difficulty in identifying stable cell lines exhibiting expression and coupling of oGPCRs to signal transduction pathways in mammalian cell lines has forced most oGPCR screening to rely on transient transfection systems. Transient expression of an oGPCR has certain advantages, namely, that following transfection, oGPCRs are generally expressed at reasonably high levels at the cell surface. However, the logistical challenges involved in scaling transient transfection protocols to support high throughput screening (HTS) has necessitated smaller, more focused screening strategies for orphan GPCR ligand discovery (Howard et al., 2001).

One of the many challenges in working with orphan GPCRs within the pharmaceutical industry is managing the expense of screening targets which have no clear function or role in disease. By utilizing technology and parallel processing of multiple orphan GPCRs in a single screening campaign, it is possible to minimize the risk by decreasing costs associated with reagent consumption and staffing. This approach also reduces cost by reducing the number of follow-up assays and selectivity tests that need to be performed.

Described herein is the successful application of focused screening methods to the identification of selective activators of the human Family A orphan GPCR, HGPRBMY8.

### Materials and Methods

Compounds and Compound Screening Plates. The orphan receptor deorphanizing ligand library was constructed from commercially available GPCR compound libraries. The Neurotransmitter, Bioactive Lipid, and Orphan Ligand Libraries were obtained from BIOMOL Research Laboratories (Plymouth Meeting, Pa.), and the GPCR Peptide Ligand Library was purchased from Phoenix Pharmaceuticals (Belmont, Calif.). All other compounds were purchased from Sigma-Aldrich (St. Louis, Mo.). Prior to use, the peptides in the GPCR Peptide Ligand Library were diluted to 10 μM final concentration using the manufacturer's peptide storage buffer. The concentration and storage buffer of the molecules in the commercial libraries varied but was typically 10 mM in DMSO. The commercially obtained 96 well compound plates were reformatted into 384 well storage plates. For screening, GPCR compound libraries containing in-house synthesized molecules, along with several hundred known GPCR agonists present in the Bristol-Myers Squibb compound screening deck, were added to the deorphanizing ligand library (in-house compound concentration 1 mM in 100% DMSO). All compound plates destined for primary screening were replica plated and stored at −80° C. until use.

Cloning and Expression of Orphan Receptor HGPRBMY8. Orphan GPCR cDNAs were cloned in the expression vector pcDNA3.1 (Invitrogen Life Technologies, Carlsbad Calif.), as described in Example 7 herein.

Cell Culture. All cell culture media and supplements were purchased from Invitrogen Life Technologies (Grand Island, N.Y.). HEK293 cells were maintained in modified Eagle's medium supplemented with 10% FBS and were routinely propagated by subculturing on a biweekly schedule.

Transient Transfection. HEK293 cells were seeded at 8×10⁶ cells per T-175 cm² tissue culture flask one day prior to transient transfections. Transfections were performed with 12 μg of total DNA per flask using the Lipofectamine PlUSTM reagent (Invitrogen Life Technologies, Grand Island, N.Y.) according to the manufacturer's instructions. Cells were transfected with a 50:50 mixture of pcDNA3-Gα15 and pcDNA3.1-oGPCR plasmids, or pcDNA3.1-Topaz (evaluation of transfection efficiency). Twenty-four hours after transfection, cells co-transfected with orphan GPCR cDNAs+Gα15 were harvested, counted and seeded into 384 well plates for the calcium imaging assay (see below). Cells transfected with Topaz were harvested and transfection efficiency was determined by FACS using the FACS Vantage SE (Becton Dickinson, Franklin Lakes, N.J.). All plasmids were prepared using standard methods (Sambrook and Russell, 2001). Compound selectivity toward HGPRBMY8 was provided by testing the same compound sets on HEK293 cells transfected with the orphan GPCR cDNA, RAI-3 (SEQ ID NO:134; Co-penidng U.S. Ser. No. 10/600,816; Filed June 20^{th}, 2003; which is hereby incorporated by reference in its entirety herein). Cells transfected with the selectivity receptor were well-matched to the HGPRBMY8 transfected cells in terms of their response to a control stimulus, (100 μM adenosine triphosphate).

Calcium Imaging Assay. Transiently transfected HEK293 cells were detached with Cell Dissociation Buffer, resuspended in phenol red-free Dulbecco's modified Eagle's medium supplemented with 20 mM HEPES, pH 7.2, and plated at 15,000 cells per well in poly-d-lysine coated 384 well assay plates (Coming Life Sciences, Acton, Mass.). Following an overnight incubation at 37° C./5% CO₂, cells were loaded for 90 minutes with the cell-permeable calcium indicator, Fluo-4 (TEF Labs, Austin, Tex.). The loading solution (3×) contained 10 μg/ml Fluo-4 and 0.5 mg/ml pluronic F127 in Hanks Balanced Salt Solution supplemented with 20 mM HEPES. The assay was initiated by the addition of 20 μL of a 2.5× compound solution, diluted in the HEPES-buffered Hanks solution. Control wells received dilution buffer only (negative controls) or a challenge dose of adenosine triphosphate (ATP, 100 μM final concentration), an activator of endogenous HEK293 GPCRs that are coupled to calcium mobilization. Changes in intracellular calcium concentration in response to compound addition were imaged using a Molecular Devices FLIPR™. The final compound concentrations in the FLIPR primary screening assay was 160 μM, 16 μM, and 160 nM for the commercial compounds, in-house library compounds, and commercial peptides, respectively.

Statistical Calculations. The following statistical measures were used to calculate assay robustness (x₁ and X₃ are the means of the minimum and maximum signals, and s₁ and s₃ are the respective standard deviations):

$$\begin{matrix}{Z}^{′} = 1 - ( \frac{3 {s}_{3} + 3 {s}_{1}}{{\overline{x}}_{3} - {\overline{x}}_{1}} ) \\ Signal Window = \frac{( {\overline{x}}_{3} - 3 {s}_{3} ) - ( {\overline{x}}_{1} + 3 {s}_{1} )}{\sqrt{\frac{( {s}_{1}^{2} + {s}_{3}^{2} )}{2}}} \\ Signal \text{-} to \text{-} Noise = \frac{( {\overline{x}}_{3} - {\overline{x}}_{1} )}{\sqrt{{s}_{3}^{2} + {s}_{1}^{2}}}\end{matrix}$$

### Results and Discussion

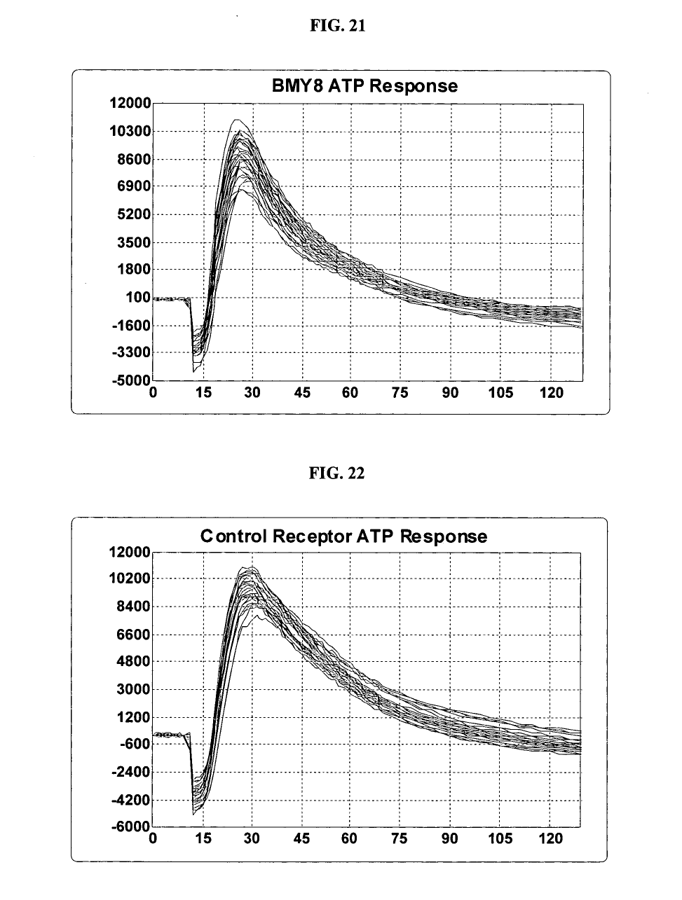

Compound Identification—Calcium Imaging Assay. HEK293 cells transiently co-transfected with expression cDNAs encoding HGPRBMY8 and the promiscuous Gα subunit, Gα15, were screened using a collection of 4,328 deorphanizing ligands in a focused screening campaign. The screen included a selectivity control consisting of HEK293 cells transiently transfected with an unrelated orphan GPCR (Family C family member, RAI-3; SEQ ID NO:134) that was screened in parallel using the same deorphanizing library. The selectivity control was well-matched to the HGPRBMY8 test cells in terms of its response to a control stimulus of 100 μM ATP (see FIGS. 21 and 22).

This parallel approach permitted the identification of any non-selective activators acting through endogenous receptors, or via a non-receptor dependent mechanism. Hits were defined as compounds having activity greater than three standard deviations from the sample mean. Assay quality was determined using the calcium response for cell treated with the positive control stimulus (100 μM ATP) and the negative control stimulus (compound dilution buffer) as the high and low signals, respectively. The Z′ for the calcium imaging assay ranged from 0.41 and 0.66 for HGPRBMY8 transfected cells, and between 0.40 and 0.69 for the control receptor, RAI3 (see Table 2).

TABLE 2

| | | | | | | | | | | |
|---|---|---|---|---|---|---|---|---|---|---|
| Plate1 | Plate2 | Plate3 | Plate4 | Plate5 | Plate6 | Plate7 | Plate8 | Plate9p | Plate10 | Plate11 |

BMY8 Assay Statistics

| | | | | | | | | | | | |
|---|---|---|---|---|---|---|---|---|---|---|---|
| S:N | 6.43 | 6.03 | 7.33 | 7.01 | 5.71 | 10.27 | 6.71 | 6.37 | 5.96 | 6.23 | 8.42 |
| SigWin | 4.23 | 3.76 | 4.97 | 5.00 | 3.34 | 9.55 | 4.72 | 4.34 | 3.75 | 14.15 | 7.19 |
| Z′ | 0.46 | 0.44 | 0.48 | 0.50 | 0.41 | 0.66 | 0.50 | 0.48 | 0.45 | 0.47 | 0.60 |

Control Receptor Assay Statistics

| | | | | | | | | | | | |
|---|---|---|---|---|---|---|---|---|---|---|---|
| S:N | 11.69 | 5.96 | 6.21 | 6.90 | 7.54 | 8.76 | 5.55 | 7.77 | 9.68 | 7.71 | 8.66 |
| SigWin | 11.43 | 3.84 | 3.54 | 4.66 | 5.68 | 7.13 | 3.11 | 6.10 | 8.61 | 5.90 | 7.33 |
| Z′ | 0.69 | 0.46 | 0.40 | 0.48 | 0.53 | 0.58 | 0.40 | 0.56 | 0.63 | 0.54 | 0.60 |

Twelve small molecules from the deorphanizing library appeared to selectively activate the HGPRBMY8+Gα15 transfected cells, compared to its matched selectivity control, RAI-3 (at least two-fold greater activity in HGPRBMY8). Interestingly, these molecules were all from various combinatorial chemical libraries designed to target GPCRs. The results are presented in Table 3.

TABLE 3

| Compound | Activity HGPRBMY8^{a} | Activity Control^{b} |
|---|---|---|
| A | 11.4 | −0.6 |
| B | 12.4 | 1.5 |
| C | 11.5 | −1.5 |
| D | 16.4 | 2.4 |
| E | 11.6 | 1.7 |
| F | 10.1 | 2.0 |
| G | 14.1 | 3.5 |
| H | 10.1 | 2.8 |
| I | 10.4 | 3.6 |
| J | 12.3 | 4.6 |
| K | 11.5 | 5.1 |
| L | 11.0 | 4.9 |

^{a}Percent activity of test compound relative to a challenge stimulus of 100 μM ATP.
^{b}Control HEK293 cells transfected with the orphan GPCR, RAI-3.

The molecules identified in this focused screen represent a starting point for small molecule drug discovery for the human orphan GPCR, HGPRBMY8.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

### REFERENCES

1. Rees, S., Brown, S., Stables, J.: “Reporter gene systems for the study of G Protein Coupled Receptor signalling in mammalian cells”. In Milligan G. (ed.): Signal Transduction: A practical approach. Oxford: Oxford University Press, 1999: 171-221.

2. Alam, J., Cook, J. L.: “Reporter Genes: Application to the study of mammalian gene transcription”. Anal. Biochem. 1990; 188: 245-254.

3. Selbie, L. A. and Hill, S. J.: “G protein-coupled receptor cross-talk: The fine-tuning of multiple receptor-signaling pathways”. TiPs. 1998; 19: 87-93.

4. Boss, V., Talpade, D.J., and Murphy, T.J.: “Induction of NFAT mediated transcription by Gq-coupled Receptors in lympoid and non-lymphoid cells”. JBC. 1996; 271: 10429-10432.

5. George, S. E., Bungay, B. J., and Naylor, L. H.: “Functional coupling of endogenous serotonin (5-HT1B) and calcitonin (C1a) receptors in CHO cells to a cyclic AMP-responsive luciferase reporter gene”. J. Neurochem. 1997; 69: 1278-1285.

6. Suto, C M, Igna DM: “Selection of an optimal reporter for cell-based high throughput screening assays”. J. Biomol. Screening. 1997; 2: 7-12.

7. Zlokarnik, G., Negulescu, P.A., Knapp, T.E., More, L., Burres, N., Feng, L., Whitney, M., Roemer, K., and Tsien, R. Y. “Quantitation of transcription and clonal selection of single living cells with a B-Lactamase Reporter”. Science. 1998; 279: 84-88.

8. S. Fiering et. al., Genes Dev. 4, 1823 (1990).

9. J. Karttunen and N. Shastri, PNAS 88, 3972 (1991).

10. Hawes, B. E., Luttrell. L. M., van Biesen, T., and Lefkowitz, R. J. (1996) JBC 271, 12133-12136.

11. Gilman, A. G. (1987) Annul. Rev. Biochem. 56, 615-649.

12. Maniatis et al., Cold Spring Harbor Press, 1989.

13. Salcedo, R., Ponce, M. L., Young, H. A., Wasserman, K., Ward, J. M., Kleinman, H. K., Oppenheim, J. J., Murphy, W. J. “Human endothelial cells express CCF2 and respond to MCP-1: direct role of MCP-1 in angiogenesis and tumor progression”. Blood. 2000; 96 (1): 34-40.

14. Sica, A., Saccani, A., Bottazzi, B., Bemasconi, S., Allavena, P., Gaetano, B., LaRossa, G., Scotton, C., Balkwill F., Mantovani, A. “Defective expression of the monocyte chemotactic protein 1 receptor CCF2 in macrophages associated with human ovarian carcinoma”. J. Immunology. 2000; 164: 733-8.

15. Kypson, A., Hendrickson, S., Akhter, S., Wilson, K., McDonald, P., Lilly, R., Dolber, P., Glower, D., Lefkowitz, R., Koch, W. “Adenovirus-mediated gene transfer of the B2 AR to donor hearts enhances cardiac function”. Gene Therapy. 1999; 6: 1298-304.

16. Dom, G. W., Tepe, N. M., Lorenz, J. N., Kock, W. J., Ligget, S. B. “Low and high level transgenic expression of B2AR differentially affect cardiac hypertrophy and function in Galpha q-overexpressing mice”. PNAS. 1999; 96: 6400-5.

17. J. Wess. “G protein coupled receptor: molecular mechanisms involved in receptor activation and selectivity of G-protein recognition”. FASEB. 1997; 11:346-354.

18. Whitney, M, Rockenstein, E, Cantin, G., Knapp, T., Zlokarnik, G., Sanders, P., Durick, K., Craig, F.F., and Negulescu, P. A. “A genome-wide functional assay of signal transduction in living mammalian cells”. Nature Biotech. 1998; 16: 1329-1333.

19. BD Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W.J., Kenakin, T. “Constitutive Receptor systems for drug discovery”. J. Pharmacol. Toxicol. Methods 1999; 42: 199-206.

21. Blahos, J., Fischer,T., Brabet, I., Stauffer,D., Rovelli, G., Bockaert, J., and Pin, J.-P. “A novel Site on the G alpha-protein that Rocognized Heptahelical Receptors”. J. Biol. Chem. 2001; 275, No. 5, 3262-69.

22. Offermanns, S. & Simon, M. I. “G alpha 15 and G alpha 16 Couple a Wide Variety of Receptors to Phospholipase C”. J. Biol. Chem. 1995; 270, No. 25, 15175-80.

23. Bockaert, J., Claeysen, S., Becamel, C., Pinloche, S., and Dumuis, A. (2002). G protein-coupled receptors: dominant players in cell-cell communication. Int Rev Cytol 212, 63-132.

24. Civelli, O. (1998). Functional genomics, the search for novel neurotransmitters and neuropeptides. FEBS Letters 430, 55-58.

25. Hinuma, S., Onda, H., and Fujino, M. (1999). The quest for novel bioactive peptides utilizing orphan seven-transmembrane-domain receptors. J Mol Med 77, 495-504.

26. Howard, A. D., McAllister, G., Feighner, S. D., Liu, Q., Nargund, R. P., Van der Ploeg, L. H., and Patchett, A. A. (2001). Orphan G-protein-coupled receptors and natural ligand discovery. Trends Pharmacol Sci 22, 132-140.

27. Lander, E. S., Linton, L. M., Birren, B., Nusbaum, C., Zody, M. C., Baldwin, J., Devon, K., Dewar, K., Doyle, M., FitzHugh, W., et al. (2001). Initial sequencing and analysis of the human genome. Nature 409, 860-921.

28. Milligan, G., Marshall, F., and Rees, S. (1996). G(16) as a universal G protein adapter: implications for agonist screening strategies. Trends in Pharmacological Sciences 17, 235-237.

29. Milligan, G., and Rees, S. (1999). Chimaeric G alpha proteins: their potential use in drug discovery. Trends Pharmacol Sci 20, 118-124.

30. Offermanns, S., and Simon, M. I. (1995). G alpha 15 and G alpha 16 couple a wide variety of receptors to phospholipase C. J Biol Chem 270, 15175-15180.

31. Pierce, K. L., Premont, R. T., and Lefkowitz, R. J. (2002). Seven- transmembrane receptors. Nature Reviews Molecular Cell Biology 3, 639-650.

32. Sambrook, J., and Russell, D. W. (2001). Molecular Cloning: A Laboratory Manual, 3rd edn (Cold Spring Harbor, Cold Spring Harbor Laboratory Press).

33. Szekeres, P. G. (2002). Functional assays for identifying ligands at orphan G protein-coupled receptors. Receptors and Channels 8, 297-308.

34. Venter, J. C., Adams, M. D., Myers, E. W., Li, P. W., Mural, R. J., Sutton, G. G., Smith, H. O., Yandell, M., Evans, C. A., Holt, R. A., et al. (2001). The sequence of the human genome. Science 291, 1304-1351.

35. Nestler E J, Barrot M, DiLeone R J, Eisch A J, Gold S J, Monteggia L M. Neurobiology of depression. Neuron. 2002 Mar. 28;34(1): 13-25.

36. Charney, D. S., Nestler, E. J., Bunney, B. S. Neurobiology of Mental Illness. Oxford University Press. Copyright 1999, New York, N.Y.

37. Martin, J. H. Neuroanatomy: Text and Atlas. Elsevier Science Publishing Co.

38. Barrot M, Olivier J D, Perrotti L I, DiLeone R J, Berton O, Eisch A J, Impey S, Storm D R, Neve R L, Yin J C, Zachariou V, Nestler E J. CREB activity in the nucleus accumbens shell controls gating of behavioral responses to emotional stimuli. Proc Natl Acad Sci USA. 2002 Aug. 20;99(17): 11435-40.

39. Phillips A G, Ahn S, Howland J G. Amygdalar control of the mesocorticolimbic dopamine system: parallel pathways to motivated behavior. Neurosci Biobehav Rev. 2003 October;27(6):543-54.

40. Robinson T. E. and Justice J. B. Jr, eds. (1991) Microdialysis in the Neurosciences, Vol. 7, Elsevier, Amsterdam.

41. Microdialysis—theory and application. Benveniste H, Huttemeier PC. Prog Neurobiol. 1990;35(3): 195-215.

42. Microdialysis monitoring of catecholamines and excitatory amino acids in the rat and mouse brain: recent developments based on capillary electrophoresis with laser-induced fluorescence detection-a mini-review. Parrot S, Bert L, Mouly-Badina L, Sauvinet V, Colussi-Mas J, Lambas-Senas L, Robert F, Bouilloux J P, Suaud-Chagny M F, Denoroy L, Renaud B. Cell Mol Neurobiol. 2003 October;23(4-5):793-804.

43. Cryan J F, Markou A, Lucki I. Assessing antidepressant activity in rodents: recent developments and future needs. Trends Pharmacol Sci. 2002 May;23(5):238-45.

44. Dalvi A, Lucki I. Murine models of depression. Psychopharmacology (Berl). 1999 November; 147(1):14-6

45. Lucki I. The forced swimming test as a model for core and component behavioral effects of antidepressant drugs. Behav Pharmacol. 1997 November;8(6-7):523-32.

What is claimed is:

1. An isolated polynucleotide selected from the group consisting of:
(a) an isolated polynucleotide encoding a human G-protein coupled receptor, or functional fragment thereof, comprising the amino acid sequence as set forth in SEQ ID NO:2;
(b) An isolated composition comprising the polynucleotide according to
(a).
(c) An isolated polynucleotide comprising SEQ ID NO:1;
(d) An isolated polynucleotide having the nucleic acid sequence of ATCC Accession No. PTA-2966;
(e) An isolated polynucleotide having the nucleic acid sequence according to nucleotides 4 to 1524 of SEQ ID NO:1, wherein said nucleotides encode a polypeptide of SEQ ID NO:2 minus the start codon;
(f) An isolated polynucleotide having the nucleic acid sequence according to nucleotides 1 to 1524 of SEQ ID NO:1, wherein said nucleotides encode a polypeptide of SEQ ID NO:2 including the start codon;
(g) An isolated polynucleotide having the nucleic acid sequence according to nucleotides 1 to 1524 of SEQ ID NO:48, wherein said nucleotides encode a polypeptide of SEQ ID NO:49 including the start codon;
(h) An isolated polynucleotide having the nucleic acid sequence according to nucleotides 4 to 1524 of SEQ ID NO:48, wherein said nucleotides encode a polypeptide of SEQ ID NO:49 minus the start codon;
(i) A polynucleotide which is fully complementary to the polynucleotide according to (a) thru (h); and
(j) A hybridization probe comprising the polynucleotide according to (a) thru (i).

2. An expression vector containing the polynucleotide according to claim 1.

3. A host cell containing the expression vector according to claim 2.

4. A substantially purified G-protein coupled receptor polypeptide selected from the group consisting of:
(a) A substantially purified G-protein coupled receptor polypeptide comprising an amino acid sequence as set forth in SEQ ID NO:2.
(b) The polypeptide according to (a), wherein the amino acid sequence differs from SEQ ID NO:2 only by conservative substitutions;
(c) An isolated and substantially purified G-protein coupled receptor polypeptide encoded by the nucleic acid sequence of ATCC Accession No. PTA-2966;
(d) An isolated polypeptide having the amino acid sequence according to amino acids 2 to 508 of SEQ ID NO:2, wherein said amino acid encode a polypeptide of SEQ ID NO:2 minus the start methionine;
(e) An isolated polypeptide having the amino acid sequence according to amino acids 1 to 508 of SEQ ID NO:2, wherein said amino acid encode a polypeptide of SEQ ID NO:2 including the start methionine;
(f) An isolated polypeptide having the amino acid sequence according to amino acids 2 to 508 of SEQ ID NO:49, wherein said amino acid encode a polypeptide of SEQ ID NO:2 minus the start methionine;
(g) An isolated polypeptide having the amino acid sequence according to amino acids 1 to 508 of SEQ ID NO:49, wherein said amino acid encode a polypeptide of SEQ ID NO:2 including the start methionine;
(h) A substantially purified fragment of the G-protein coupled receptor polypeptide according to any one of (a) to (g).

5. A substantially purified fusion protein comprising an amino acid sequence as set forth in SEQ ID NO:2 and an amino acid sequence of an Fc portion of a human immunoglobulin protein.

6. A pharmaceutical composition comprising the polypeptide, or a functional fragment thereof, according to claim 1, and a pharmaceutically acceptable diluent or excipient.

7. A purified antibody which binds specifically to the polypeptide according to claim 4, or an antigenic epitope thereof.

8. A method of screening for candidate compounds capable of modulating the activity of a G-protein coupled receptor polypeptide, comprising:
(a) contacting a test compound with a cell or tissue comprising an expression vector capable of expressing a polypeptide comprising an amino acid sequence as set forth in SEQ ID NO:2, or encoded by ATCC deposit PTA-2966, under conditions in which said polypeptide is expressed; and
(b) selecting as candidate modulating compounds those test compounds that modulate activity of the G-protein coupled receptor polypeptide.

9. The method according to claim 8 wherein said cells are CHO cells.

10. The method according to claim 9 wherein said cells comprise a vector comprising the coding sequence of the beta lactamase gene under the control of one or more NFAT response elements.

11. The method according to claim 11 wherein said cells further comprise a vector comprising the coding sequence of G alpha 15 under conditions wherein G alpha 15 is expressed.

12. The method according to claim 9 wherein said cells are HEK cells.

13. The method according to claim 12 wherein said cells express a polypeptide comprising an amino acid sequence set forth in SEQ ID NO:2, an amino acid sequence encoded by ATCC deposit PTA-2966, or beta lactamase, at either high or low levels of expression relative to the expression of a reference polypeptide.

14. The method according to claim 12 wherein said cells further comprise a vector comprising the coding sequence of the polypeptide provided as SEQ ID NO:103.

15. The method according to claim 12 wherein said candidate compound is selected from the group consisting of: a small molecule; a peptide; and an antisense molecule.

16. The method according to claim 15 wherein said candidate compound is an agonist.

17. The method according to claim 15 wherein said candidate compound is an antagonist.

18. A pharmaceutical composition comprising the candidate compound according to claim 15.

19. A method of treating a neurological disorder in a mammal comprising administration of the pharmaceutical composition of claim 18.

20. The method according to claim 19 wherein said disorder is a member of the group consisting of: disorders of the caudate nucleus selected from the group consisting of: a neurological disorder; nucleus accumbens disorders, caudate nucleus disorders, neurotransmitter disorders, neurotransmitter expression disorders, neurotransmitter release disorders, disorders associated with aberrant dopamine synthesis, disorders associated with aberrant dopamine release, disorders associated with aberrant dopamine function, disorders associated with aberrant opioid peptide synthesis, disorders associated with aberrant opioid peptide release, disorders associated with aberrant opioid peptide function, disorders associated with aberrant serotonin synthesis, disorders associated with aberrant serotonin release, disorders associated with aberrant serotonin function, disorders associated with aberrant GABA synthesis, disorders associated with aberrant GABA release, disorders associated with aberrant GABA function, disorders associated with the release of GABA from L-glutamic acid, schizophrenia, Parkinson's disease, progressive supranuclear palsy, Alzheimer's, affective disorders, depression, aggressive behavioral disorders, addictive disorders, sleep disorders, eating disorders neuropathic pain; psychotic disorders; fear, stress disorders, severe mental retardation; dyskinesias, Huntington's disease; Gilles dela Tourett's syndrome; Sydenham chorea; major depressive disorder; obsessive-compulsive disorder; movement type disorders; anxiety; schizophrenia; manic depression; delirium; dementia; brain cancer, proliferative disorder of the brain, and neoplastic diseases of the brain.

21. The method according to claim 20 wherein said disorder is an anxiolytic disorder.

22. The method according to claim 20 wherein said disorder is a disorder further associated with aberrant expression or activity of one or more of the following: p21, p27, and IkB.

23. An antisense compound 8 to 30 nucleotides in length that specifically hybridizes to a nucleic acid molecule encoding the human HGPRBMY8 polypeptide of the present invention, wherein said antisense compound inhibits the expression of the human HGPRBMY8 polypeptide.

24. The antisense compound of claim 23, wherein said antisense compound is a member of the group consisting of any one of the sequences provided as SEQ ID NO:115 to SEQ ID NO:124.

25. A peptide that specifically inhibits the activity of the human HGPRBMY8 polypeptide provided as SEQ ID NO:2, wherein said peptide is a member of the group consisting of any one of the sequences provided as SEQ ID NO:66 to SEQ ID NO:71.
